(19) United States
(12) Patent Application Publication
(10) Pub. No.: US 2025/0066290 A1
(43) Pub. Date: Feb. 27, 2025

(54) DHODH INHIBITORS CONTAINING A CARBOXYLIC ACID BIOISOSTERE

(71) Applicant: IMMUNIC AG, Gräfelfing (DE)

(72) Inventors: Christian GEGE, Ehingen (DE); Andreas MÜHLER, München (DE); Hella KOHLHOF, München (DE); Daniel VITT, Germering (DE)

(21) Appl. No.: 18/719,384

(22) PCT Filed: Dec. 23, 2022
(86) PCT No.: PCT/EP2022/087752

§ 371 Date: Jun. 13, 2024

(30) Foreign Application Priority Data
Dec. 23, 2021 (EP) 21217534.3
Oct. 12, 2022 (EP) 22201158.7

(51) Int. Cl. C07C 317/28; A61K 31/165; A61K 31/18; A61K 31/277; A61K 31/341; A61K 31/357; A61K 31/381; A61K 31/40; A61K 31/41; A61K 31/423; A61K 31/4245; A61K 31/426; A61K 31/437; A61K 31/4418; A61K 31/472; A61K 31/5375; A61K 45/06; C07C 233/60; C07C 255/16; C07C 255/46; C07C 317/26; C07D 207/34; C07D 213/64; C07D 217/02; C07D 257/04; C07D 263/56; C07D 265/30; C07D 271/07; C07D 277/68; C07D 307/30; C07D 317/60; C07D 333/38; C07D 405/04; C07D 471/04

(52) U.S. Cl. CPC C07C 317/28; A61K 31/165; A61K 31/18; A61K 31/277; A61K 31/341; A61K 31/357; A61K 31/381; A61K 31/40; A61K 31/41; A61K 31/423; A61K 31/4245; A61K 31/426; A61K 31/437; A61K 31/4418; A61K 31/472; A61K 31/5375; A61K 45/06; C07C 233/60; C07C 255/16; C07C 255/46; C07C 317/26; C07D 207/34; C07D 213/64; C07D 217/02; C07D 257/04; C07D 263/56; C07D 265/30; C07D 271/07; C07D 277/68; C07D 307/30; C07D 317/60; C07D 333/38; C07D 405/04; C07D 471/04; C07C 2601/02; C07C 2601/10; C07C 2601/16; C07C 2602/44

(57) ABSTRACT

The invention relates to novel, optionally deuterated compounds of Formula (I)

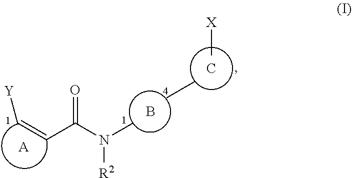

and their use as medicament.

### SUMMARY OF THE INVENTION

The present disclosure relates to novel dihydroorotate dehydrogenase (DHODH) inhibitors having a carboxylic acid bioisosteric moiety and being optionally deuterated, pharmaceutical formulations comprising them, a process for their preparation and their use as medicament, alone or in combination with one or more additional agents, for treating of various diseases, wherein the inhibition of DHODH is desirable.

### BACKGROUND OF THE INVENTION

Vidofludimus calcium (IMU-838) is a selective and potent second-generation dihydroorotate dehydrogenase (DHODH) oral immunomodulator being developed for the treatment of several chronic inflammatory diseases, including relapsing-remitting Multiple Sclerosis (rrMS):

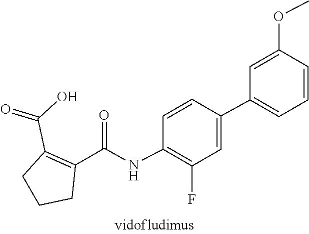 [SMILES: COc1cccc(-c2ccc(NC(=O)C3=C(C(=O)O)CCC3)c(F)c2)c1]

The mechanism of action of vidofludimus calcium, a small molecule selective immune modulator, is the inhibition of the intracellular metabolism of activated immune T- and B-cells by blocking the enzyme DHODH. The inhibition of the DHODH enzyme leads to metabolic stress in metabolically activated lymphocytes resulting in reduction in proinflammatory cytokines and subsequently to apoptosis of activated immune cells. Blocking of the DHODH enzyme activity has a selective effect to metabolically activated immune cells, to malignant cells and to virus-infected cells. Thus, DHODH inhibition should therefore not lead to general antiproliferative effects in other cells. IMU-838 as a second-generation DHODH inhibitor is being developed to separate the desired immunomodulatory effects from an undesirable side effect profile caused by off-target effects like neutropenia, alopecia and diarrhea. An additional benefit of DHODH inhibitors such as IMU-838 is their direct antiviral effect. During long-term treatment with immunosuppressive drugs, the reactivation of latent viruses has been observed. This can lead to serious infections, such as progressive multifocal leukoencephalopathy which can have a lethal outcome.

PP-001 is another DHODH inhibitor within the same structural class for the treatment of retinal diseases like uveitis, diabetic macular edema and retinal vein occlusion currently in clinical trials. In animal models the high effectiveness to treat dry eye disease and viral conjunctivitis has already been demonstrated.

So far, compounds from this structural class (e.g. IMU-838 or PP-001) contain a carboxylic acid functional group as an important constituent of the pharmacophore. However, the presence of this moiety can represent a liability. For instance, a diminished ability to passively diffuse across biological membranes can raise a significant challenge, particularly in the context of central nervous system drug discovery, where the blood-brain barrier can be relatively impermeable to negatively charged carboxylates. Furthermore, idiosyncratic drug toxicities arising from the metabolism of the carboxylic acid moiety (e.g. glucuronidation) have been linked to withdrawals of marketed drugs. The urate transporter 1 (URAT-1) is a urate transporter and urate-anion exchanger which regulates the level of urate in the blood. It is known, that drugs containing a carboxylic acids (e.g. probenecid, salicylic acid or fenofibric acid) are recognized by and interact with URAT-1 affecting urinary uric acid excretion. Also, at high vidofludimus doses a decrease in blood uric acid levels and an increase in urine red blood cell count were observed, in very rare cases, presenting as symptomatic hematuria during the first 7 days of treatment (WO2019/101888). This effect is caused due to interaction of vidofludimus with URAT-1 (Drugs R&D 2019; 19:351).

Therefore, there is still a need to develop novel DHODH inhibitors. In particular, there is a need to develop DHODH inhibitors with improved pharmacokinetic and pharmacodynamic properties. This can be accomplished by replacing hydrogen atom(s) by deuterium atom(s) in the molecule. The covalent C—H bond is weaker than an otherwise identical C-D bond due to the kinetic isotope effect. The breaking of C—H bonds is a common feature of drug metabolism and breaking of an analogous C-D bond can be more difficult and so decreases the rate of metabolism. Replacement of H with D in small molecules can lead to significant reduction in metabolism leading to beneficial changes in the biological effect of drugs. Replacement may also have the effect of lowering toxicity by reducing the formation of a toxic metabolite (J. Med. Chem. 2019; 62:5276). Deuterated analogs share the beneficial mechanism of action, however are expected to be metabolized slower and with less variability between patients compared with the non-deuterated matched pair. It is generally believed that a differentiated pharmacokinetic profile could enable potentially improved efficacy, less frequent dosing, improved tolerability, reduced interpatient variability in drug metabolism and reduced drug-drug interactions.

### PRIOR ART

Compounds of Formula (I) containing a carboxylic acid instead of residue Y are described in WO2004/056746, WO2004/056747, WO2004/056797, WO2010/052027, WO2010/128050, WO2012/001148, WO2012/001151, WO2015/169944, WO2015/154820, WO2018/177151, WO2019/170848, WO2019/101888, WO2019/175396 as well as in Bioorg. Med. Chem. Lett. 2004; 14:55, Bioorg. Med. Chem. Lett. 2005; 15:4854, Bioorg. Med. Chem. Lett. 2006; 16:267 and J. Med. Chem. 2006; 49:1239. Deuterated compounds of Formula (I) containing a carboxylic acid instead of residue Y have not yet been described. Also, compounds of Formula (I) containing an acidic bioisosteric functionality has not yet been described except for hydroxamic acid Example 4 from WO2004/056746:

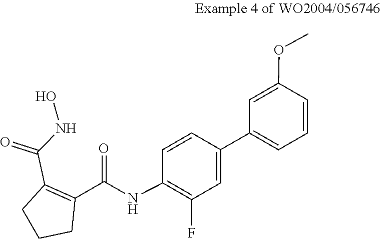 [SMILES: COc1cccc(-c2ccc(NC(=O)C3=C(C(=O)NO)CCC3)c(F)c2)c1]

The human DHODH inhibitory activity of this Example 4 is ranked within the worst category of IC₅₀>5 μM in the patent application, whereas the matched pair containing a carboxylic acid (vidofludimus) is described to have an IC₅₀<0.8 μM (WO2003/006425) and more precisely to have an IC₅₀ of 0.134 μM (Bioorg. Med. Chem. Lett. 2005; 15:4854). As outlined in the experimental section, we surprisingly found that by replacement of the carboxylic acid moiety by other acidic bioisoteric moieties, DHODH inhibitors with beneficial properties (e.g. improved DHODH inhibitory activity, reduced lipophilicity, improved microsomal stability/clearance and/or bioavailability) can be obtained.

### BRIEF DESCRIPTION OF THE DRAWING

FIG. 1 depicts a representative result of an experiment wherein Example 1 is combined with the nucleoside analogue EIDD-1931 (CAS: 3258-02-4). The data shows a synergistic antiviral effect on SARS-CoV-2 at different doses.

FIG. 2 depicts representative human DHODH inhibition curves for Example 4/33 and matched pairs mentioned in the prior art.

### SUMMARY OF THE INVENTION

The present invention relates to compounds according to Formula (I)

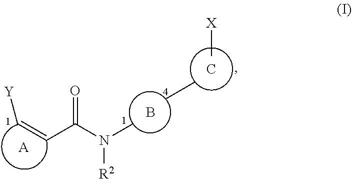

or an enantiomer, diastereomer, tautomer, solvate, or pharmaceutically acceptable salt thereof, wherein cycle A, B, C and residues X, Y and R² are defined as in claim 1, with the proviso, that the following structure is excluded:

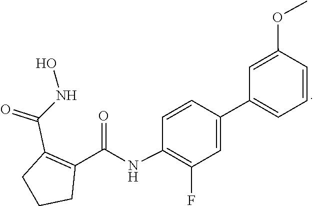 [SMILES: COc1cccc(-c2ccc(NC(=O)C3=C(C(=O)NO)CCC3)c(F)c2)c1]

The compounds of the present invention have a similar or better DHODH inhibitory activity compared to the known DHODH inhibitors. Furthermore, the compounds of the present invention exhibit additional beneficial properties like reduced lipophilicity, reduced interaction with the URAT1 transporter, improved microsomal stability/clearance and/or improved bioavailability due to the carboxylic acid bioisosteric moiety. Additional improved microsomal stability and/or improved bioavailability can be obtained when used as medicament due to the replacement of hydrogen to deuterium at certain positions.

Thus, the present invention further relates to a pharmaceutical composition comprising a compound according to Formula (I) and at least one pharmaceutically acceptable carrier or excipient.

The present invention is further directed to compounds according to Formula (I) for use in the prophylaxis and/or treatment of diseases mediated by DHODH.

Accordingly, the present invention relates to the prophylaxis and/or treatment of the disease, disorder, therapeutic indication or medical condition which is selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy. More specifically, the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

The present invention is further directed to a pharmaceutical composition comprising a compound according to Formula (I) and one or more additional therapeutic agents selected from anti-inflammatory agents, anti-viral agents, immunosuppressive and/or immunomodulatory agents, steroids, non-steroidal anti-inflammatory agents, antihistamines, analgesics and suitable mixtures thereof.

### DETAILED DESCRIPTION OF THE INVENTION

Compound 2-((3-fluoro-3′-methoxy-[1,1′-biphenyl]-4-yl)carbamoyl)cyclopent-1-ene-1-carboxylic acid, also known as vidofludimus is an orally administered DHODH inhibitor. The calcium salt of vidofludimus is known as IMU-838. IMU-838 is currently in a Phase 3 clinical trial for the treatment of MS and also in clinical trials for ulcerative colitis and primary sclerosing cholangitis.

Compound 3-((2,3,5,6-tetrafluoro-3′-(trifluoromethoxy)-[1,1′-biphenyl]-4-yl)carbamoyl)thiophene-2-carboxylic acid, also known as PP-001 is a topically administered DHODH inhibitor. PP-001 is currently in clinical trials for the treatment of keratoconjunctivitis and non-infectious uveitis.

Vidofludimus, IMU-838 and PP-001 has generally been well-tolerated in several clinical trials. Despite the potential beneficial activities of vidofludimus, IMU-838 and PP-001, there is a continuing need for new compounds to treat the aforementioned diseases and conditions that have improved off-target and drug metabolism and pharmacokinetic (DMPK) properties. Improved off-target and DMPK properties have the potential to result in positive changes in safety profile, efficacy and tolerability of compounds. Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

In one aspect the invention relates to a compound of Formula (I):

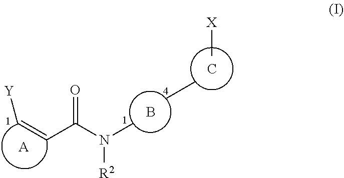

or an enantiomer, diastereomer, tautomer, solvate, or pharmaceutically acceptable salt thereof, wherein A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, —CN, —NO₂, oxo, —OH, C₁₋₄-alkyl, —O—C₁₋₄-alkyl, fluoro-C₁₋₄-alkyl and —O-fluoro-C₁₋₄-alkyl, ring A having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(═O)(═NR²³)ₘR²¹, CO₀₋₆-alkylene-NR²¹S(═O)ₓ(═NR²³)_{y}R²¹, C₀₋₆-alkylene-S(═O)ₓ(═NR²³)_{y}NR²¹R²², C₀₋₆-alkylene-NR²¹S(═O)ₓ(═NR²³)_{y}NR²¹R²², C₀₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹, C₀₋₆-alkylene-NR²¹R²²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl, and wherein the residue —NR² on ring B is in a 1,4-orientation with respect to ring C,
B having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-N³¹R³²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl; and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl, C having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X having one or more hydrogen atoms optionally replaced by deuterium;
Y is selected from —CONH—CN, —CONHOH, —CONHOR¹⁰, —CONR¹⁰OH, —C(═NOH)NR¹¹R¹², —CONHS(═O)ₓ(═NR¹³)_{y}R¹⁰, —CONHS(═O)_{y}(═NR¹³)_{y}NR¹¹R¹², —SO₃H, —S(═O)ₓ(═NR¹³)_{y}NHCOR¹⁰, —S(═O)ₓ(═NR¹³)_{y}NHR¹¹, —P(═O)(OH)₂, —P(═O)(NR¹¹R¹²)OH, —P(═O)R¹¹(OH), —B(OH)₂,

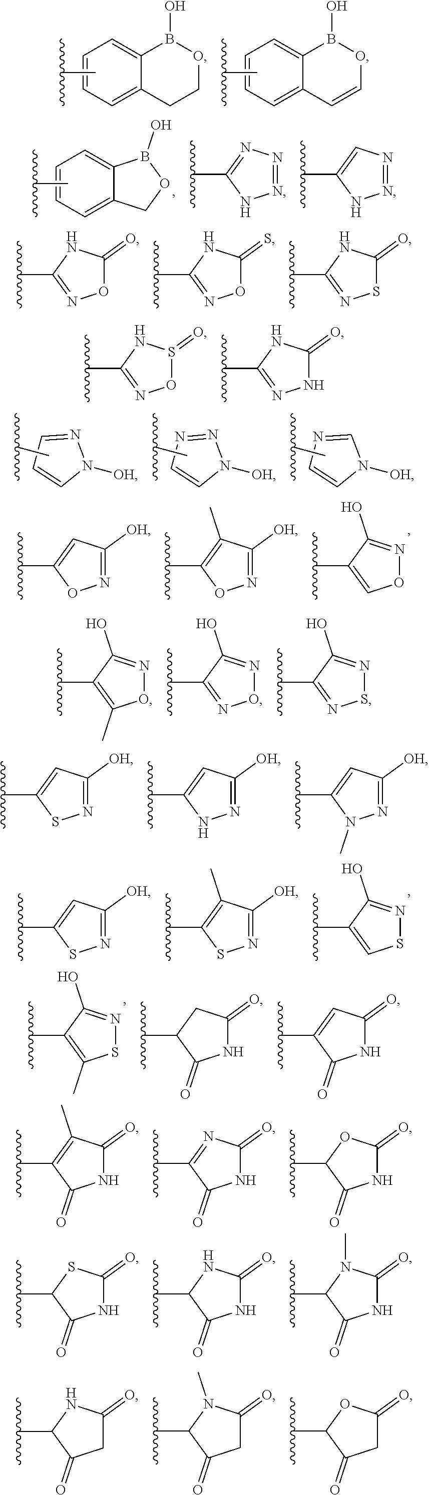

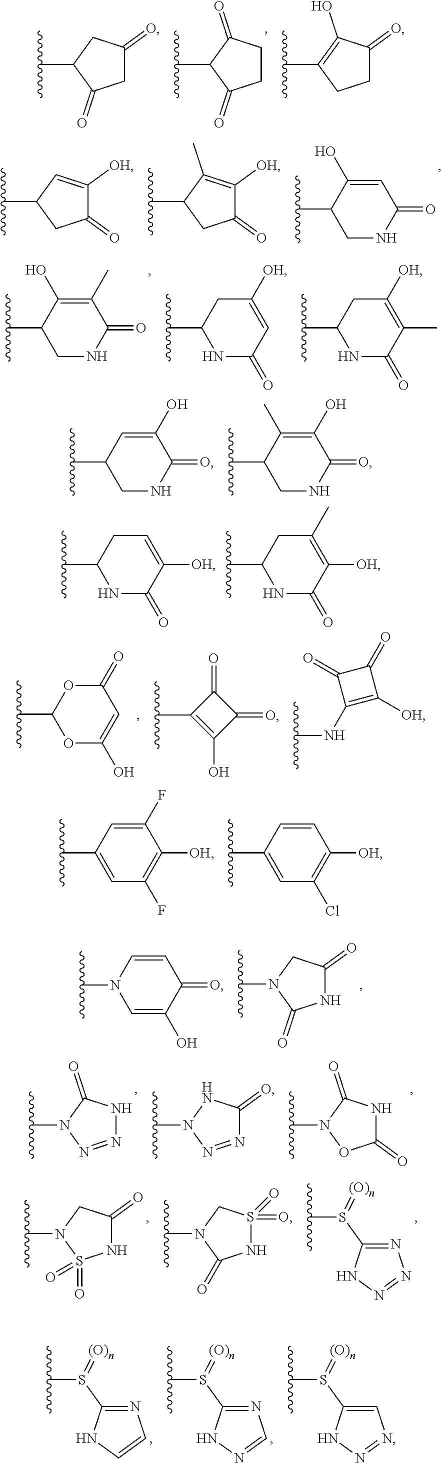

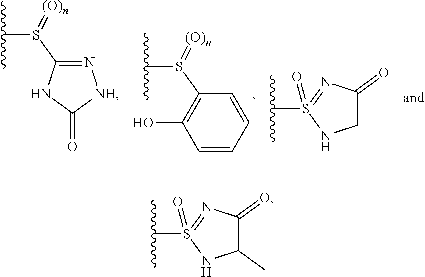

Y having one or more hydrogen atoms optionally replaced by deuterium;
R^{z} is selected from H and C₁₋₆-alkyl,
wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² having one or more hydrogen atoms optionally replaced by deuterium;
R¹⁰ is selected from C₁₋₆-alkyl, 3- to 6-membered cycloalkyl or 3- to 6-membered heterocycloalkyl, wherein alkyl, cycloalkyl and heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹⁰ having one or more hydrogen atoms optionally replaced by deuterium;
R¹¹, R¹², R²¹, R²², R³¹, R³², R⁴¹, R⁴² are independently selected from H, C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹¹ and/or R¹² and/or R²¹ and/or R²² and/or R³¹ and/or R³² and/or R⁴¹ and/or R⁴² having one or more hydrogen atoms optionally replaced by deuterium;
or R¹¹ and R¹², R²¹ and R²², R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R¹¹ and/or R¹² and/or R²¹ and/or R²² and/or R¹ and/or R³² and/or R⁴¹ and/or R⁴² having one or more hydrogen atoms optionally replaced by deuterium;
R¹³, R²³, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹³ and/or R²³ and/or R³³ and/or R⁴³ having one or more hydrogen atoms optionally replaced by deuterium;
n, m, x, y are independently selected from 0 to 2;
with the proviso that the sum of integer m and n for the residue linked to the same sulfur atom is independently selected from 0 to 2;
with the proviso that the sum of integer x and y for the residue linked to the same sulfur atom is independently selected from 1 or 2;
and with the proviso, that the following structure is excluded:

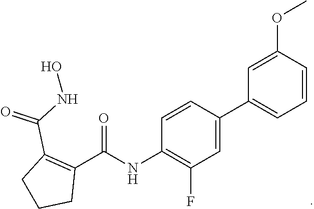 [SMILES: COc1cccc(-c2ccc(NC(=O)C3=C(C(=O)NO)CCC3)c(F)c2)c1]

In a more particular embodiment the compound is represented by Formula (I) or a solvate or pharmaceutically acceptable salt thereof, wherein

Y is selected from —CONH—CN, —CONHOR¹⁰, —CONR¹⁰OH, —C(═NOH)NR¹¹R¹², —CONHS(═O)ₓ(═NR¹³)_{y}R¹⁰, —CONHS(═O)_{y}(═NR¹³)_{y}NR¹¹R¹²,

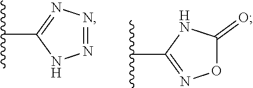

R¹⁰ is selected from C₁₋₃-alkyl, cyclopropyl or oxetan-3-yl,
wherein alkyl, cyclopropyl or oxetan-3-yl is unsubstituted or substituted with 1 to 3 substituents independently selected from F, —CN, Me, CHF₂, CF₃, —OH, oxo, —OMe, —OCHF₂ and —OCF₃, R¹⁰ having one or more hydrogen atoms optionally replaced by deuterium;
R¹¹ and R¹² are independently selected from H or C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹¹ and/or R¹² having one or more hydrogen atoms optionally replaced by deuterium;
R¹³ is selected from H, —CN and C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹³ having one or more hydrogen atoms optionally replaced by deuterium;
x is 1 and y is 1 or x is 2 and y is 0.

In a particular embodiment, the compound is represented by Formula (I) or a solvate or pharmaceutically acceptable salt thereof, wherein

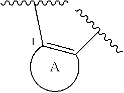

is selected from

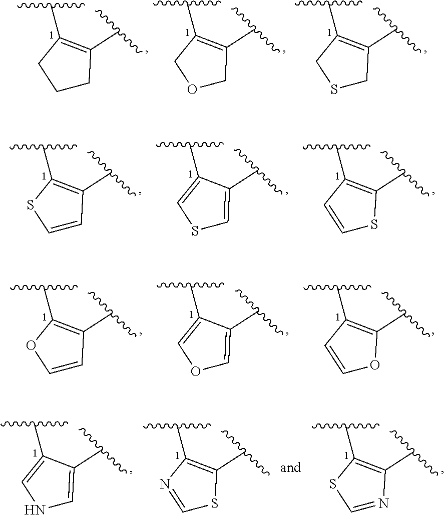

wherein ring A is unsubstituted or substituted with 1 to 2 substituents independently selected from the group consisting of halogen, oxo, —OH, C₁₋₄-alkyl, —O—C₁₋₄-alkyl, fluoro-C₁₋₄-alkyl and —O-fluoro-C₁₋₄-alkyl, ring A having one or more hydrogen atoms in alkyl optionally replaced by deuterium; and R² is H.

In a particular embodiment, the compound is represented by Formula (I) or a solvate or pharmaceutically acceptable salt thereof, wherein

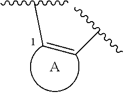

is selected from

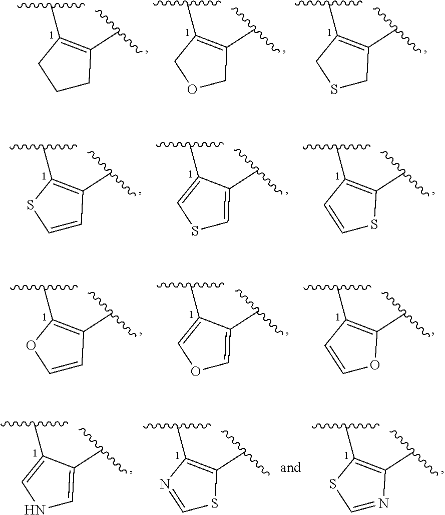

wherein ring A is unsubstituted or substituted with 1 to 2 substituents independently selected from the group consisting of fluoro and methyl; and R² is H.

In a particular embodiment, the compound is represented by Formula (I) or a solvate or pharmaceutically acceptable salt thereof, wherein

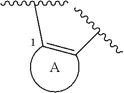

is selected from

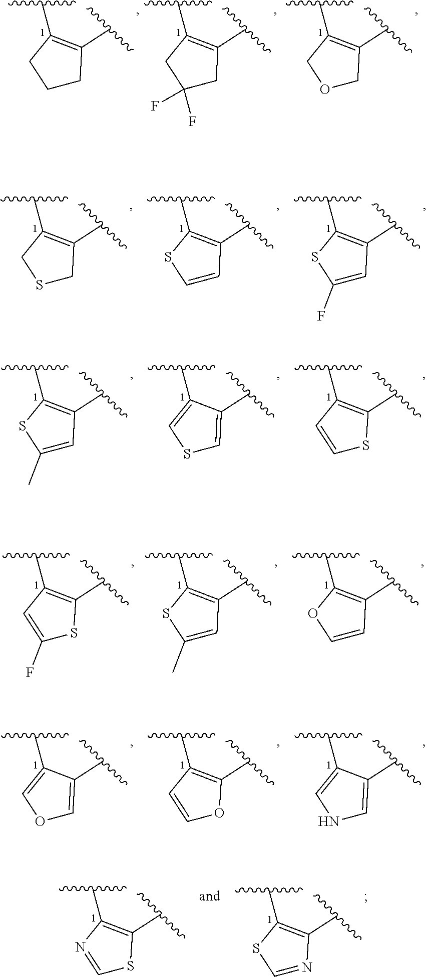

### and R² is H.

In a particular embodiment, the compound is represented by Formula (I) or a solvate or pharmaceutically acceptable salt thereof, wherein

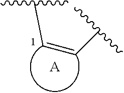

is selected from

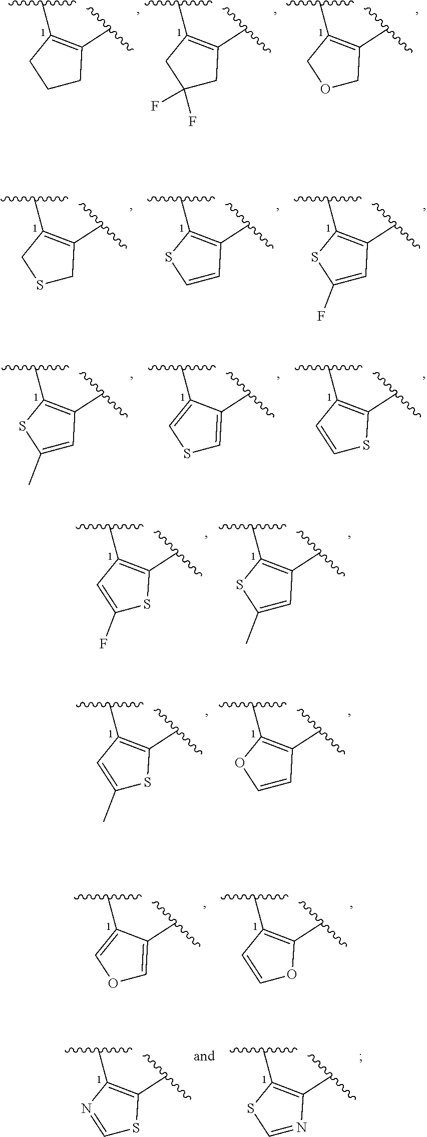

### and R² is H.

In a particular embodiment, the compound is represented by Formula (I) or a solvate or pharmaceutically acceptable salt thereof, wherein

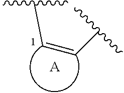

is selected from

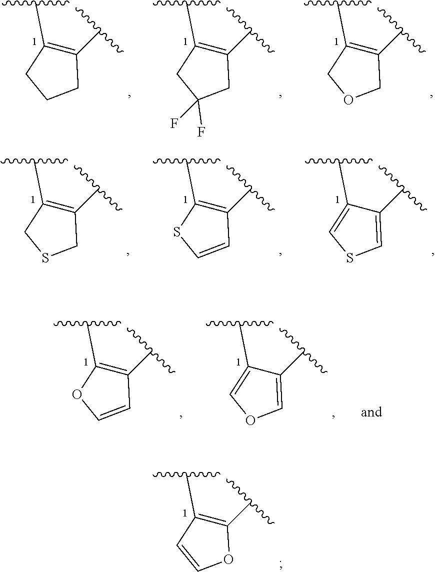

### and R² is H.

In a more particular embodiment, the compound is represented by Formula (I) or a solvate or pharmaceutically acceptable salt thereof, wherein

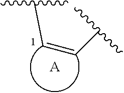

is selected from

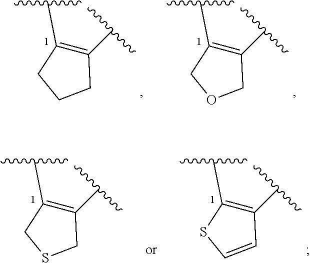 [SMILES: *C1=C(*)CCC1 ; *C1=C(*)COC1 ; *C1=C(*)CSC1 ; *c1ccsc1*]

### and R² is H.

In a particular embodiment, the compound is represented by Formula (I) or a solvate or pharmaceutically acceptable salt thereof, wherein

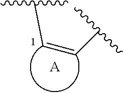

is selected from

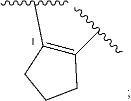 [SMILES: *C1=C(*)CCC1]

and R² is H. In an equally particular embodiment, the compound is represented by Formula (I) or a solvate or pharmaceutically acceptable salt thereof, wherein

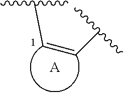

is selected from

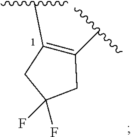 [SMILES: *C1=C(*)CC(F)(F)C1]

### and R² is H.

In an equally particular embodiment, the compound is represented by Formula (I) or a solvate or pharmaceutically acceptable salt thereof, wherein

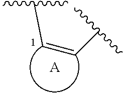

is selected from

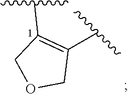 [SMILES: *C1=C(*)COC1]

### and R² is H.

In an equally particular embodiment, the compound is represented by Formula (I) or a solvate or pharmaceutically acceptable salt thereof, wherein

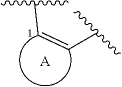

is selected from

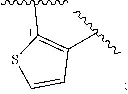 [SMILES: *c1ccsc1*]

### and R² is H.

In an equally particular embodiment, the compound is represented by Formula (I) or a solvate or pharmaceutically acceptable salt thereof, wherein

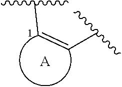

is selected from

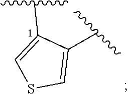 [SMILES: *c1cscc1*]

### and R² is H.

In a more particular embodiment, the compound is represented by Formula (I) or a solvate or pharmaceutically acceptable salt thereof, wherein one or more hydrogen atom(s) in any substituent is replaced by deuterium.

In a more particular embodiment, the compound is represented by Formula (I) or a solvate or pharmaceutically acceptable salt thereof, wherein one or more hydrogen atom(s) in any substituent is replaced by deuterium, provided, that the level of deuterium incorporation at each substituent designated as deuterium is at least 52.5%.

In a more particular embodiment, the compound is represented by Formula (I) or a solvate or pharmaceutically acceptable salt thereof, wherein one or more hydrogen atom(s) in ring C or any substituent of ring C is replaced by deuterium.

In a most particular embodiment, the compound is represented by Formula (I) or a solvate or pharmaceutically acceptable salt thereof, wherein one or more hydrogen atom(s) in residue X is replaced by deuterium.

In an upmost particular embodiment, the compound is represented by Formula (I) or a solvate or pharmaceutically acceptable salt thereof, wherein residue X is OCD₃.

In one particular embodiment, the compound is represented by Formula (I) or a solvate or pharmaceutically acceptable salt thereof, wherein one or more hydrogen atom(s) in residue Y is replaced by deuterium.

In a particular embodiment, the compound is represented by Formula (I) or a solvate or pharmaceutically acceptable salt thereof, wherein Y is selected from —CONH—CN, —CONHOH, —CONHOR¹⁰, —CONR¹⁰OH, —C(═NOH)NR¹¹R¹², —CONHS(═O)ₓ(═NR³)_{y}R¹⁰, —CONHS(═O)_{y}(═NR¹³)_{y}NR¹¹R¹², —SO₃H, —S(═O)ₓ(═NR¹³)_{y}NHCOR¹⁰, —S(═O)ₓ(═NR¹³)_{y}NHR¹¹, —P(═O)(OH)₂, —P(═O)(NR¹¹R¹²)OH, —P(═O)R¹¹(OH), —B(OH)₂,

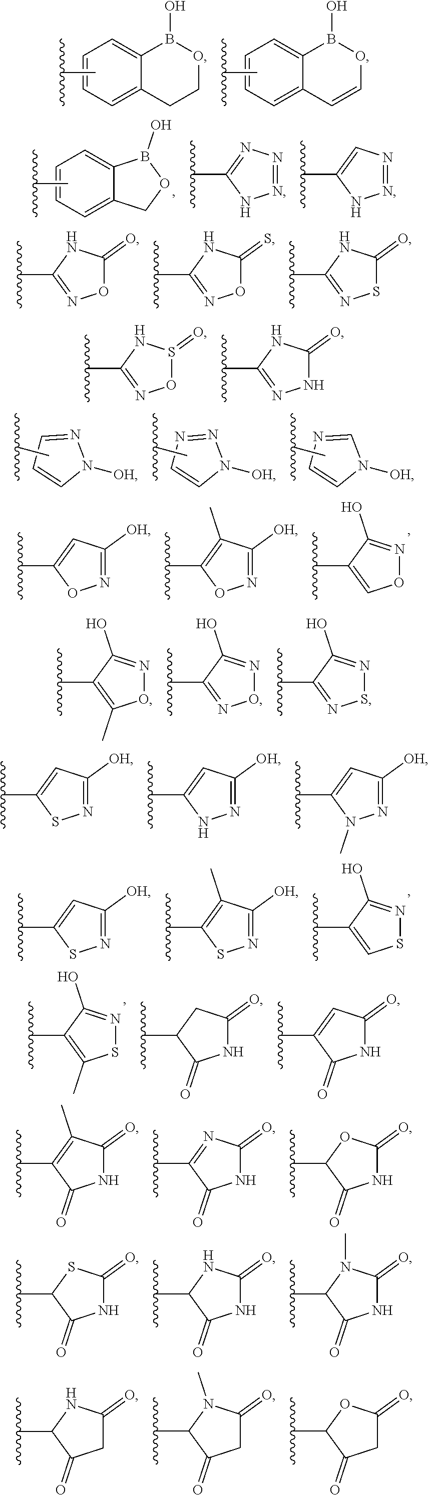

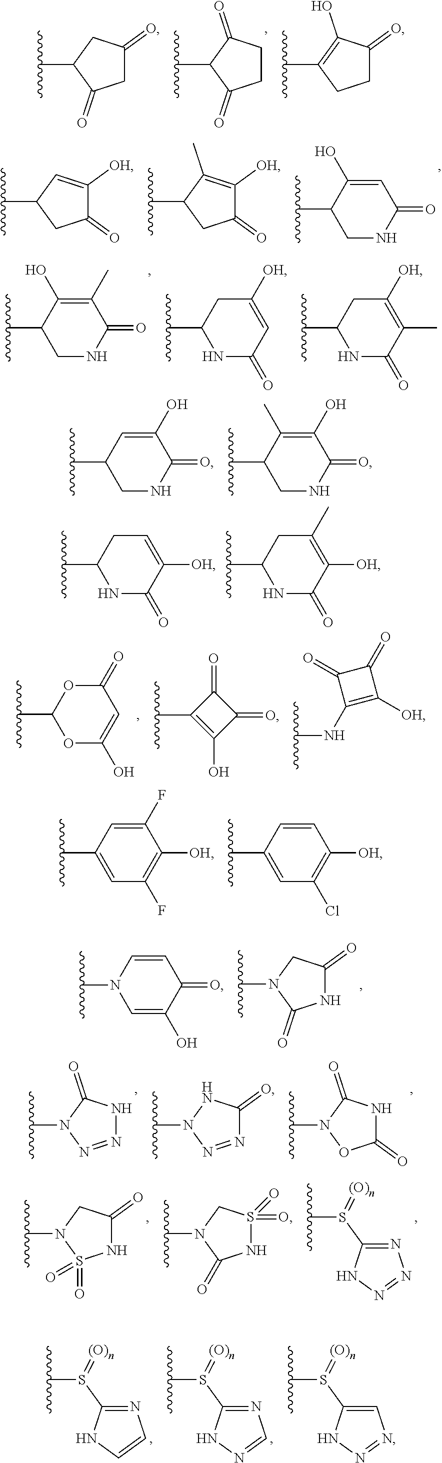

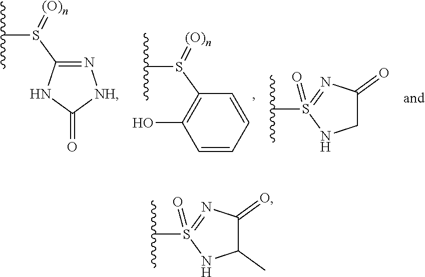

with Y having one or more hydrogen atoms optionally replaced by deuterium.

In a more particular embodiment, the compound is represented by Formula (I) or a solvate or pharmaceutically acceptable salt thereof, wherein Y is selected from —CONH—CN, —CONHOR¹⁰, —C(═NOH)NR¹¹R¹², —CONHS(═O)ₓ(═NR¹³)_{y}R¹⁰, —CONHS(═O)_{y}(═NR¹³)_{y}NR¹¹R¹², —SO₃H, —S(═O)ₓ(═NR¹³)_{y}NHCOR¹⁰, —S(═O)ₓ(═NR¹³)_{y}NHR¹¹, —P(═O)(OH)₂, —P(═O)(NR¹¹R¹²)OH, —P(═O)R¹¹(OH), —B(OH)₂,

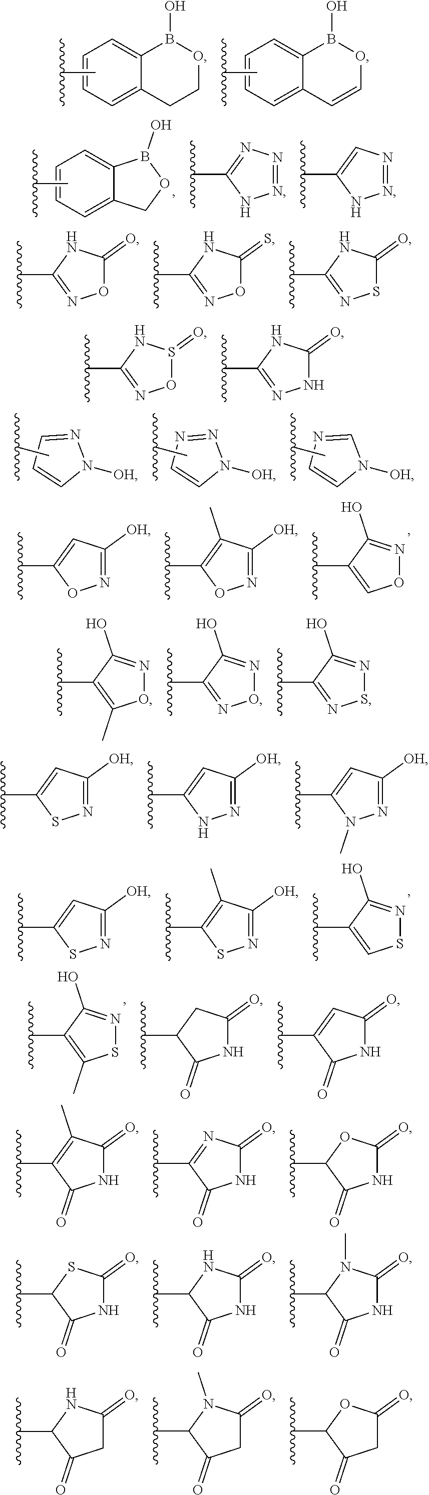

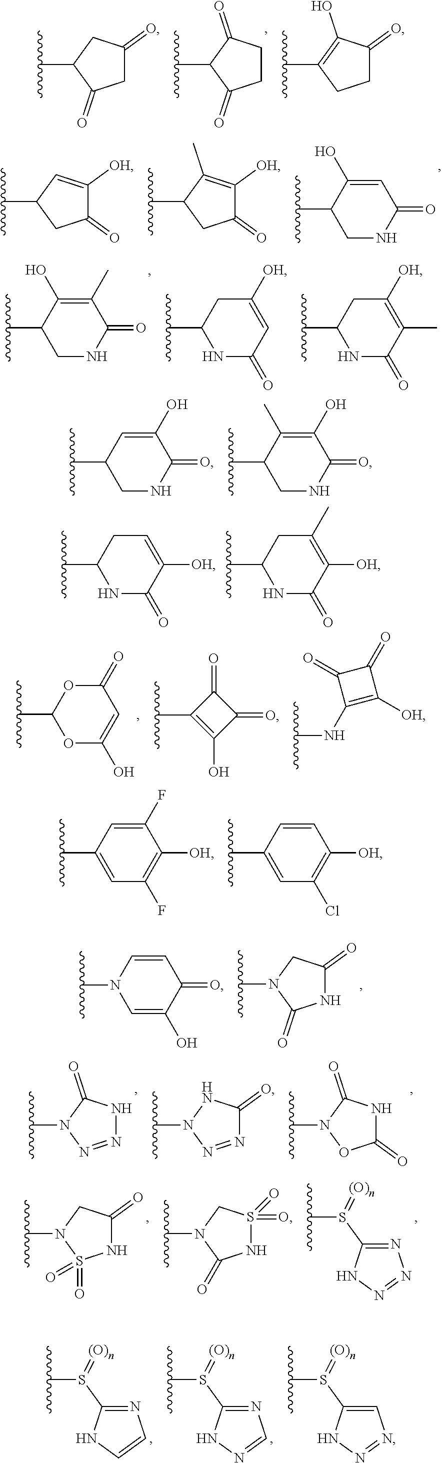

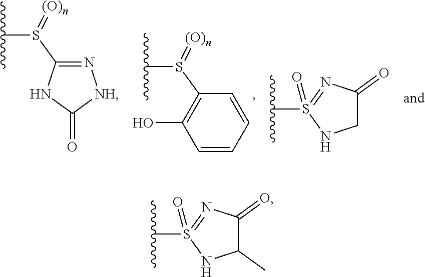

with Y having one or more hydrogen atoms optionally replaced by deuterium.

In one embodiment, the compound is represented by Formula (I) or a solvate or pharmaceutically acceptable salt thereof, wherein Y is selected from CONH—CN, —CONHOR¹⁰, —C(═NOH)NR¹¹R¹², —CONHS(═O)ₓ(═NR¹³)_{y}R¹⁰, —CONHS(═O)_{y}(═NR¹³)_{y}NR¹¹R¹²,

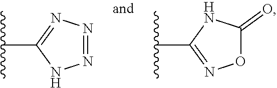 [SMILES: *c1nnn[nH]1 ; *c1noc(=O)[nH]1]

with Y having one or more hydrogen atoms optionally replaced by deuterium.

In an even more particular embodiment, the compound is represented by Formula (I) or a solvate or pharmaceutically acceptable salt thereof, wherein Y is selected from —CONH—CN, —CONHOR¹⁰, —CONHS(═O)ₓ(═NR¹³)_{y}R¹⁰, —CONHS(═O)_{y}(═NR¹³)_{y}NR¹¹R¹²,

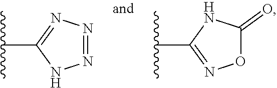 [SMILES: *c1nnn[nH]1 ; *c1noc(=O)[nH]1]

with Y having one or more hydrogen atoms optionally replaced by deuterium.

In an even most particular embodiment, the compound is represented by Formula (I) or a solvate or pharmaceutically acceptable salt thereof, wherein Y is selected from

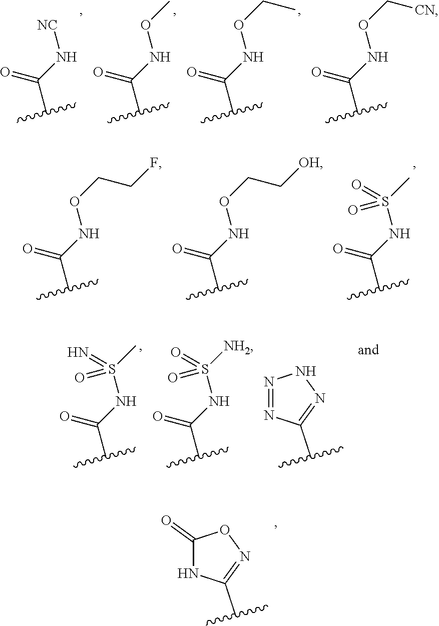

with Y having one or more hydrogen atoms in the alkyl moiety optionally replaced by deuterium.

In an similar even most particular embodiment, the compound is represented by Formula (I) or a solvate or pharmaceutically acceptable salt thereof, wherein Y is selected from

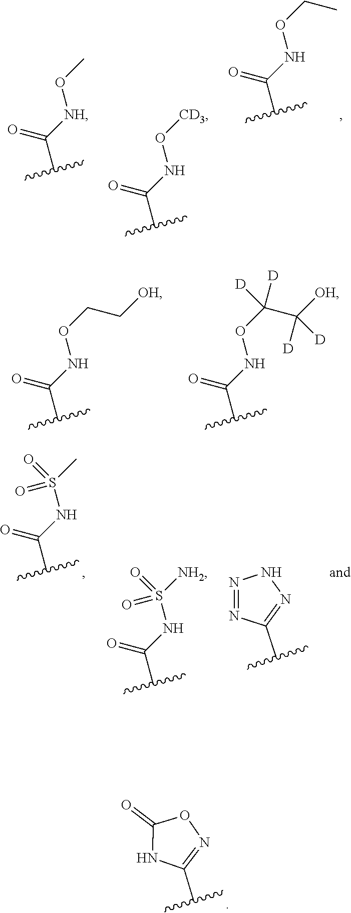

In one embodiment, Y is —CONHOR¹⁰, with R¹⁰ is selected from C₁₋₆-alkyl, 3- to 6-membered cycloalkyl or 3- to 6-membered heterocycloalkyl, wherein alkyl, cycloalkyl and heterocycloalkyl is unsubstituted or substituted with 1 to 3 substituents independently selected from halogen, —CN, C₁₋₄-alkyl, halo-C₁₋₄ alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S; and R¹⁰ having one or more hydrogen atoms optionally replaced by deuterium.

In one particular embodiment, Y is —CONHOR¹⁰, with R¹⁰ is selected from C₁₋₆-alkyl, which is optionally substituted with 1 to 3 substituents independently selected from fluoro, —CN, —OH, oxo, —O—C₁₋₄-alkyl and —O-halo-C₁₋₄-alkyl; and R¹⁰ having one or more hydrogen atoms optionally replaced by deuterium.

In a more particular embodiment, Y is —CONHOR¹⁰, with R¹⁰ is selected from C₁₋₃-alkyl, which is optionally substituted with 1 to 3 substituents independently selected from fluoro and —OH; and R¹⁰ having one or more hydrogen atoms optionally replaced by deuterium.

In one embodiment, Y is —CONHS(═O)₂R¹⁰, with R¹⁰ is selected from C₁₋₃-alkyl, which is optionally substituted with 1 to 3 fluoro substituents; and R¹⁰ having one or more hydrogen atoms optionally replaced by deuterium.

In a particular embodiment, Y is —CONHS(═O)₂CH₃ or CONHS(═O)₂CD₃.

In a more particular embodiment, Y is —CONHS(═O)₂CH₃.

In another particular embodiment, Y is —CONHS(═O)₂NH₂.

In one embodiment, R¹⁰ is selected from C₁₋₃-alkyl, cyclopropyl or oxetan-3-yl, wherein alkyl, cyclopropyl or oxetan-3-yl is unsubstituted or substituted with 1 to 3 substituents independently selected from F, —CN, Me, CHF₂, CF₃, —OH, oxo, —OMe, —OCHF₂ and —OCF₃; R¹⁰ having one or more hydrogen atoms optionally replaced by deuterium.

In one embodiment, R¹⁰ is selected from C₁₋₆-alkyl, 3- to 6-membered cycloalkyl or 3- to 6-membered heterocycloalkyl, wherein alkyl, cycloalkyl and heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S; R¹⁰ having one or more hydrogen atoms optionally replaced by deuterium.

In a particular embodiment, R¹⁰ is C₁₋₆-alkyl, which is unsubstituted or substituted with 1 to 3 substituents independently selected from halogen, —CN, C₁₋₄-alkyl, halo-C₁₋₄-alkyl, —OH, oxo, —O—C₁₋₄-alkyl and —O-halo-C₁₋₄-alkyl; R¹⁰ having one or more hydrogen atoms optionally replaced by deuterium.

In a more particular embodiment, R¹⁰ is C₁₋₃-alkyl, which is unsubstituted or substituted with 1 to 3 substituents independently selected from fluoro, —CN and —OH; R¹⁰ having one or more hydrogen atoms optionally replaced by deuterium.

In an even more particular embodiment, R¹⁰ is CH₃, CD₃, CH₂CH₂OH or CD₂CD₂OH.

In one embodiment, R¹¹ and R¹² are independently selected from H or C₁₋₃-alkyl, wherein alkyl is unsubstituted or substituted with 1 to 3 substituents independently selected from F, —CN, Me, CHF₂, CF₃, —OH, oxo, —OMe, —OCHF₂ and —OCF₃; R¹¹ and/or R¹² having one or more hydrogen atoms optionally replaced by deuterium.

In a particular embodiment, R¹¹ and R¹² are independently selected from H, CH₃, CD₃.

In a more particular embodiment, R¹¹ and R¹² are H.

In one embodiment, R¹³ is selected from H, —CN and C₁₋₃-alkyl, wherein alkyl is unsubstituted or substituted with 1 to 3 substituents independently selected from F, —CN, Me, CHF₂, CF₃, —OH, oxo, —OMe, —OCHF₂ and —OCF₃; R¹³ having one or more hydrogen atoms optionally replaced by deuterium.

In a more particular embodiment, R¹³ is H.

In one embodiment, R²¹, R²², R³¹, R³², R⁴¹, R⁴² are independently selected from H, CH₃, CD₃.

In a more particular embodiment, the compound is represented by Formula (I) or a solvate or pharmaceutically acceptable salt thereof, wherein B is phenyl, pyridyl, isoquinolinyl, quinolinyl, naphthyl, 2,3-dihydro-1H-indenyl, 1,2,3,4-tetrahydronaphthyl, bicyclo[2.2.2]octanyl or imidazo[1,2-a]pyridinyl, wherein phenyl, pyridyl, isoquinolinyl, quinolinyl, naphthyl, 2,3-dihydro-1H-indenyl, 1,2,3,4-tetrahydronaphthyl, bicyclo[2.2.2]octanyl or imidazo[1,2-a]pyridinyl is unsubstituted or substituted with 1 to 4 substituents independently selected from the group consisting of D, F, Cl, —CN, Me, CD₃, CHF₂ and CF₃; and wherein the residue —NR² on ring B is in a 1,4-orientation with respect to ring C.

In a more particular embodiment, the compound is represented by Formula (I) or a solvate or pharmaceutically acceptable salt thereof, wherein B is phenyl or pyridyl, wherein phenyl or pyridyl is unsubstituted or substituted with 1 to 4 substituents independently selected from the group consisting of D, F, Cl, —CN, Me, CD₃, CHF₂ and CF₃; and wherein the residue —NR² on ring B is in a 1,4-orientation with respect to ring C.

In a more particular embodiment, the compound is represented by Formula (I) or a solvate or pharmaceutically acceptable salt thereof, wherein B is phenyl, wherein phenyl is unsubstituted or substituted with 1 to 4 substituents independently selected from the group consisting of D, F, Cl, —CN, Me, CD₃, OMe, OCD₃, CHF₂ and CF₃; and wherein the residue —NR² on ring B is in a 1,4-orientation with respect to ring C.

In a more particular embodiment, the compound is represented by Formula (I) or a solvate or pharmaceutically acceptable salt thereof, wherein B is phenyl, wherein phenyl is unsubstituted or substituted with 1 to 4 substituents independently selected from the group consisting of D, F, Cl, —CN, Me, CD₃, CHF₂ and CF₃; and wherein the residue —NR² on ring B is in a 1,4-orientation with respect to ring C.

In a more particular embodiment, the compound is represented by Formula (I) or a solvate or pharmaceutically acceptable salt thereof, wherein B is phenyl, wherein phenyl is unsubstituted or substituted with 1 to 4 fluoro substituents; and wherein the residue —NR² on ring B is in a 1,4-orientation with respect to ring C.

In a most particular embodiment, the compound is represented by Formula (I) or a solvate or pharmaceutically acceptable salt thereof, wherein —NR²B is selected from

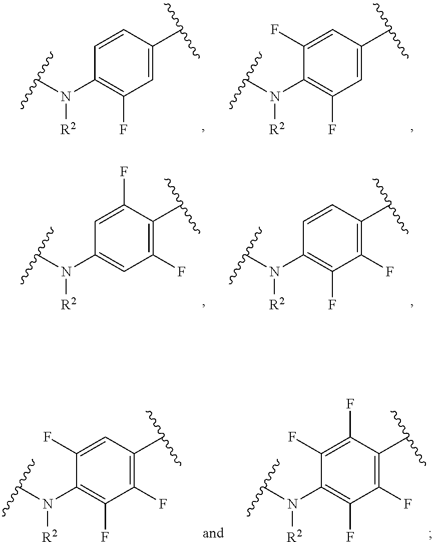

and wherein the residue —NR² on ring B is in a 1,4-orientation with respect to ring C.

In an upmost particular embodiment, the compound is represented by Formula (I) or a solvate or pharmaceutically acceptable salt thereof, wherein —NR²B is

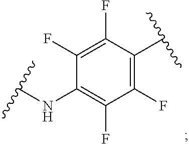 [SMILES: *Nc1c(F)c(F)c(*)c(F)c1F]

and wherein the residue —NR² on ring B is in a 1,4-orientation with respect to ring C.

In a more particular embodiment, the compound is represented by Formula (I) or a solvate or pharmaceutically acceptable salt thereof, wherein

C is phenyl, pyridyl or thiazolyl, wherein phenyl, pyridyl or thiazolyl is unsubstituted or substituted with 1 to 4 substituents independently selected from the group consisting of D, F, Cl, —CN, C₁₋₄-alkyl, fluoro-C₁₋₄-alkyl, O—C₁₋₄-alkyl and O-fluoro-C₁₋₄-alkyl, wherein alkyl having one or more hydrogen atoms optionally replaced by deuterium;
X is selected from D, F, Cl, —CN, C₁₋₄-alkyl, fluoro-C₁₋₄-alkyl, O—C₁₋₄-alkyl and O-fluoro-C₁₋₄-alkyl, wherein alkyl having one or more hydrogen atoms optionally replaced by deuterium.

In a more particular embodiment, the compound is represented by Formula (I) or a solvate or pharmaceutically acceptable salt thereof, wherein

C is phenyl, wherein phenyl is unsubstituted or substituted with 1 to 4 substituents independently selected from the group consisting of D, F, Cl, —CN, Me, CD₃, CHF₂, CF₃, —OMe, —OCD₃, —OCHF₂ and —OCF₃;
X is selected from D, F, Cl, —CN, Me, CD₃, CHF₂, CF₃, Et, CD₂CD₃, —OMe, —OCD₃, —OCHF₂, —OCF₃, —OEt and —OCD₂CD₃.

In one particular embodiment,

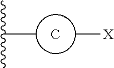

is selected from and

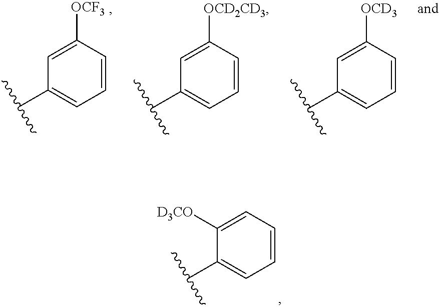

wherein the ring C is optionally substituted with 1 to 4 substituents selected from D and F.

In one particular embodiment,

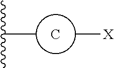

is selected from

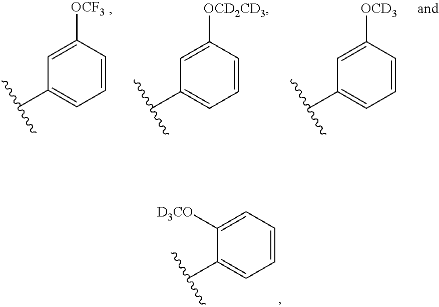

In a more particular embodiment, the compound is represented by Formula (I) or a solvate or pharmaceutically acceptable salt thereof, wherein

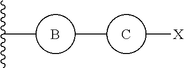

is selected from

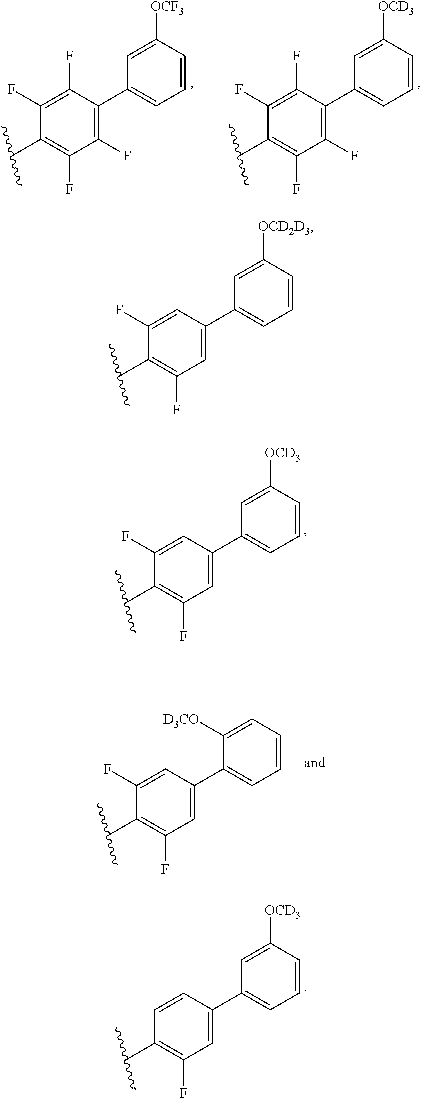

In a particular embodiment, the compound is represented by Formula (I) or a solvate or pharmaceutically acceptable salt thereof, wherein

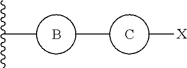

is selected from

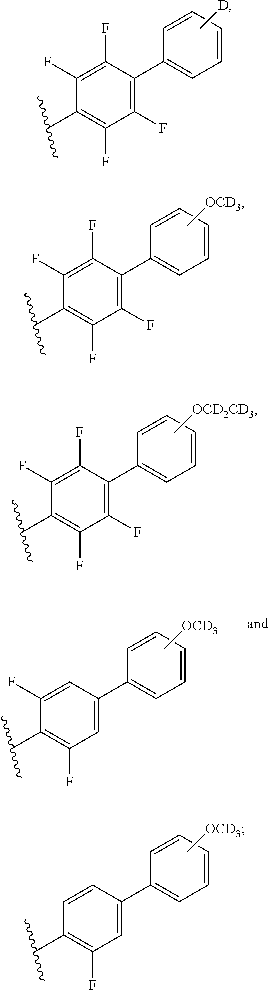

wherein ring C is optionally substituted with 1 to 4 substituents independently selected from D or F.

In a particular embodiment, the compound is represented by Formula (I) or a solvate or pharmaceutically acceptable salt thereof, wherein

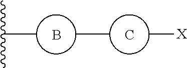

is selected from

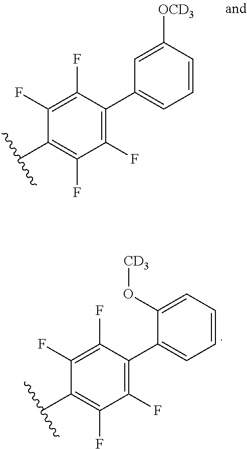

In one particular embodiment, the compound is represented by Formula (I) or a solvate or pharmaceutically acceptable salt thereof, wherein

Y is selected from

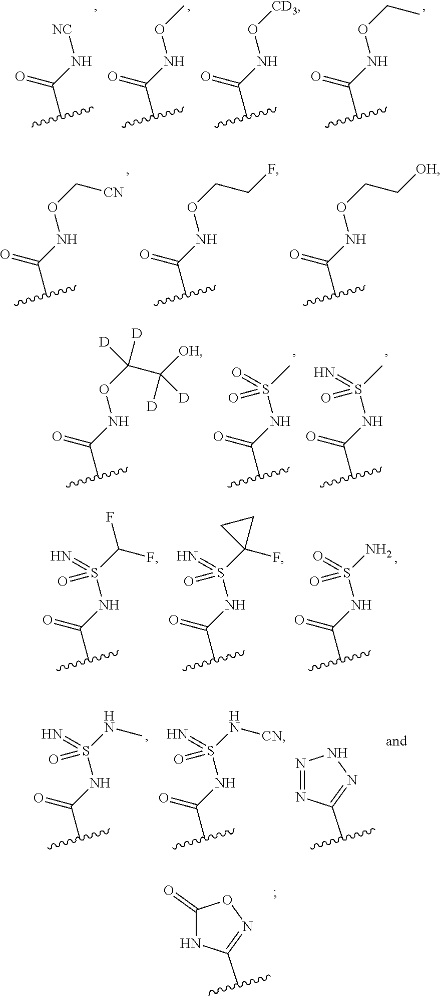

is selected from

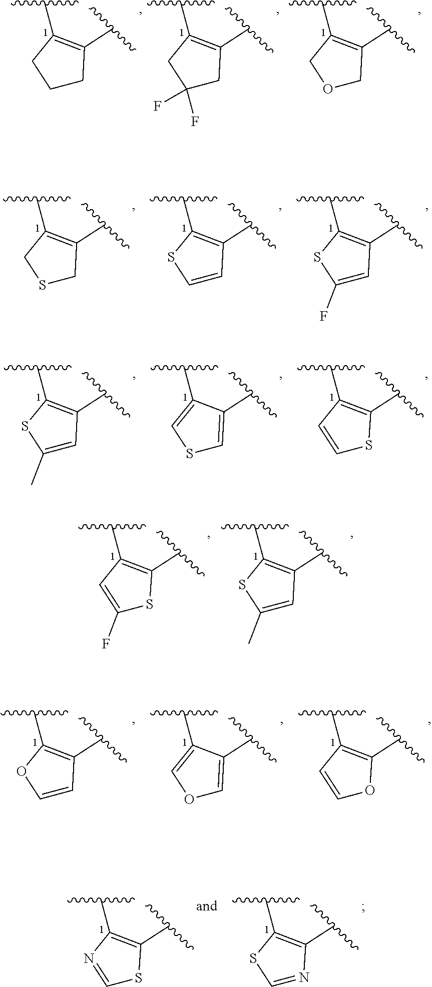

### R² is H;

B is selected from

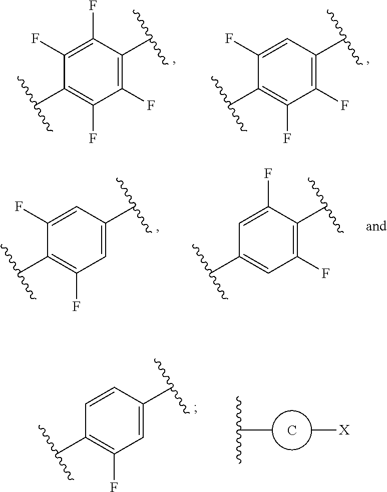

is selected from

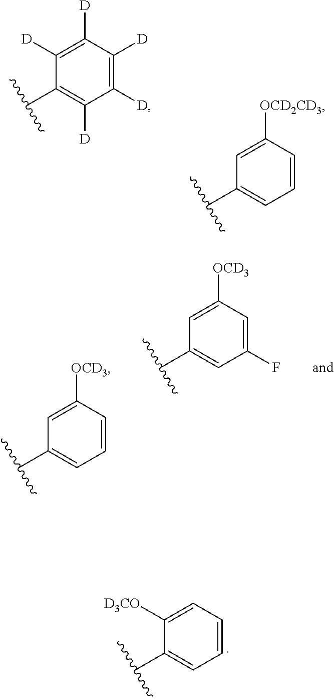

In an even more particular embodiment, the compound is represented by Formula (I) or a solvate or pharmaceutically acceptable salt thereof, wherein

Y is selected from

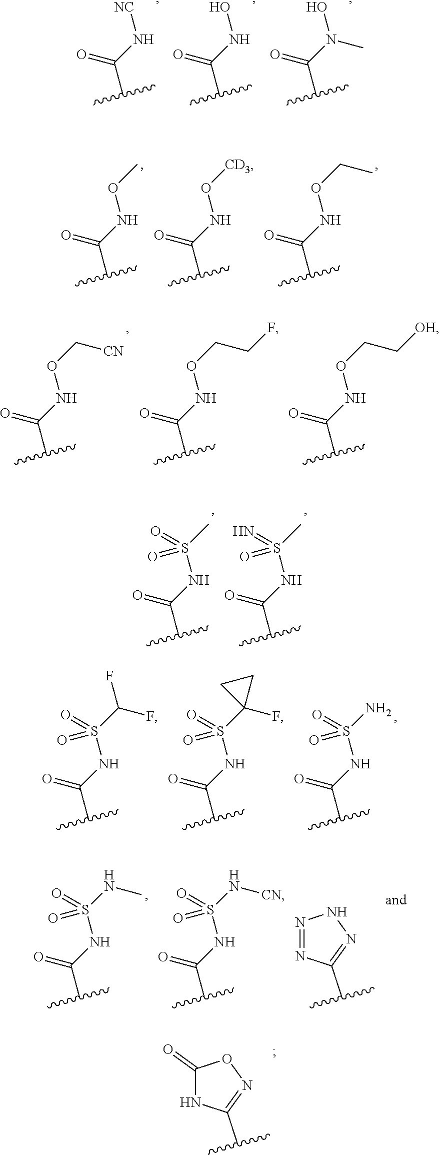

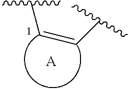

is selected from

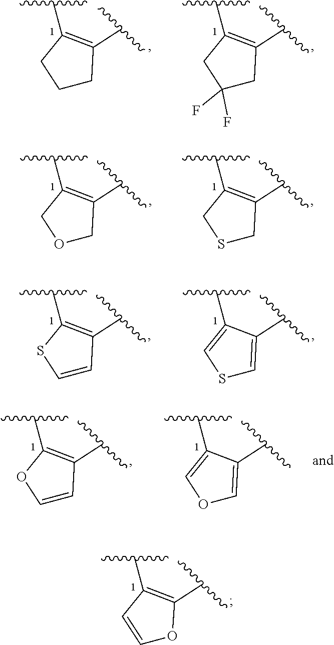

### R² is H;

B is selected from

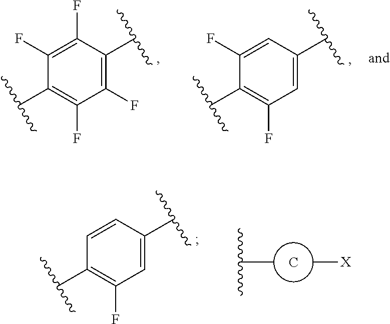

is selected from

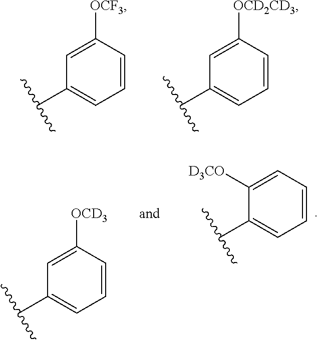

In an even more particular embodiment, the compound is represented by Formula (I) or a solvate or pharmaceutically acceptable salt thereof, wherein

Y is selected from

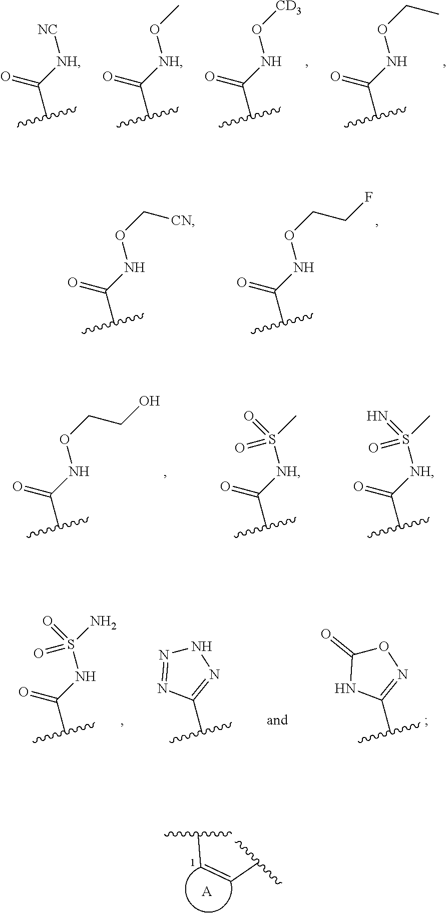

is selected from

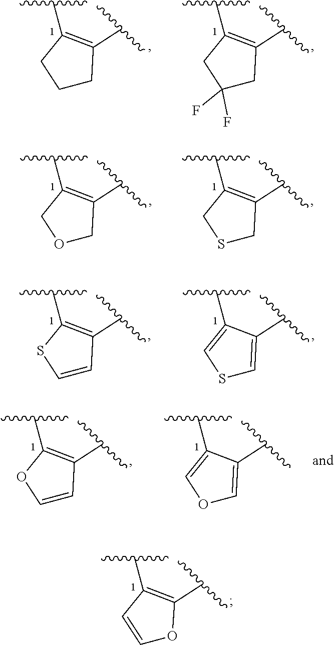

### R² is H;

B is selected from

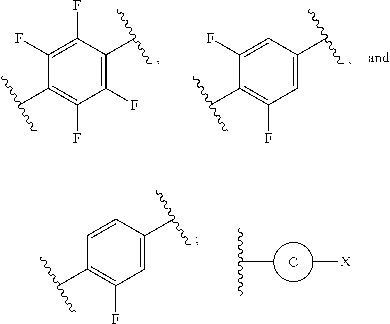

is selected from

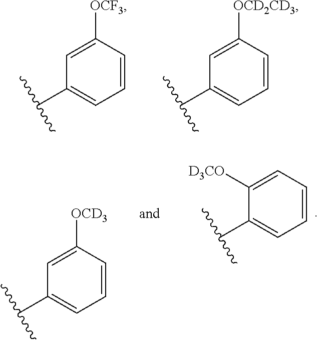

In an equally even more particular embodiment, the compound is represented by Formula (I) or a solvate or pharmaceutically acceptable salt thereof, wherein

Y is selected from

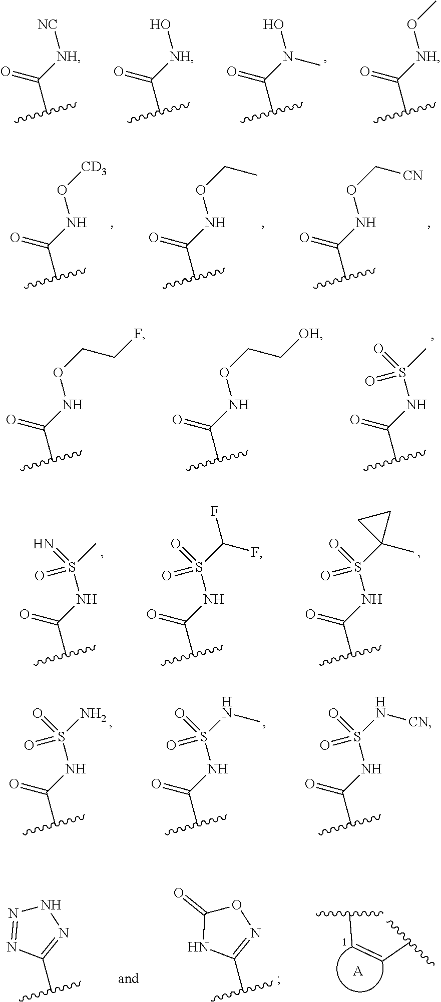

is selected from

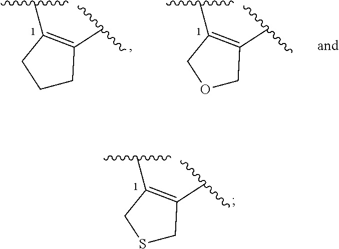 [SMILES: *C1=C(*)CCC1 ; *C1=C(*)COC1 ; *C1=C(*)CSC1]

### R² is H;

B is selected from

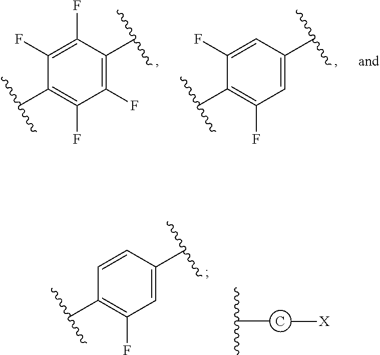

is selected from

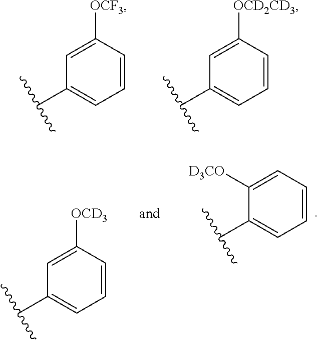

In an equally even more particular embodiment, the compound is represented by Formula (I) or a solvate or pharmaceutically acceptable salt thereof, wherein

Y is selected from

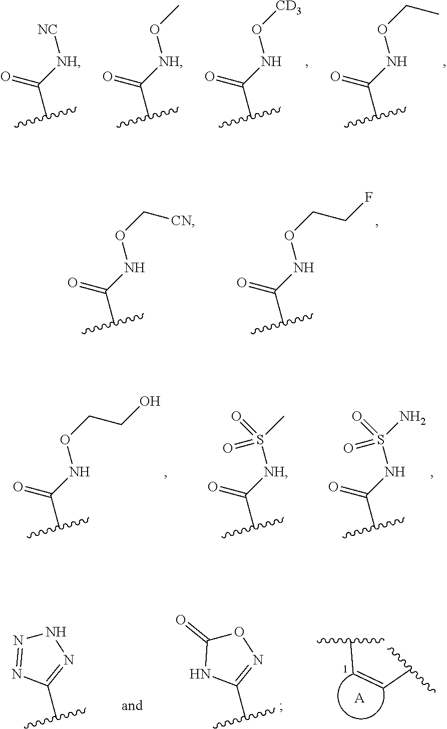

is selected from

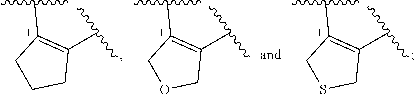 [SMILES: *C1=C(*)CCC1 ; *C1=C(*)COC1 ; *C1=C(*)CSC1]

### R² is H;

B is selected from

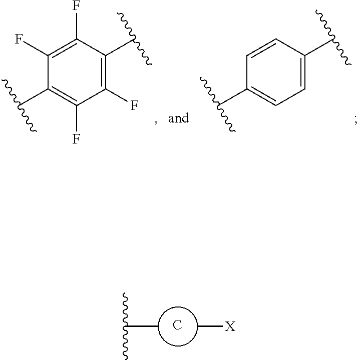

is selected from

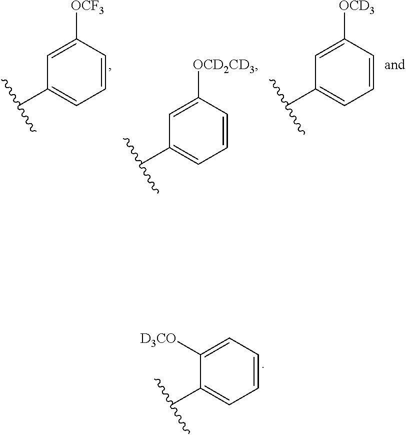

In an equally even more particular embodiment, the compound is represented by Formula (I) or a solvate or pharmaceutically acceptable salt thereof, wherein

Y is selected from

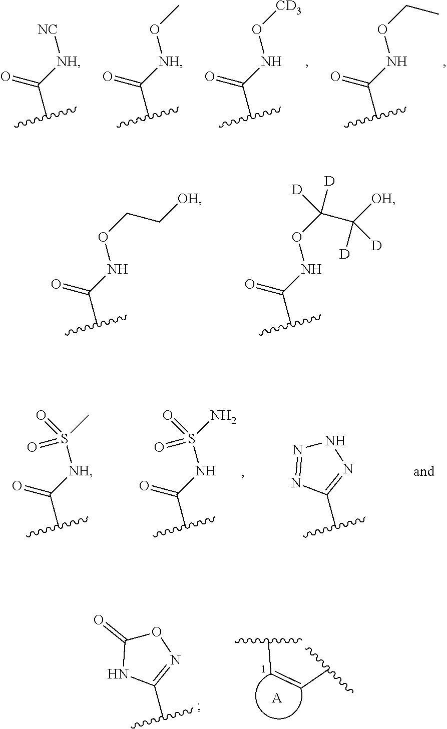

is selected from

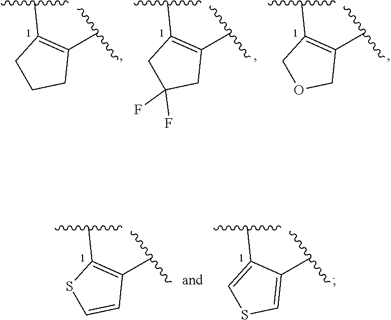 [SMILES: *C1=C(*)CCC1 ; *C1=C(*)CC(F)(F)C1 ; *C1=C(*)COC1 ; *c1ccsc1* ; *c1cscc1*]

### R² is H;

B is selected from

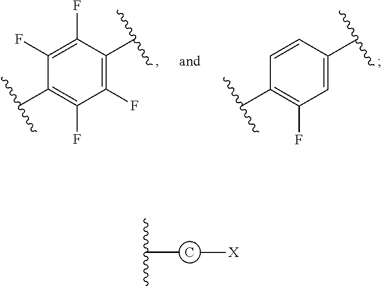

is selected from and

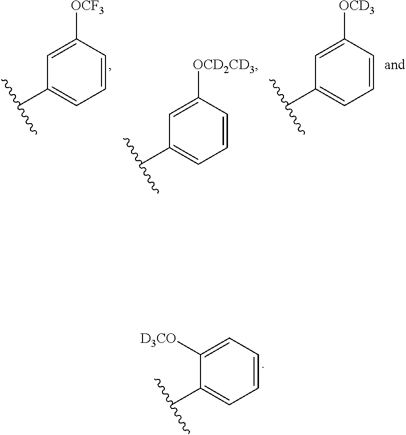

In a particular embodiment, the compound or a solvate or pharmaceutically acceptable salt thereof is selected from the Examples shown in the Experimental Part.

In a most particular embodiment, the compound or a solvate or pharmaceutically acceptable salt thereof is selected from

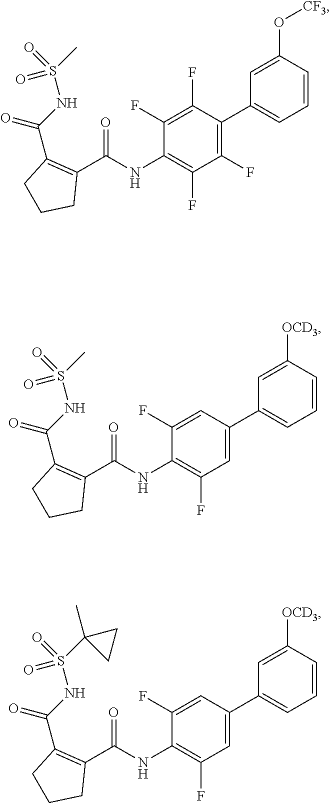

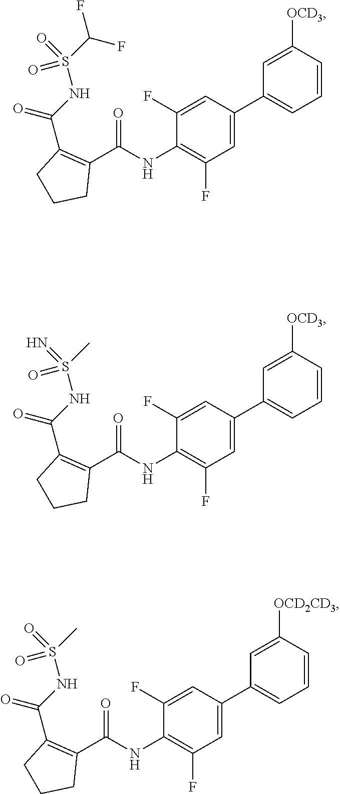

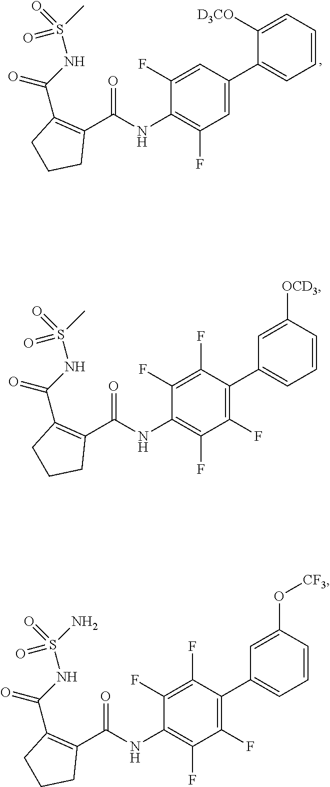

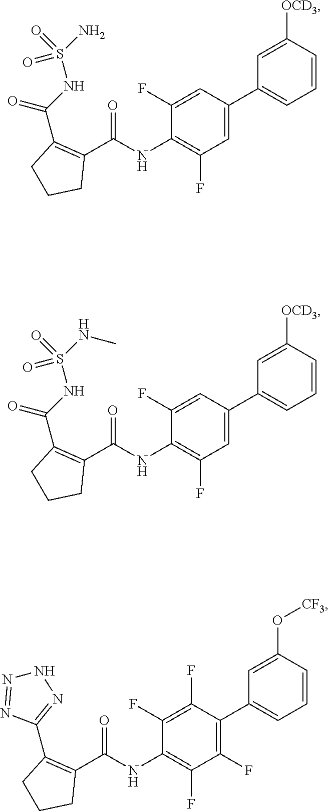

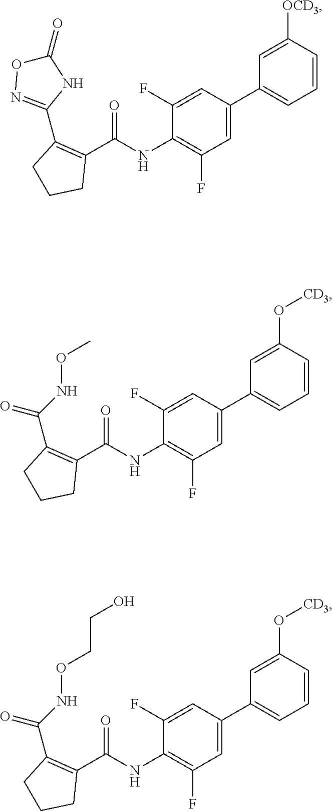

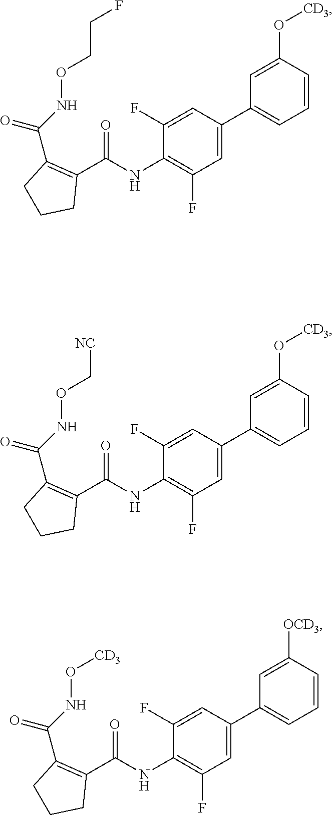

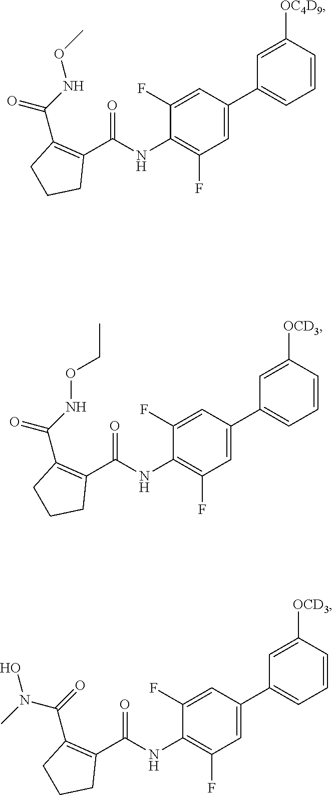

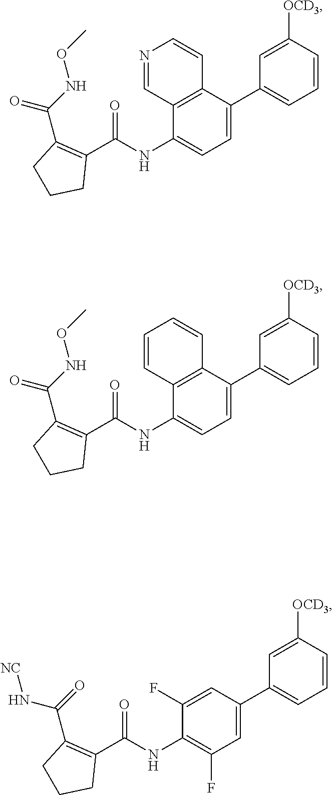

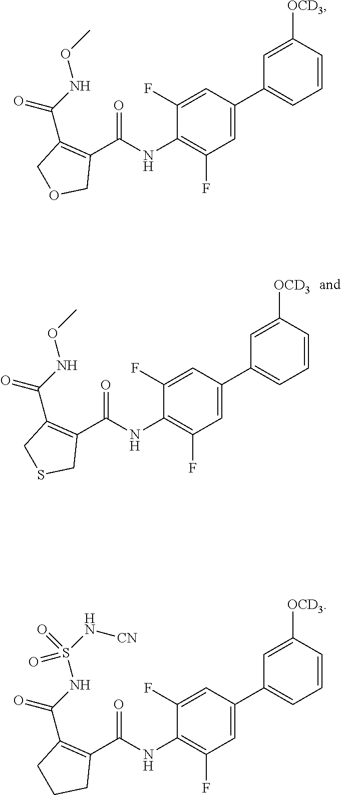

In an equally most particular embodiment, the compound or a solvate or pharmaceutically acceptable salt thereof is selected from

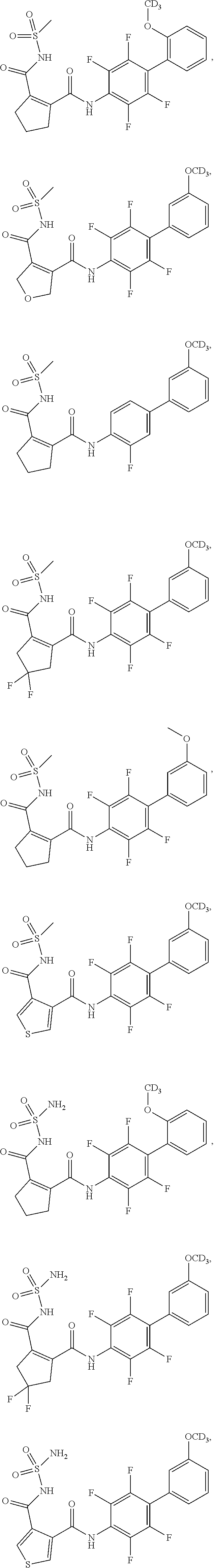

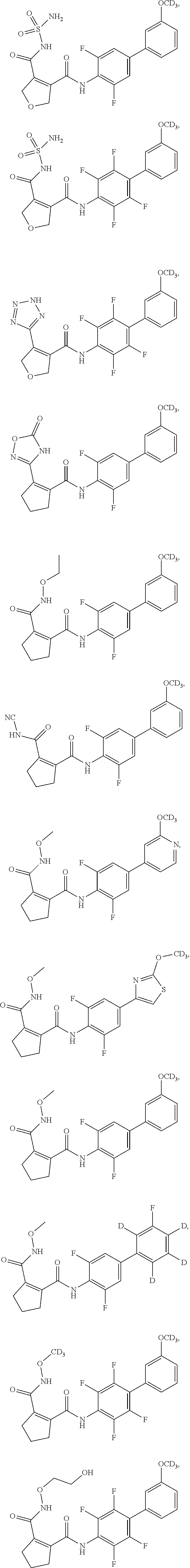

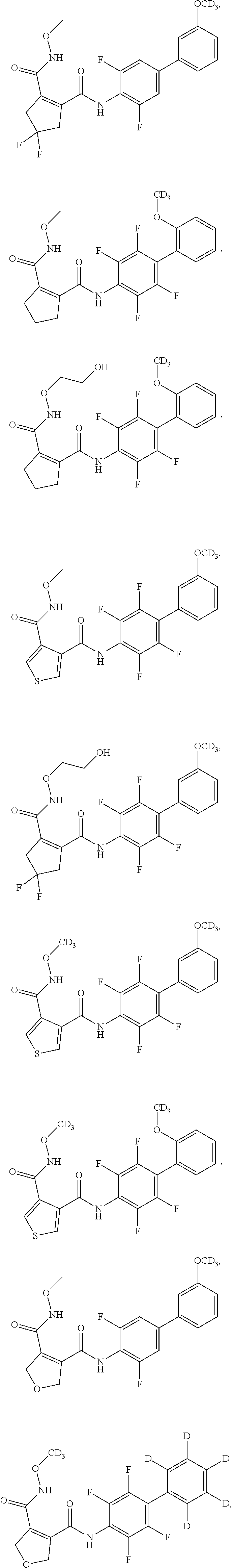

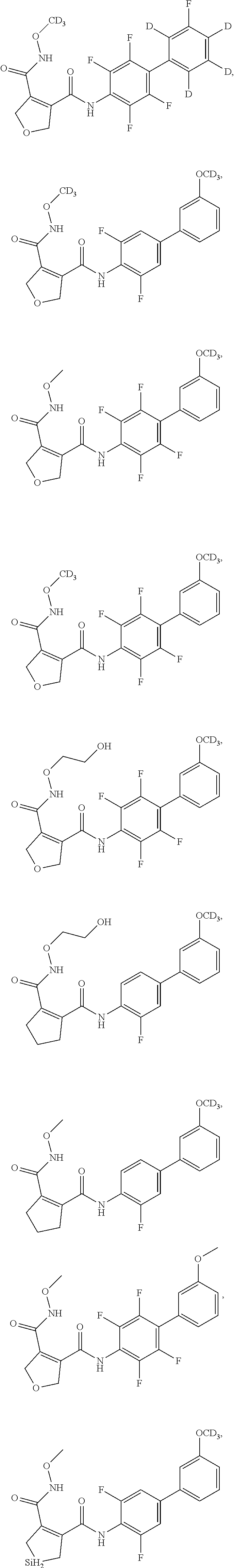

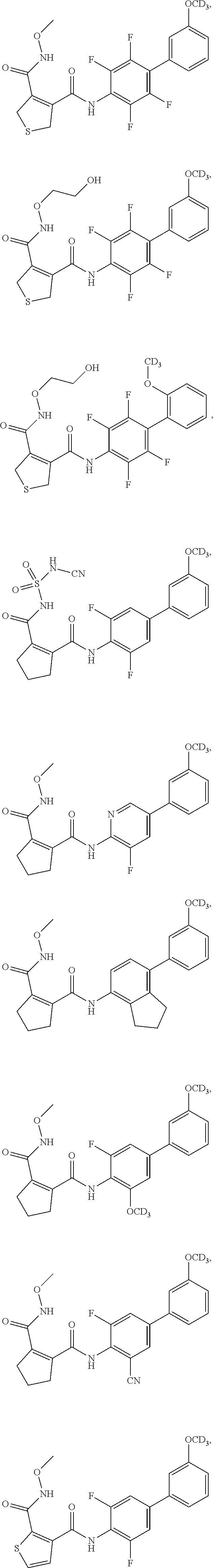

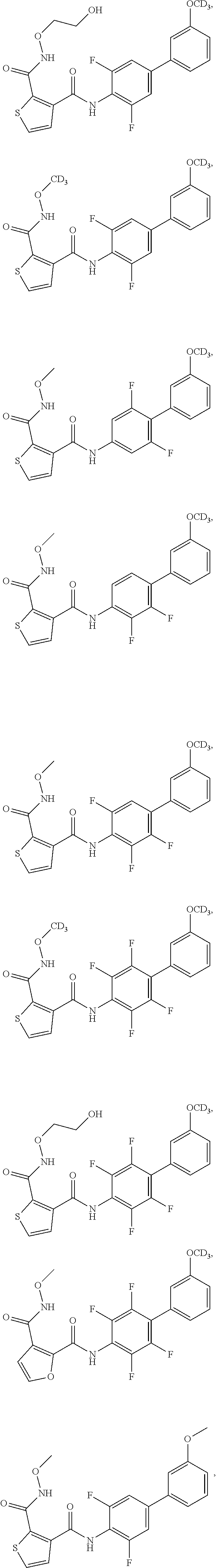

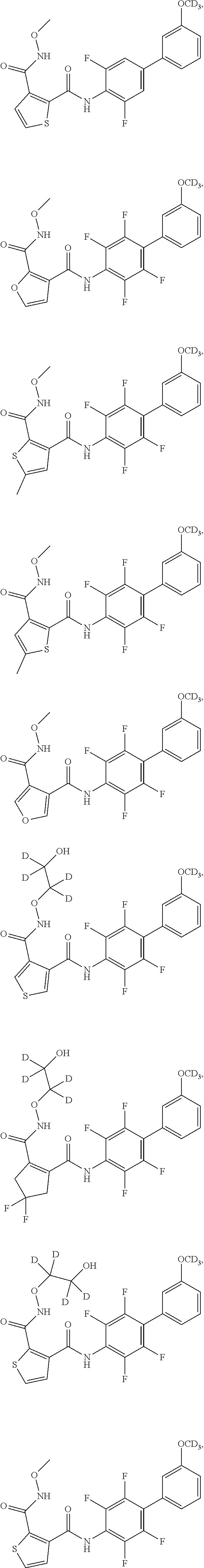

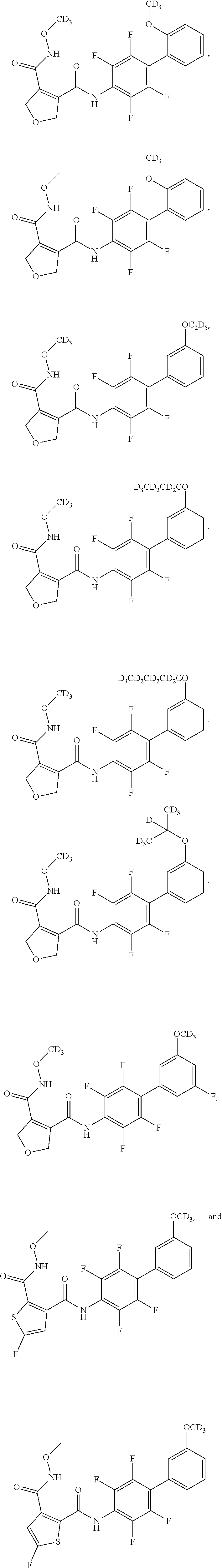

The invention also relates to the compound according to any of the preceding embodiments for the use as a medicament.

The invention also relates to the compound according to any of the preceding embodiments for use in the prophylaxis and/or treatment of diseases, disorders, therapeutic indications or medical conditions amenable for treatment with DHODH inhibitors.

The invention also relates to the compound according to any of the preceding embodiments for use in the prophylaxis and/or treatment of a DHODH mediated disease selected from rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.

More specifically, the invention relates to a compound according to any of the preceding embodiments for use wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

Also provided is a pharmaceutical composition comprising a compound of the present invention and a pharmaceutically acceptable carrier or excipient.

Also provided is a pharmaceutical composition comprising a compound of the present invention and a pharmaceutically acceptable carrier or excipient ands further comprising one or more additional therapeutic agents selected from anti-inflammatory agents, anti-viral agents, immunosuppressive and/or immunomodulatory agents, steroids, non-steroidal anti-inflammatory agents, antihistamines, analgesics and suitable mixtures thereof. The term “pharmaceutically acceptable carrier” as used herein indicates that the carrier is approved or recognized for use in animals, and more particularly in humans, i.e. it is not toxic to the host or patient. In addition a carrier of choice will not interfere with the effectiveness of the biological activity of the active ingredient. The term “carrier” refers to any auxiliary material necessary for the particular mode of administration of choice and includes e.g. solvents, diluents, excipients or other additives with which the compound of the invention is administered. Typically used diluents pharmaceutical carriers include sterile liquids, such as aqueous solutions and oils (e.g. of petroleum, animal, vegetable or synthetic origin), e.g. peanut oil, soybean oil, mineral oil, sesame oil and the like. Typically used aqueous liquids include water, saline solutions, aqueous dextrose and glycerol solutions and the like. Suitable pharmaceutical excipients include citric acid, ascorbic acid, starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. Optionally the composition may comprise additives, such as wetting or emulsifying agents, pH buffering agents or binders. Examples of suitable pharmaceutical carriers are well known in the art and are described in e.g. “Remington's Pharmaceutical Sciences” by E. W. Martin (18th ed., Mack Publishing Co., Easton, PA (1990).

According to expert's knowledge the compounds of the invention as well as their salts may contain, e.g. when isolated in crystalline form, varying amounts of solvents. Included within the scope of the invention are therefore all solvates and in particular all hydrates of the compounds of Formula (I) as well as all solvates and in particular all hydrates of the salts of the compounds of Formula (I).

The present invention further relates to methods of prophylaxis and/or treatment of diseases, disorders, therapeutic indications or medical conditions which are described herein, particularly a disease or medical condition in which the inhibition of DHODH is beneficial, more particularly a disease or medical condition selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy, said method comprising administering to a subject in need thereof an effective amount of a compound of Formula (I) as described herein. Analogously, the present invention further relates to methods as the one described above, which encompass the further embodiments described herein, in particular the medical uses and compounds for use in medical treatments as described herein.

The present invention further relates to methods of prophylaxis and/or treatment of diseases, disorders, therapeutic indications or medical conditions which are described herein, particularly a disease or medical condition in which the inhibition of DHODH is beneficial, more particularly a disease or medical condition selected from graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis, said method comprising administering to a subject in need thereof an effective amount of a compound of Formula (I) as described herein.

The present invention further relates to pharmaceutical compositions, kits and kits-of parts comprising the compounds according to the present invention.

The present invention further relates to the use of the compounds according to the present invention for the production of pharmaceutical compositions which are employed for the treatment and/or prophylaxis of the diseases, disorders, illnesses and/or conditions as mentioned herein.

The present invention further relates to the methods and medical uses described herein, encompassing the pharmaceutical compositions as described herein.

The pharmaceutical compositions as described herein comprise one or more of the compounds according to this invention and a pharmaceutically acceptable carrier or excipient.

The pharmaceutical compositions as described herein comprise one or more of the compounds according to this invention and a pharmaceutically acceptable carrier or excipient, further comprising one or more additional therapeutic agents selected from anti-inflammatory agents, anti-viral agents, immunosuppressive and/or immunomodulatory agents, steroids, non-steroidal anti-inflammatory agents, antihistamines, analgesics and suitable mixtures thereof.

Additionally, the invention relates to an article of manufacture, which comprises packaging material and a pharmaceutical agent contained within said packaging material, wherein the pharmaceutical agent is therapeutically effective against the medical conditions as described herein, and wherein the packaging material comprises a label or package insert which indicates that the pharmaceutical agent is useful for preventing or treating said medical conditions, and wherein said pharmaceutical agent comprises one or more compounds of Formula (I) according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or particularly in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

Depending upon the particular disease, to be treated or prevented, additional therapeutic active agents, which are normally administered to treat or prevent that disease, may optionally be coadministered with the compounds according to the present invention. As used herein, additional therapeutic agents that are normally administered to treat or prevent a particular disease are known as appropriate for the disease being treated.

In a further aspect of the present invention, the compounds according to this invention or the salts or solvates of said compounds of Formula (I) may be combined with standard therapeutic agents which are commonly used for the treatment of the medical conditions as described herein.

The person skilled in the art is aware on the base of his/her expert knowledge of the total daily dosage(s) and administration form(s) of the additional therapeutic agent(s) coadministered. Said total daily dosage(s) can vary within a wide range. In practicing the present invention and depending on the details, characteristics or purposes of their uses mentioned above, the compounds according to the present invention may be administered in combination therapy separately, sequentially, simultaneously or chronologically staggered (e.g. as combined unit dosage forms, as separate unit dosage forms or a adjacent discrete unit dosage forms, as fixed or nonfixed combinations, as kit-of-parts or as admixtures) with one or more standard therapeutics, in particular art-known chemotherapeutic or target specific anti-cancer agents, such as those mentioned above.

Thus, a further aspect of the present invention is a combination or pharmaceutical composition comprising a first active ingredient, which is a compound according to this invention or a pharmaceutically acceptable salt or solvate thereof, a second active ingredient, which is an art-known standard therapeutic for the medical conditions as described herein, and optionally a pharmacologically acceptable carrier, diluent and/or excipient for sequential, separate, simultaneous or chronologically staggered use in therapy in any order, e.g. to treat, prevent or ameliorate in a patient the medical conditions as described herein. In this context, the present invention further relates to a combination comprising a first active ingredient, which is at least one compound according to this invention, and a second active ingredient, which is at least one art-known standard therapeutic for the medical conditions as described herein, for separate, sequential, simultaneous or chronologically staggered use in therapy, such as e.g. in therapy of those diseases mentioned herein.

The term “combination” according to this invention may be present as a fixed combination, a non-fixed combination or a kit-of-parts. A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

The first and second active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy. The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration. The amounts of the first and second active ingredients of the combinations, compositions or kits according to this invention may together comprise a therapeutically effective amount for the treatment, prophylaxis or amelioration of a medical condition as described herein

A further aspect of the present invention is a method for treating cotherapeutically the medical conditions as described herein, in a patient in need of such treatment comprising administering separately, sequentially, simultaneously, fixed or non-fixed a therapeutically effective and tolerable amount of one or more of the compounds according to the present invention and a therapeutically effective and tolerable amount of one or more art-known therapeutic agents for the medical conditions as described herein, to said patient.

References and claims to the use of a compound of the Formula (I) or a pharmaceutically acceptable salt or solvate thereof in the manufacture of a medicament for the treatment of a disease or medical condition in their general and specific forms likewise refer to the corresponding methods of treating said disease or medical condition, said method comprising administering a therapeutically effective and tolerable amount of a compound of the Formula (I) or a pharmaceutically acceptable salt or solvate thereof to a subject in need thereof, compositions comprising a compound of the Formula (I) or a pharmaceutically acceptable salt or solvate thereof for the treatment of said disease or medical condition, a compound of the Formula (I) or a pharmaceutically acceptable salt or solvate thereof for use in the treatment of said disease or medical condition, and vice versa.

For the production of the pharmaceutical compositions, the compounds of the invention (=active compounds) are particularly mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions. The pharmaceutical compositions according to the invention are prepared by processes known per se.

The dosage of the active compounds is carried out in the customary order of magnitude. Topical application forms (such as ointments) thus contain the active compounds in a concentration of, for example, 0.1 to 99%. The customary dose in the case of systemic therapy (p.o.) is usually between 0.3 and 30 mg/kg per day, (i.v.) is usually between 0.3 and 30 mg kg/h. The choice of the optimal dosage regime and duration of medication, particularly the optimal dose and manner of administration of the active compounds necessary in each case can be determined by a person skilled in the art on the basis of his/her expert knowledge.

The class of compounds of the present invention is useful for the development of medicaments suitable for the treatment of autoimmune or viral diseases and chronic inflammation or, more generally, for the treatment of diseases where the inhibition of DHODH is beneficial. The compounds of the present invention are also useful for the treatment of diseases such as rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy. More specifically, the disease is selected graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

The class of compounds of the present invention is useful for the treatment of viral diseases, especially acute viral infections selected from Coronavirus infections, COVID-19, SARS, flu/influenza (and avian influenza), HIV/Aids, chickenpox (Varicella), cytomegalovirus, Dengue Fever, German measles (Rubella), hand-foot-mouth disease, hantavirus infections, all forms of hepatitis, Lassa fever, Marburg virus infections, measles, meningitis, MERS-CoV, mumps, norovirus infections, herpes simplex virus infections, smallpox, rotavirus infections, Ebola virus, poliovirus infections, rhinovirus infections, parainflunenzavirus infections, RSV infections, HCMV infections and bannavirus infections. Most preferred as COVID-19, flu/influenza and rhinovirus infections, most preferred is COVID-19. It is understood, that also mutated forms of the virus (e.g. of SARS-CoV-2) are covered.

### Combination or Alternation Therapy

The compounds or their pharmaceutically acceptable salts as described herein can be administered on top of the current standard of care for patients, or in combination or alternation with any other compound or therapy that the healthcare provider deems beneficial for the patient. The combination and/or alternation therapy can be therapeutic, adjunctive or palliative.

Especially preferred are is a combination or alternation therapy for the treatment of anti-viral infections, especially Covid-19:

It has been observed that high levels of the cytokine interleukin-6 (IL-6) are a precursor to respiratory failure and death in COVID-19 patients. To treat this surge of an immune response, which may constitute a cytokine storm, patients can be administered an IL-6-targeting monoclonal antibody, pharmaceutical inhibitor or protein degrader such as a bispecific compound that binds to IL-6 and also to a protein that mediates degradation. Examples of antibodies include tocilizumab, sarilumab, siltuximab, olokizumab and clazakizumab. In one embodiment, a compound of Formula (I) or a pharmaceutically acceptable salt thereof is administered in combination or in alternation with tocilizumab or sarilumab. Additional nonlimiting examples of immunosuppressant drugs used to treat the overreacting immune system include Janus kinase inhibitors (tofacitinib, baricitinib, filgotinib); calcineurin inhibitors (cyclosporine), tacrolimus, mTOR inhibitors (sirolimus, everolimus) and IMDH inhibitors (azathioprine). Additional antibodies and biologics include abatacept, adalimumab, anakinra, certolizumab, etanercept, golimumab, infliximab, ixekizumab, natalizumab, rituximab, secukinumab, tocilizumab, ustekinumab, vedolizumab, basiliximab and daclizumab.

IL-1 blocks the production of IL-6 and other proinflammatory cytokines. COVID patients are also sometimes treated with anti-IL-1 therapy to reduce a hyperinflammatory response, for example, an intravenous administration of anakinra. Anti-IL-1 therapy generally may be for example, a targeting monoclonal antibody, pharmaceutical inhibitor or protein degrader such as a bispecific compound that binds to IL-1 and also to a protein that mediates degradation.

Patients with COVID often develop viral pneumonia, which can lead to bacterial pneumonia. Patients with severe COVID-19 can also be affected by sepsis or “septic shock”. Treatment for bacterial pneumonia secondary to COVID or for sepsis includes the administration of antibiotics, for example a macrolide antibiotic, including azithromycin, clarithromycin, erythromycin, or roxithromycin.

Additional antibiotics include amoxicillin, doxycycline, cephalexin, ciprofloxacin, clindamycin, metronidazole, sulfamethoxazole, trimethoprim, amoxicillin, clavulanate or levofloxacin. In one embodiment, thus a compound of Formula (I) or a pharmaceutically acceptable salt thereof is administered in combination or in alternation with an antibiotic, for example, azithromycin. Some of these antibiotics such as azithromycin have independent anti-inflammatory properties. Such drugs may be used both as anti-inflammatory agents for COVID patients and have a treatment effect on secondary bacterial infections.

A unique challenge in treating patients infected with COVID-19 is the relatively long-term need for sedation if patients require mechanical ventilation which might last up to or greater than 5, 10 or even 14 days. For ongoing pain during this treatment, analgesics can be added sequentially and for ongoing anxiety, sedatives can be added sequentially. Non-limiting examples of analgesics include acetaminophen, ketamine and PRN opioids (hydromorphone, fentanyl, and morphine). Non-limiting examples of sedatives include melatonin, atypical antipsychotics with sedative-predominant properties (olanzapine, quetiapine), propofol or dexmedetomidine, haloperidol and phenobarbital. In one embodiment, a compound of Formula (I) or a pharmaceutically acceptable salt, a solvate, a solvate of a salt, a hydrate or a polymorph thereof is administered in combination or in alternation with a pain reliever, such as acetaminophen, ketamine, hydromorphone, fentanyl, or morphine. In one embodiment, a compound of Formula (I) a pharmaceutically acceptable salt, a solvate, a solvate of a salt, a hydrate or a polymorph thereof is administered in combination or in alternation with a sedative, such as melatonin, olanzapine, quetiapine, propofol, dexmedetomidine, haloperidol or phenobarbital.

In one embodiment, a compound of the present invention is used in an effective amount in combination with a protease inhibitor such as PF-07304814, PF-00835231, PF-07321332 (nirmatrelvir), lopinavir or ritonavir. In one more special embodiment, protease inhibitor is PF-07321332 (nirmatrelvir).

In one embodiment, a compound of the present invention is used in an effective amount in combination with a RNA replication modulator such as N4-hydroxycytidine or a prodrug thereof may also be administered. In one special embodiment, the RNA replication modulator is a N4-hydroxycytidine prodrug as described in WO 2019/113462. In one more special embodiment, the RNA replication modulator is molnupiravir.

In one embodiment, a compound of the present invention is used in an effective amount in combination with halofuginol or an enantiomer, tautomer, solvate or pharmaceutically acceptable salt thereof.

In one embodiment, a compound of the present invention is used in an effective amount in combination with dipyridamole or a solvate or pharmaceutically acceptable salt thereof.

In one embodiment, a compound of the present invention is used in an effective amount in combination with gemcitabine or a solvate or pharmaceutically acceptable salt thereof.

In one embodiment, a compound of the present invention is used in an effective amount in combination with AT-527 (RO7496998) or a solvate or pharmaceutically acceptable salt thereof.

Additional drugs that may be used in the treatment of a COVID patient include, but are not limited to aspirin, colchicine, dimethyl fumarate, acalabrutinib, favipiravir, fingolimod, methylprednisolone, bevacizumab, tocilizumab, umifenovir, losartan and the monoclonal antibody combination of REGN3048 and REGN3051 or ribavirin. Any of these drugs or vaccines can be used in combination or alternation with an active compound provided herein to treat a viral infection susceptible to such.

In one embodiment, a compound of the present invention is used in an effective amount in combination with anti-coronavirus vaccine therapy, including but not limited to mRNA-1273 (Moderna), AZD-1222 (AstraZeneca and University of Oxford), BNT162b2 (BioNTech), CoronaVac (Sinovac), NVX-CoV 2372 (NovoVax), SCB-2019 (Sanofi and GSK), ZyCoV-D (Zydus Cadila) and CoVaxin (Bharat Biotech). In another embodiment, a compound of the present invention is used in an effective amount in combination with passive antibody therapy or convalescent plasma therapy.

SARS-CoV-2 is constantly mutating, which many increase virulence and transmission rates. Drug-resistant variants of viruses may emerge after prolonged treatment with an antiviral agent. Drug resistance may occur by mutation of a gene that encodes for an enzyme used in viral replication. The efficacy of a drug against an RNA virus infection in certain cases can be prolonged, augmented or restored by administering the compound in combination or alternation with another and perhaps even two or three other, antiviral compounds that induce a different mutation or act through a different pathway, from that of the principle drug. A variant of a known virus can refer to a virus carrying one or more nucleotide mutations in the viral genome as compared to the known virus, for instance at least 1, 2, 3, 4, 5, 6, 7, 8, 9, 10, 15, 20, 30, 40, 60, 100, 200, 300 or even more nucleotide mutations. Mutations can refer to nucleotide deletion, insertion, or substitution. In some cases, a variant can have at most 50%, 40%, 30%, 20%, 10%, 5%, 4%, 3%, 2% or 1% of the viral genome different than the genome of a known virus.

Alternatively, the pharmacokinetics, biodistribution, half-life or other parameter of the drug can be altered by such combination therapy (which may include alternation therapy if considered concerted). Examples of other therapeutic agents that may be combined with a compound of Formula (I) or a pharmaceutically acceptable salt, a solvate, a solvate of a salt, a hydrate or a polymorph thereof, either administered separately, or in the same pharmaceutical composition include, but are not limited to a:
(1) Protease inhibitor;
(2) Polymerase inhibitor (e.g. gemcitabine);
(3) Allosteric polymerase inhibitor;
(4) Interferon alfa-2a, which may be pegylated or otherwise modified, and/or ribavirin;
(5) Non-substrate-based inhibitor;
(6) Helicase inhibitor;
(7) Primase-helicase inhibitor;
(8) Antisense oligodeoxynucleotide (S-ODN);
(9) Aptamer;
(10) Nuclease-resistant ribozyme;
(11) iRNA, including microRNA and SiRNA;
(12) Antibody, partial antibody or domain antibody to the virus;
(13) Viral antigen or partial antigen that induces a host antibody response;
(14) NOD-, LRR- and pyrin domain-containing protein 3 (NLRP3);
(15) Glutamyl-prolyl-tRNA synthetase inhibitor (e.g. halofuginone);
(16) Equilibrative nucleoside transporter (ENT) inhibitor (e.g. dipyridamole);
(17) other DHODH inhibitors (e.g. brequinar, teriflunomide, leflunomide, PTC299, MEDS433, AG-636, ASLAN003, JNJ-74856665, RP7214, PP-001 and BAY2402234).

It will be recognized that some variation of natural isotopic abundance occurs in a synthesized compound depending upon the origin of chemical materials used in the synthesis. Thus, a preparation of vidofludimus and compounds according Formula (I) without any depicted deuterium will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Comp. Biochem. Physiol. 1998; 119A:725.

The term “isotopic enrichment factor” at a particular position normally occupied by hydrogen refers to the ratio between the abundance of deuterium at the position and the natural abundance of deuterium at that position. By way of example, an isotopic enrichment factor of 3500 means that the amount of deuterium at the particular position is 3500-fold the natural abundance of deuterium, or that 52.5% of the compounds have deuterium at the particular position (i.e., 52.5% deuterium incorporation at the given position). The abundance of deuterium in the oceans of Earth is approximately one atom in 6500 hydrogen atoms (about 154 parts per million (ppm)). Deuterium thus accounts for approximately 0.015 percent (on a weight basis, 0.030 percent) of all naturally occurring hydrogen atoms in the oceans on Earth; the abundance changes slightly from one kind of natural water to another.

When a particular position in a compound of the invention (e.g., a compound represented by Formula (I) or a pharmaceutically acceptable salt and/or solvate thereof) is designated by name or structure as containing hydrogen or deuterium, it is to be understood that the position can contain hydrogen at its natural abundance or can be enriched in deuterium with an isotopic enrichment factor of, for example, at least 835 (12.5% deuterium incorporation), of at least 1670 (25% deuterium incorporation, of at least 3500 (52.5% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When a particular position in a compound of the invention (e.g., a compound represented by Formula (I) or a pharmaceutically acceptable salt and/or solvate thereof) is designated specifically by name or structure as “H” or “hydrogen”, the position is understood to have hydrogen at its natural abundance isotopic composition.

When a particular position in a compound of the invention (e.g., a compound represented by Formula (I) or a pharmaceutically acceptable salt and/or solvate thereof) is designated specifically by name or structure as “D” or “deuterium”, the position is understood to have deuterium at an abundance that is at least 3340 times of the natural abundance of deuterium, which is 0.015% (i.e., at least 50.1% incorporation of deuterium), at least 3500 times of the natural abundance of deuterium (52.5% deuterium incorporation), at least 4500 times of the natural abundance of deuterium (67.5% deuterium incorporation), at least 5000 (75% deuterium), at least 5500 times of the natural abundance of deuterium (82.5% deuterium incorporation), at least 6000 times of the natural abundance of deuterium (90% deuterium incorporation), at least 6333.3 times of the natural abundance of deuterium (95% deuterium incorporation), at least 6466.7 times of the natural abundance of deuterium (97% deuterium incorporation), at least 6600 times of the natural abundance of deuterium (99% deuterium incorporation), or at least 6633.3 times of the natural abundance of deuterium (99.5% deuterium incorporation).

The percentage of deuterium incorporation can be obtained by quantitative analysis using a number of conventional methods, such as mass spectroscopy (peak area) or by quantifying the remaining residual ¹H-NMR signals of the specific deuteration site compared to signals from internal standards or other, non-deuterated ¹H signals in the compound.

When a chemical name or structure is silent as to whether a particular position in a compound normally occupied by hydrogen is isotopically enriched, it is intended that the particular position is occupied by hydrogen at its natural abundance. By way of example, the term “phenyl” or

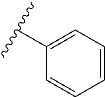 [SMILES: *c1ccccc1]

without any further designation as to isotopic enrichment indicates that all hydrogen atoms are present at natural abundance.

When ring A is a partially saturated cycle, the double bond in ring A is located in the depicted position:

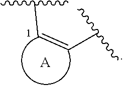

In case ring A is a 5-membered heteroaryl ring, then the double bond is within a delocated π-system and can exist in mesomeric forms. An example are the following thiophene mesomeric forms:

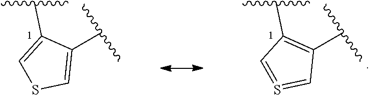 [SMILES: *c1cscc1* ; *C1=C(*)C=S=C1]

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

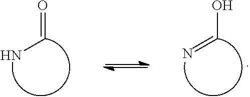

A cycloalkyl or heterocycloalkyl group can be connected straight or spirocyclic, e.g. when cyclohexane is substituted with the heterocycloalkyl group oxetane, the following structures are possible:

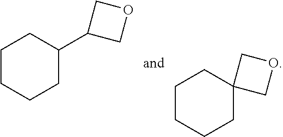 [SMILES: C1CCC(C2COC2)CC1 ; C1CCC2(CC1)COC2]

The term “1,4-orientation” (as mentioned for ring B) denotes the specific relative position of the two substituents on the same ring and means that on a ring the substituents have at least one possibility, where 4 atoms are between the two substituents in the ring attached to the ring system:

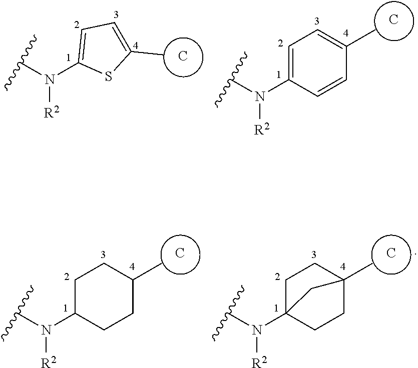

The term “1,3-orientation” means denotes the specific relative position of the two substituents on the same ring and that on a ring the substituents have at least one possibility, where 3 atoms are between the two substituents attached to the ring system, e.g.

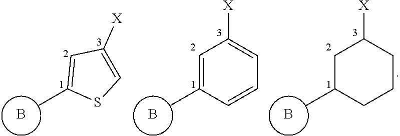

The term “compound,” when referring to any compound of this disclosure, including a compound represented by Formula (I) or a pharmaceutically acceptable salt and/or solvate thereof, refers to a collection of molecules having an identical chemical structure, except that there may be isotopic variation among the constituent hydrogen atoms of the molecules. The relative amount of isotopic variation in a compound of this invention will depend upon a number of factors including the isotopic purity of deuterated reagents used to make the compound and the efficiency of incorporation of deuterium in the various synthesis steps used to prepare the compound.

“D” and “d” both refer to deuterium. “H” refers to hydrogen.

“Substituted with deuterium” refers to the replacement of one or more hydrogen atoms with a corresponding number of deuterium atoms.

Any formula or structure given herein, is also intended to represent deuterated compounds comprising in addition further isotopically labelled atoms. Examples of additional isotopes that can be incorporated into compounds of the disclosure include further isotopes of hydrogen (i.e. tritium or ³H), as well as isotopes of carbon, nitrogen, oxygen, phosphorous, fluorine and chlorine, such as, but not limited to ¹¹C, ¹³C, ¹⁴C, ¹⁵N, ¹⁸F, ³¹P, ³²P, ³⁵S, ³⁶Cl and ¹²⁵I. The disclosure further comprises various isotopically labelled compounds into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or radioactive treatment of patients.

Halogen is selected from fluorine, chlorine, bromine and iodine, more preferably fluorine or chlorine and most preferably fluorine.

In the context of the present invention “C₁₋₄-alkyl” means a preferably saturated hydrocarbon chain having 1 to 4 carbon atoms which may be straight chained or branched. Examples thereof include methyl, ethyl, propyl, isopropyl, n-butyl, isobutyl and tert-butyl. Preferred is C₁₋₃-alkyl, such as methyl, ethyl, propyl and isopropyl, most preferred is methyl. The term “alkyl” by itself or as a part of another substituent, e.g. halo-C₁₋₄-alkyl, unless otherwise noted, is also meant to include those derivatives of alkyl defined in more detail below as “unsaturated alkyl”. An unsaturated alkyl group is one having one or more double bonds or triple bonds. Preferred unsaturated alkyl substituents are vinyl, 2-propenyl or prop-2-yn-1-yl.

In the context of the present invention the term “C₁₋₄-alkyl having one or more hydrogen atoms in alkyl optionally replaced by deuterium” encompasses, but is not limited to the following residues: —CD₃, —CH₂D, —CHD₂, CD₃CH₂(CH₂)ₙ—, CD₃CH₂(CHD)ₙ-, CD₃CH₂(CD₂)ₙ-, CH₂DCH₂(CH₂)ₙ—, CH₂DCH₂(CHD)ₙ-, CH₂DCH₂(CD₂)ₙ-, CHD₂CH₂(CH₂)ₙ—, CHD₂CH₂(CHD)ₙ-, CHD₂CH₂(CD₂)ₙ-, CD₃CHD(CH₂)ₙ—, CD₃CHD(CHD)ₙ-, CD₃CHD(CD₂)ₙ-, CH₂DCHD(CH₂)ₙ—, CH₂DCHD(CHD)ₙ-, CH₂DCHD(CD₂)ₙ-, CHD₂CHD(CH₂)ₙ—, CHD₂CHD(CHD)ₙ-, CHD₂CHD(CD₂)ₙ-, CH₃CHD(CH₂)ₙ—, CH₃CHD(CHD)ₙ-, CH₃CHD(CD₂)ₙ-, CD₃CD₂(CH₂)ₙ—, CD₃CD₂(CHD)ₙ-, CD₃CD₂(CD₂)ₙ-, CH₂DCD₂(CH₂)ₙ—, CH₂DCD₂(CHD)ₙ-, CH₂DCD₂(CD₂)ₙ-, CHD₂CD₂(CH₂)ₙ—, CHD₂CD₂(CHD)ₙ-, CHD₂CD₂(CD₂)ₙ-, CH₃CD₂(CH₂)ₙ—, CH₃CD₂(CHD)ₙ-, CH₃CD₂(CD₂)ₙ-, wherein n is an integer from 0 to 2, and CH₃CH₂(CHD)ₘ—, CH₃CH₂(CD₂)ₘ—, wherein m is an integer from 1 to 2, as well as —CD(CD₃)₂, —CH(CD₃)₂ and —C(CD₃)₃. Preferred C₁₋₂-alkyl containing deuterium are —CD₃ and —CD₃CD₂, most preferred is —CD₃.

A “C₀₋₆-alkylene” means that the respective group is divalent and connects the attached residue with the remaining part of the molecule. Moreover, in the context of the present invention, “C₀-alkylene” is meant to represent a bond, whereas C₁-alkylene means a methylene linker, C₂-alkylene means a ethylene linker or a methyl-substituted methylene linker and so on. In the context of the present invention, a C₀₋₆-alkylene preferably represents a bond, a methylene, a ethylene group or a propylene group. The term “alkylene”, unless otherwise noted, is also meant to include a unsaturated divalent chain, if appropriate (i.e. possible for “C₂₋₆-alkylene”). A representative example for an unsaturated C₄-alkylene is —CH₂—CH═CH—CH₂—.

The term “fluoro-C₁₋₄-alkyl” or “O-fluoro-C₁₋₄-alkyl”, respectively, means that one or more hydrogen atoms in the alkyl chain are replaced by one or more fluoro atoms. Preferred are CHF₂, CF₃, CH₂CF₃ and CF₂CF₃. A more preferred example thereof is the formation of a —CF₃ group.

Similar applies to “halo-C₁₋₄-alkyl” or “O-halo-C₁₋₄-alkyl”, which means that one or more hydrogen atoms in the alkyl chain are replaced by one or more halogen atoms, independently selected from fluoro, chloro, bromo and iodo.

In the context of the present invention the term “fluoro-C₁₋₄-alkyl having one or more hydrogen atoms in alkyl optionally replaced by deuterium” means, that if the fluoro-C₁₋₄-alkyl contains one or more hydrogen atom(s), one or more hydrogen(s) can be replaced by fluorine(s), yielding the same as described above for the term “C₁₋₄-alkyl having one or more hydrogen atoms in alkyl optionally replaced by deuterium”. It is understood, that fluoro-C₁₋₄-alkyl can also be completely fluorinated. Preferred are fluoro-C₁₋₂-alkyl containing deuterium such as CDF₂, CD₂CF₃ and CD₂CF₂D. Most preferred is CDF₂. A “3- to 10-membered cycloalkyl” group means a saturated or partially unsaturated mono-, bi-, spiro- or multicyclic ring system comprising 3 to 10 carbon atoms, wherein each of the atoms forming the ring system (i.e. skeletal atoms) is a carbon atom. Examples include cyclopropyl, cyclobutyl, cyclopentyl, cyclohexyl, cyclohexenyl, bicyclo[2.2.2]octyl, bicyclo[3.2.1]octanyl, spiro[3.3]heptyl, bicyclo[2.2.1]heptyl, adamantyl and pentacyclo[4.2.0.0^{2,5}.0^{3,8}.0^{4,7}]octyl. Consequently, a 3- to 6-membered cycloalkyl group means a saturated or partially unsaturated mono- bi-, or spirocyclic ring system comprising 3 to 6 carbon atoms whereas a 5- to 8-membered cycloalkyl group means a saturated or partially unsaturated mono-, bi-, or spirocyclic ring system comprising 5 to 8 carbon atoms.

The term “3- to 6-membered cycloalkyl” encompasses, but is not limited to cyclopropyl, cyclobutyl, cyclopentyl, cyclohexyl, bicyclo[1.1.1]pentyl, bicyclo[2.1.0]pentyl and spiro[2.3]hexanyl. More preferred is cyclopropyl or cyclobutyl.

A “3- to 10-membered heterocycloalkyl containing 1 to 4 heteroatoms independently selected from N, O and S” group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O or S. The sulfur heteroatom in the ring can also be oxidized to S═O or SO₂. The carbon atom in the ring can also be oxidized to C═O. Examples thereof include epoxidyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom. An example for a S-linked heterocycloalkyl is the cyclic sulfonimidamide

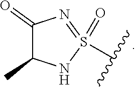 [SMILES: *S1(=O)=NC(=O)[C@H](C)N1]

The term “3- to 6-membered heterocycloalkyl” encompasses, but is not limited to epoxidyl, oxetanyl, pyrrolidinyl, tetrahydrofuranyl, piperidinyl, piperazinyl, 2-oxaspiro[3.3]heptyl, tetrahydropyranyl, 1,4-dioxanyl, morpholinyl and the like.

A “6- or 10-membered aryl” is phenyl or naphthyl.

A “5- to 10-membered heteroaryl containing 1 to 6 heteroatoms independently selected from N, O and S” means a 5- to 10-membered mono- or bicyclic heteroaromatic ring system (within the application also referred to as heteroaryl) containing up to 6 heteroatoms independently selected from N, O and S. Examples of monocyclic heteroaromatic rings include pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzofuranyl, benzoxazolyl, indolyl, indolizinyl 1,5-naphthyridinyl, 1,7-naphthyridinyl and pyrazolo[1,5-a]pyrimidinyl. The nitrogen or sulphur atom of the heteroaryl system may also be optionally oxidized to the corresponding N-oxide, S-oxide or S,S-dioxide. “5-membered heteroaryl” means a monocyclic aromatic ring system containing up to 3 heteroatoms independently selected from N, O and S. Examples of monocyclic heteroaromatic rings include pyrrolyl, imidazolyl, furanyl, thiophenyl and oxazolyl. The sulfur heteroatom in the ring can also be oxidized to S═O or SO₂.

A 5-membered heterocyclopentenyl group means a partially unsaturated 5-membered carbon monocyclic ring wherein 1 or 2 carbon atoms are replaced by 1 or 2 heteroatoms, respectively, wherein the heteroatoms are independently selected from N, O and S. Examples thereof include 2,3-dihydrofuranyl, 2,5-dihydrofuranyl, 2,5-dihydrothiophenyl or 2,5-dihydro-1H-pyrrole. The sulfur heteroatom in the ring can also be oxidized to S═O or SO₂.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

The term “diastereomer” means stereoisomers that are not mirror images of one another and are non-superimposable on one another. The term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Further the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, hydrate, or solvate thereof, in a particular crystal packing arrangement. All polymorphs have the same elemental composition. The term “crystalline” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility.

The term “effective amount” is meant to include the amount of a compound that, when administered, is sufficient to prevent development of, or alleviate to some extent, one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g. a rodent, a mouse, a rat, a rabbit, a monkey, a dog, a cat, a sheep, cattle, a primate or a pig). In some embodiments, subjects include, but are not limited to, mammals, birds, reptiles, amphibians, fish or worms. In some embodiments, a subject may be a transgenic animal, genetically-engineered animal or a clone.

It has unexpectedly been found that compounds as detailed herein show beneficial effects, e.g. higher microsomal stability. The following example section shows further details.

With the above context, the following consecutively numbered embodiments provide further specific aspects of the invention:

1. A compound of Formula (I):

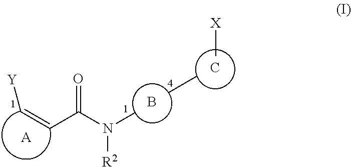

or an enantiomer, diastereomer, tautomer, solvate, or pharmaceutically acceptable salt thereof, wherein
A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, —CN, —NO₂, oxo, —OH, C₁₋₄-alkyl, —O—C₁₋₄-alkyl, fluoro-C₁₋₄-alkyl and —O-fluoro-C₁₋₄-alkyl, ring A having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²³)ₘR²¹, C₀₋₆-alkylene-NR²¹S(═O)ₓ(═NR²³)_{y}R²¹, C₀₋₆-alkylene-S(═O)ₓ(═NR²³)_{y}NR²¹R²², C₀₋₆-alkylene-NR²¹S(═O)ₓ(═NR²³)_{y}NR²¹R²², C₀₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹, C₀₋₆-alkylene-NR²¹R²²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl; and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl, and wherein the residue —NR² on ring B is in a 1,4-orientation with respect to ring C,
B having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆ alkylene-S(═O)ₓ(═NR³³)_{y}NR³¹R³², C₀₋₆-alkylene-NR³¹S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
C having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X having one or more hydrogen atoms optionally replaced by deuterium;
Y is selected from —CONH—CN, —CONHOH, —CONHOR¹⁰, —CONR¹⁰OH, —C(═NOH)NR¹¹R¹², —CONHS(═O)ₓ(═NR¹³)_{y}R¹⁰, —CONHS(═O)_{y}(═NR¹³)_{y}NR¹¹R¹², —SO₃H, —S(═O)ₓ(═NR¹³)_{y}NHCOR¹⁰, —S(═O)ₓ(═NR¹³)_{y}NHR¹¹, —P(═O)(OH)₂, —P(═O)(NR¹¹R¹²)OH, —P(═O)R¹¹(OH), —B(OH)₂,

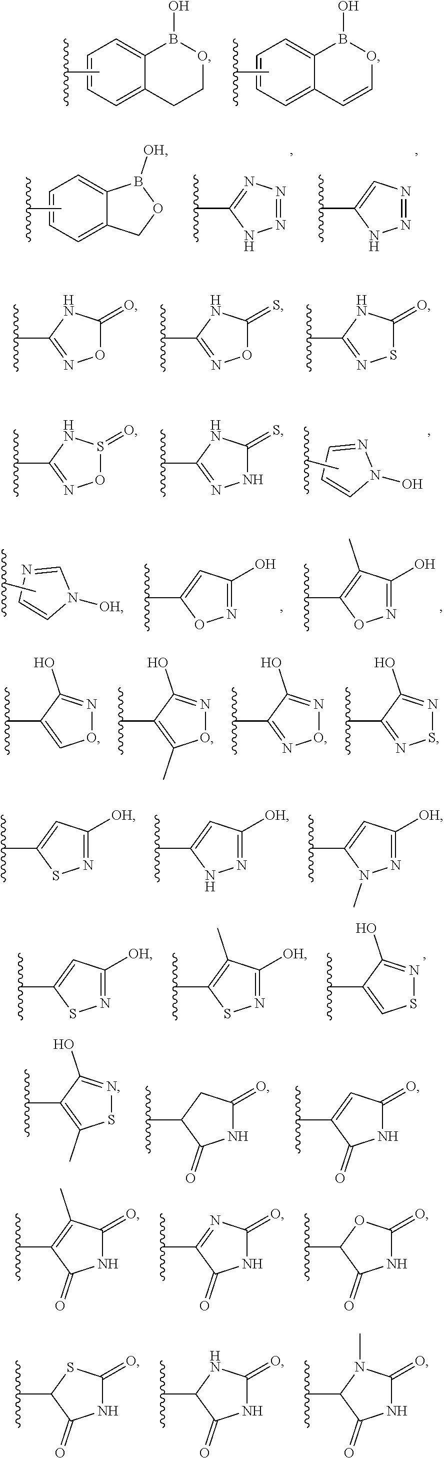

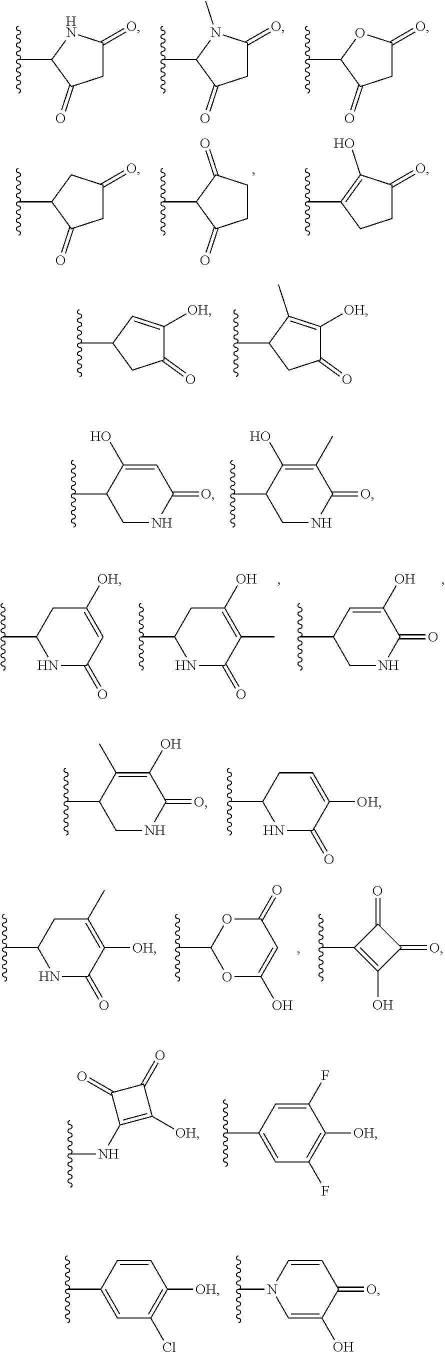

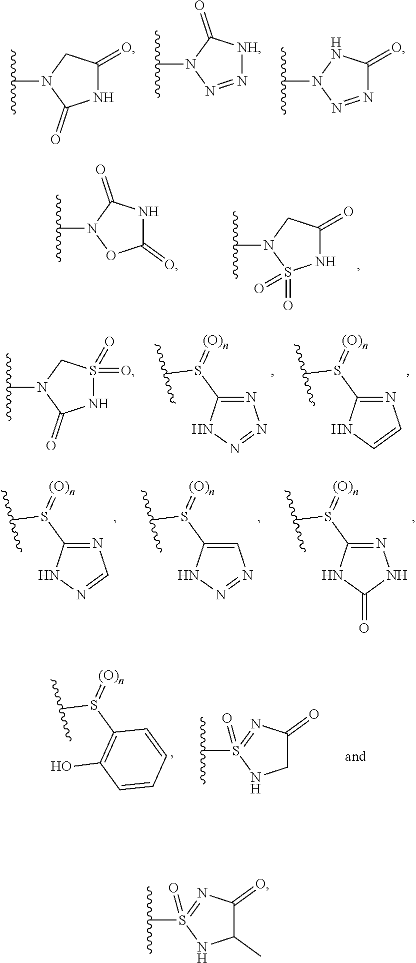

Y having one or more hydrogen atoms optionally replaced by deuterium;
R² is selected from H and C₁₋₆-alkyl,
wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² having one or more hydrogen atoms optionally replaced by deuterium;
R¹⁰ is selected from C₁₋₆-alkyl, 3- to 6-membered cycloalkyl or 3- to 6-membered heterocycloalkyl,
wherein alkyl, cycloalkyl and heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹⁰ having one or more hydrogen atoms optionally replaced by deuterium;
R¹¹, R¹², R²¹, R²², R³¹, R³², R⁴¹, R⁴² are independently selected from H, C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹¹ and/or R¹² and/or R²¹ and/or R²² and/or R³¹ and/or R³² and/or R⁴¹ and/or R⁴² having one or more hydrogen atoms optionally replaced by deuterium;
or R¹¹ and R¹², R²¹ and R²², R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R¹¹ and/or R¹² and/or R²¹ and/or R²² and/or R³¹ and/or R³² and/or R⁴¹ and/or R⁴² having one or more hydrogen atoms optionally replaced by deuterium;
R¹³, R²³, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹³ and/or R²³ and/or R³³ and/or R⁴³ having one or more hydrogen atoms optionally replaced by deuterium;
n, m, x, y are independently selected from 0 to 2;
with the proviso that the sum of integer m and n for the residue linked to the same sulfur atom is independently selected from 0 to 2;
with the proviso that the sum of integer x and y for the residue linked to the same sulfur atom is independently selected from 1 or 2;
and with the proviso, that the following structure is excluded:

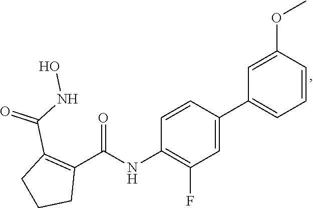 [SMILES: COc1cccc(-c2ccc(NC(=O)C3=C(C(=O)NO)CCC3)c(F)c2)c1]

2. A compound of Formula (I) according to embodiment 1, or a solvate or pharmaceutically acceptable salt thereof, wherein

Y is selected from —CONH—CN, —CONHOR¹⁰, —CONR¹⁰OH, —C(═NOH)NR¹¹R¹², —CONHS(═O)ₓ(═NR³)_{y}R¹⁰, —CONHS(═O)_{y}(═NR¹³)_{y}NR¹¹R¹²,

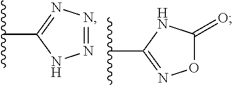

R¹⁰ is selected from C₁₋₃-alkyl, cyclopropyl or oxetan-3-yl,
wherein alkyl, cyclopropyl or oxetan-3-yl is unsubstituted or substituted with 1 to 3 substituents independently selected from F, —CN, Me, CHF₂, CF₃, —OH, oxo, —OMe, —OCHF₂ and —OCF₃, R¹⁰ having one or more hydrogen atoms optionally replaced by deuterium;
R¹¹ and R¹² are independently selected from H or C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹¹ and/or R¹² having one or more hydrogen atoms optionally replaced by deuterium;
R¹³ is selected from H, —CN and C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹³ having one or more hydrogen atoms optionally replaced by deuterium;
x is 1 and y is 1 or x is 2 and y is 0.

3. A compound of Formula (I) according to embodiment 1 or 2, or a solvate or pharmaceutically acceptable salt thereof, wherein

Y is selected from —CONH—CN, —CONHOR¹⁰, —C(═NOH)NR¹¹R¹², —CONHS(═O)ₓ(═NR¹³)_{y}R¹⁰, —CONHS(═O)_{y}(═NR¹³)NR¹¹R¹²,

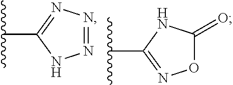

R¹⁰ is selected from C₁₋₃-alkyl, cyclopropyl or oxetan-3-yl,
wherein alkyl, cyclopropyl or oxetan-3-yl is unsubstituted or substituted with 1 to 3 substituents independently selected from F, —CN, Me, CHF₂, CF₃, —OH, oxo, —OMe, —OCHF₂ and —OCF₃, R¹⁰ having one or more hydrogen atoms optionally replaced by deuterium;
R¹¹ and R¹² are independently selected from H or C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹¹ and/or R¹² having one or more hydrogen atoms optionally replaced by deuterium;
R¹³ is selected from H, —CN and C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹³ having one or more hydrogen atoms optionally replaced by deuterium;
x is 1 and y is 1 or x is 2 and y is 0.

4. A compound of Formula (I) according to any of embodiments 1 to 3, wherein

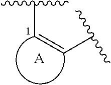

is selected from

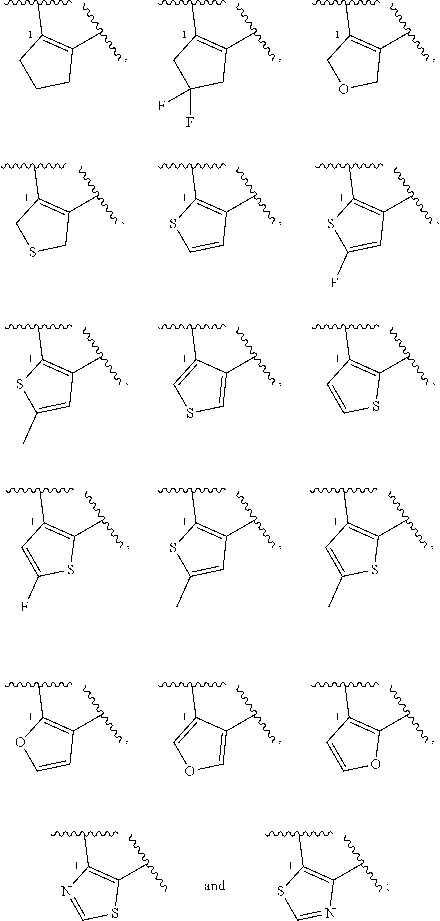

### and R² is H.

5. A compound of Formula (I) according to any of embodiments 1 to 4, wherein

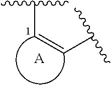

is selected from

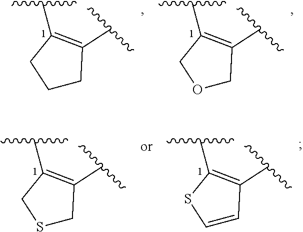 [SMILES: *C1=C(*)CCC1 ; *C1=C(*)COC1 ; *C1=C(*)CSC1 ; *c1ccsc1*]

### and R² is H.

6. A compound of Formula (I) according to any of embodiments 1 to 5, wherein one or more hydrogen atom(s) in any substituent is replaced by deuterium.

7. A compound of Formula (I) according to any of embodiments 1 to 6, wherein

B is phenyl,
wherein phenyl is unsubstituted or substituted with 1 to 4 substituents independently selected from the group consisting of D, F, Cl, —CN, Me, CD₃, CHF₂ and CF₃;
and wherein the residue —NR² on ring B is in a 1,4-orientation with respect to ring C.

8. A compound of Formula (I) according to any of embodiments 1 to 7, wherein

C is phenyl,
wherein phenyl is unsubstituted or substituted with 1 to 4 substituents independently selected from the group consisting of D, F, Cl, —CN, Me, CD₃, CHF₂, CF₃, —OMe, —OCD₃, —OCHF₂ and —OCF₃;
X is selected from D, F, Cl, —CN, Me, CD₃, CHF₂, CF₃, Et, CD₂CD₃, —OMe, —OCD₃, —OCHF₂, —OCF₃, —OEt and —OCD₂CD₃.

9. A compound of Formula (I) according to any of claims 1 to 8, wherein

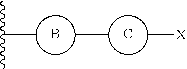

is selected from

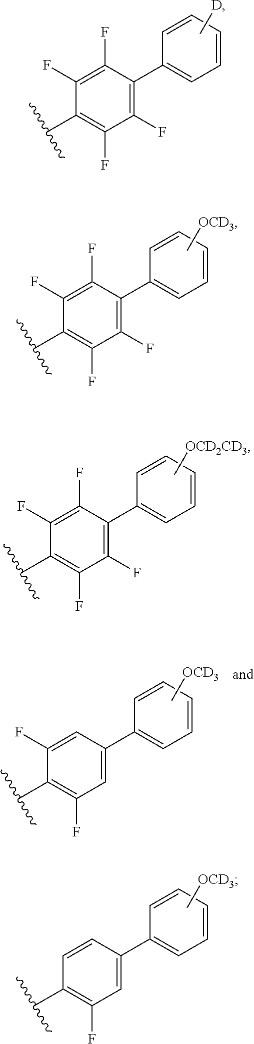

wherein ring C is optionally substituted with 1 to 4 substituents independently selected from D or F.

10. A compound of Formula (I) according to any of embodiments 1 to 8, wherein

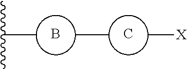

is selected from

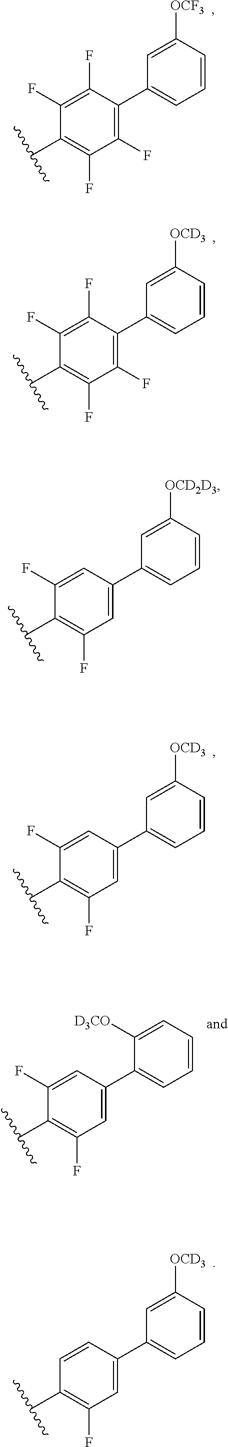

11. A compound of Formula (I) according to any of claims 1 to 10, wherein

Y is selected from

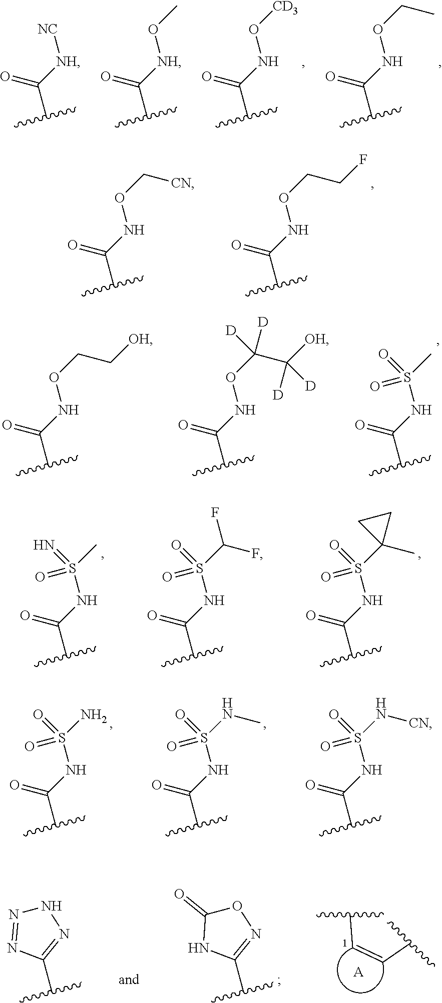

is selected from

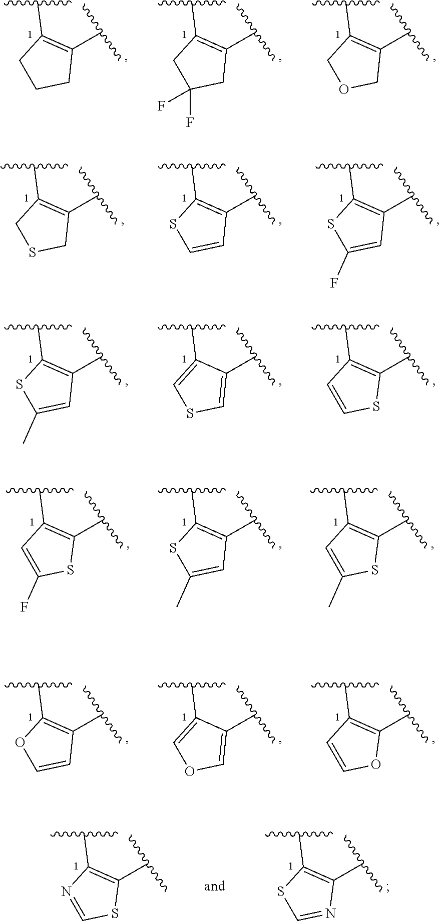

### R² is H;

B is selected from

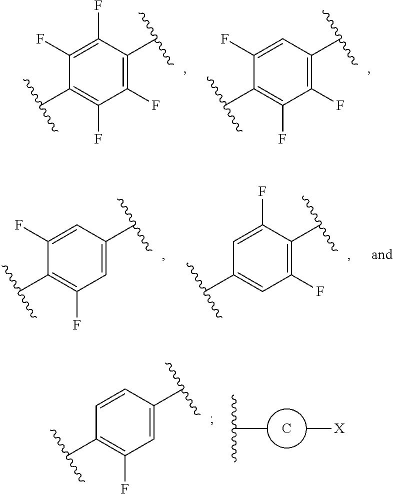

is selected from

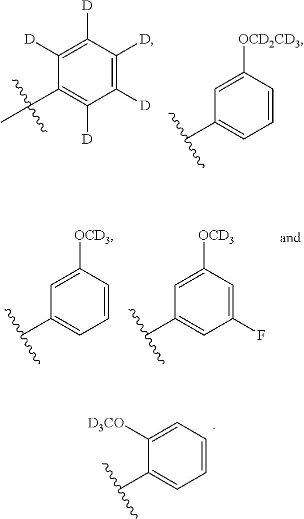

12. A compound of Formula (I) according to any of embodiments 1 to 10, wherein
Y is selected from

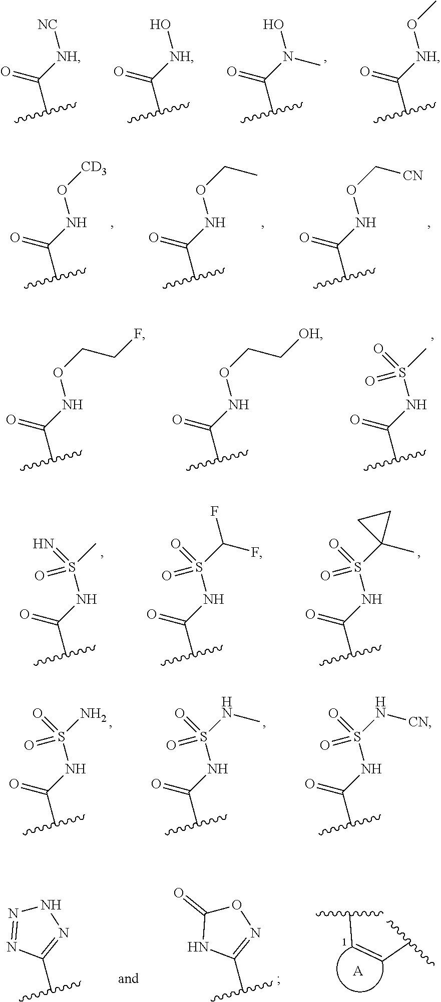

is selected from

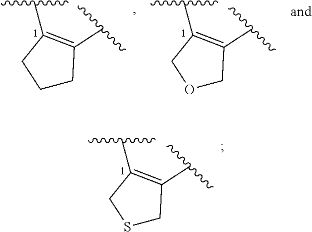 [SMILES: *C1=C(*)CCC1 ; *C1=C(*)COC1 ; *C1=C(*)CSC1]

### R² is H;

B is selected from

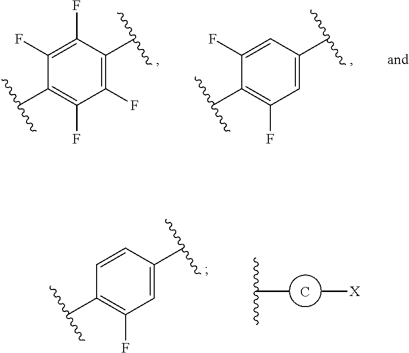

is selected from

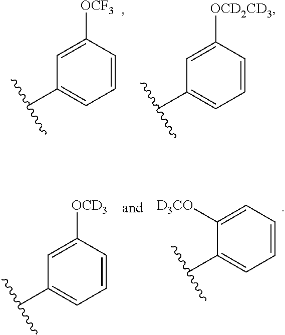

13. A compound of Formula (I) according to any of claims 1 to 12, which is selected from

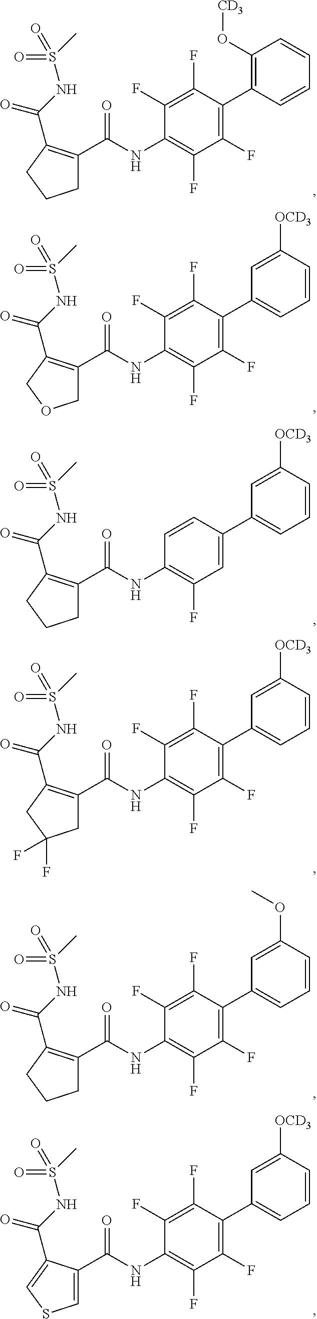

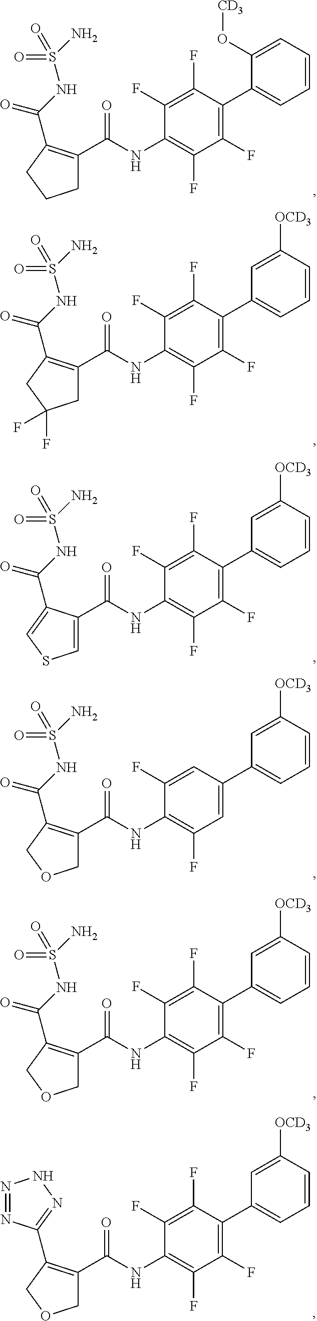

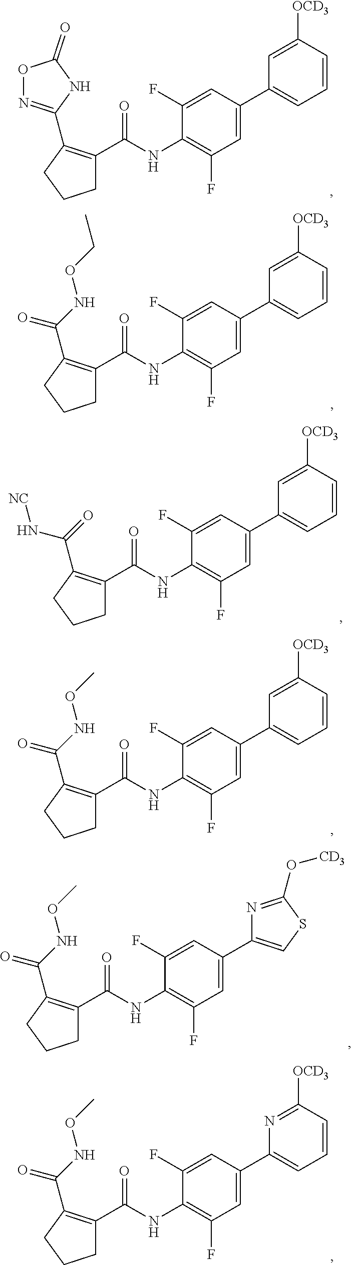

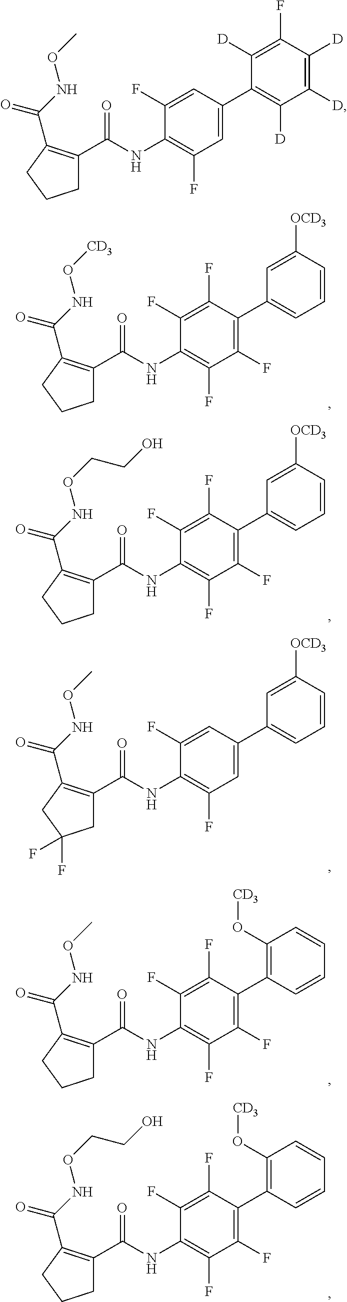

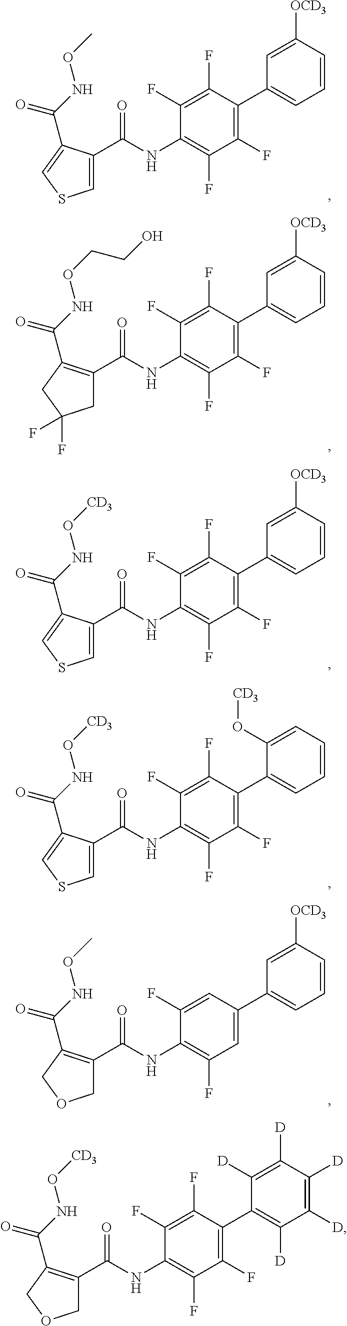

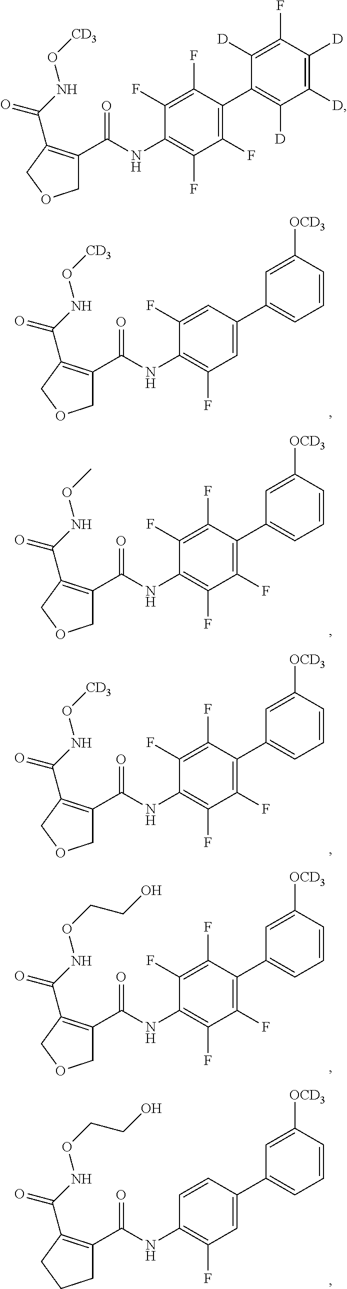

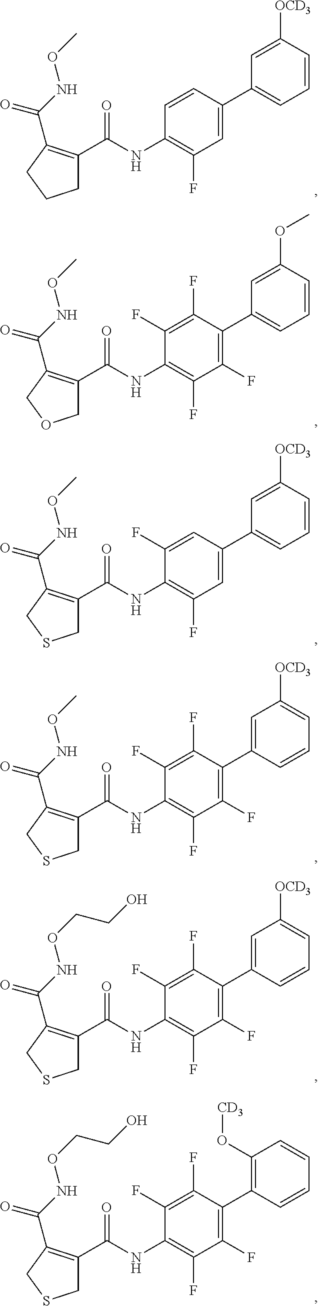

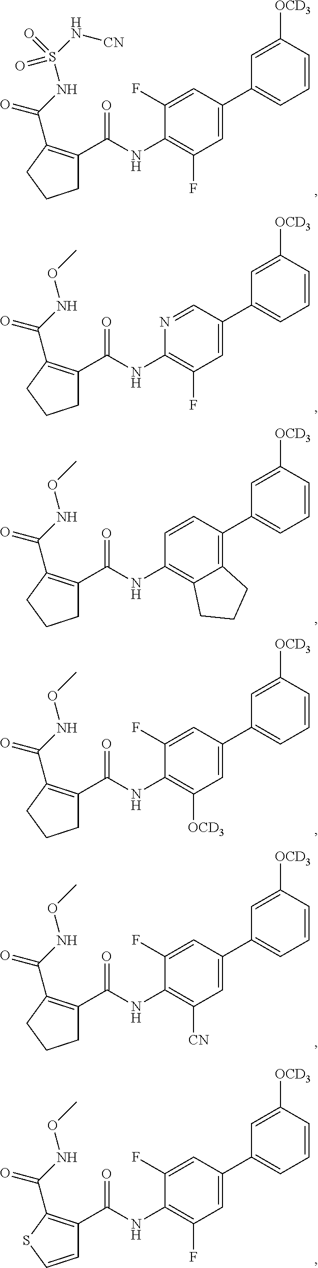

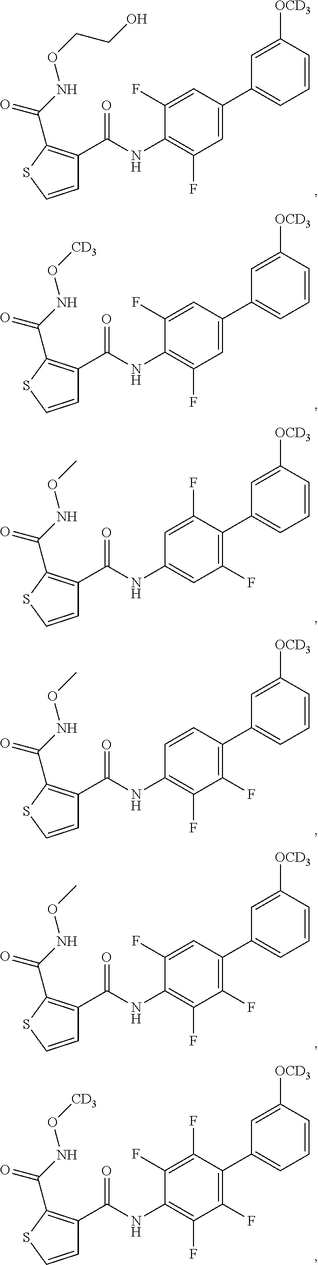

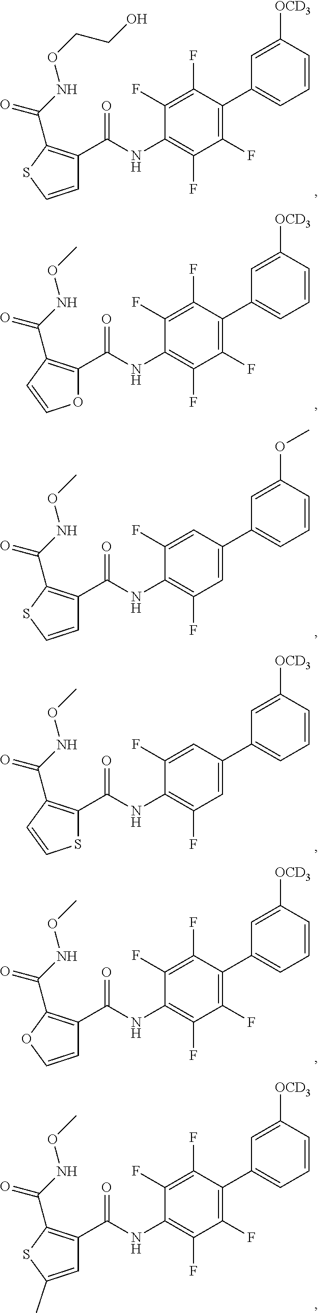

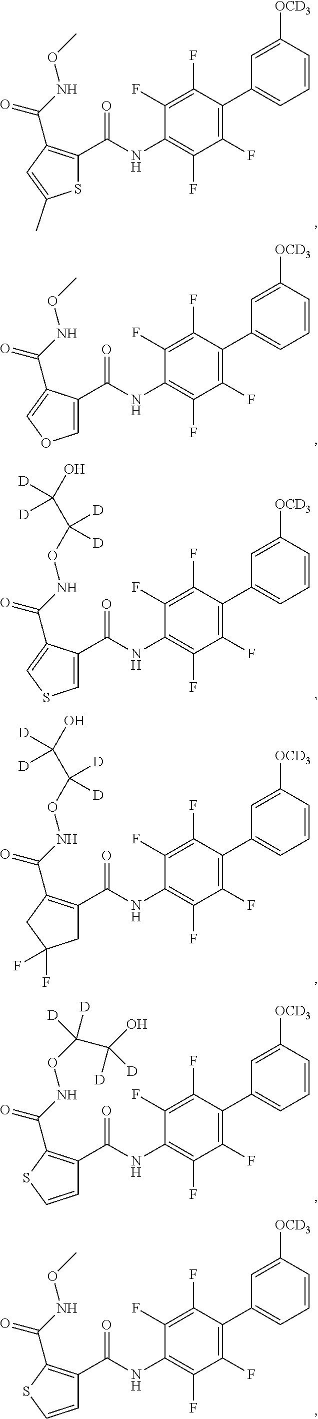

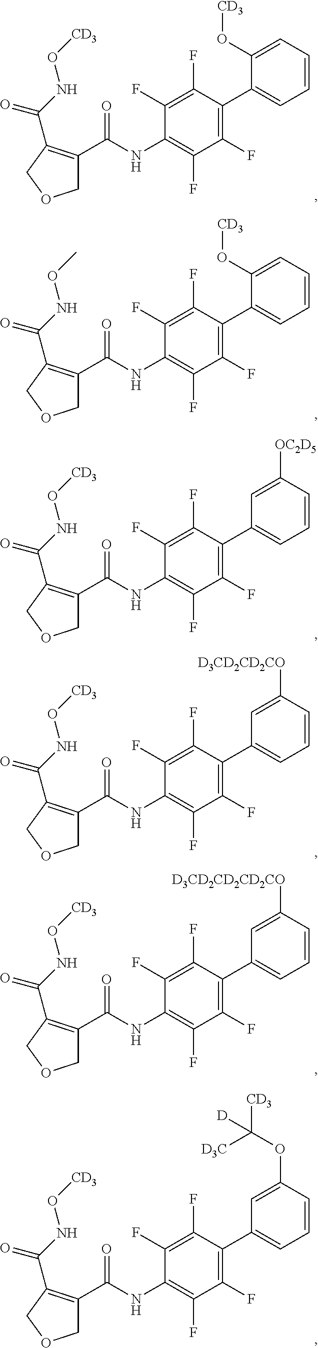

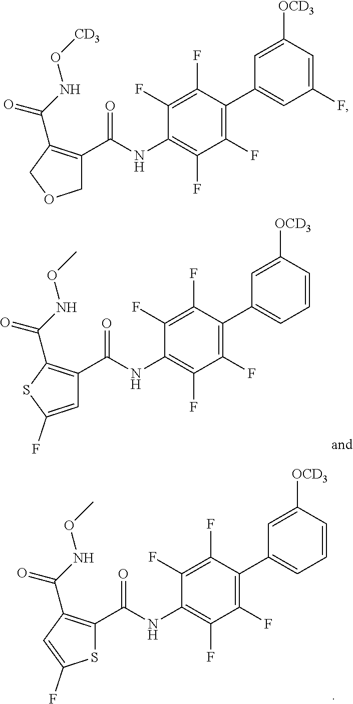

or a solvate or pharmaceutically acceptable salt thereof.

14. A compound of Formula (I) according to any of embodiments 1 to 13, which is selected from

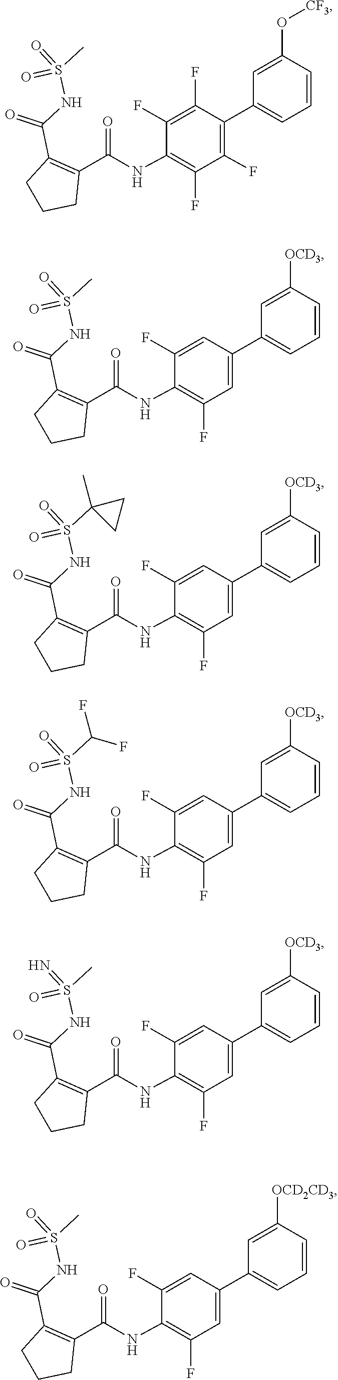

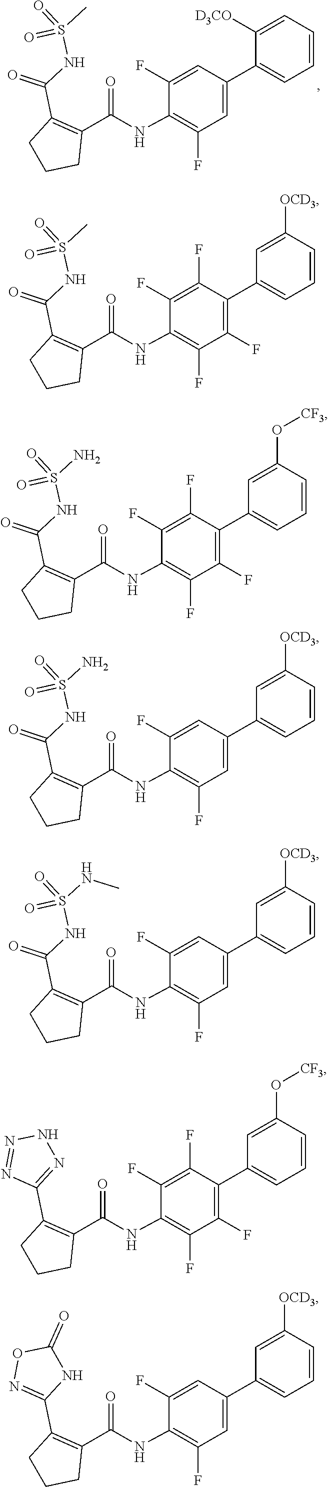

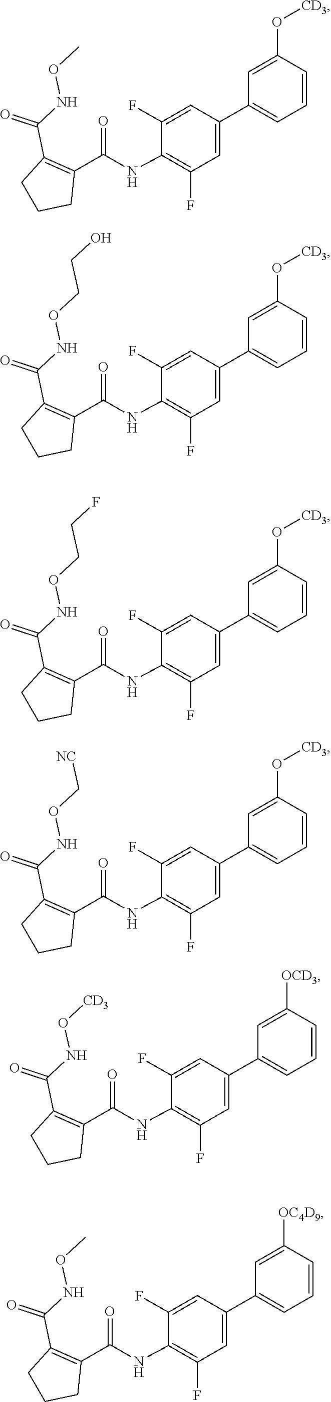

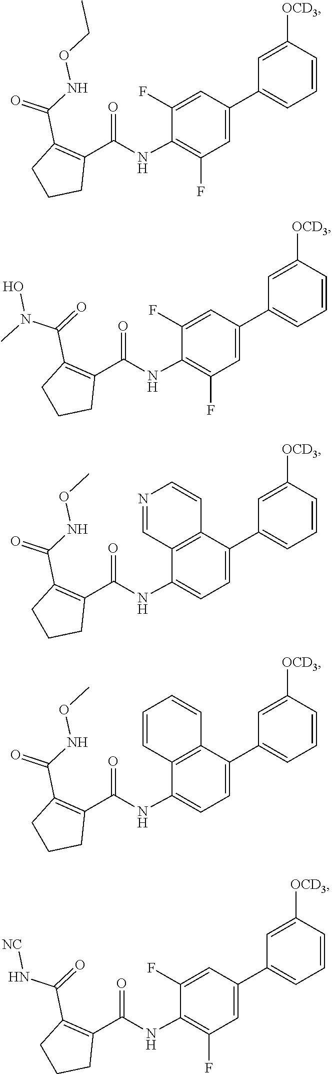

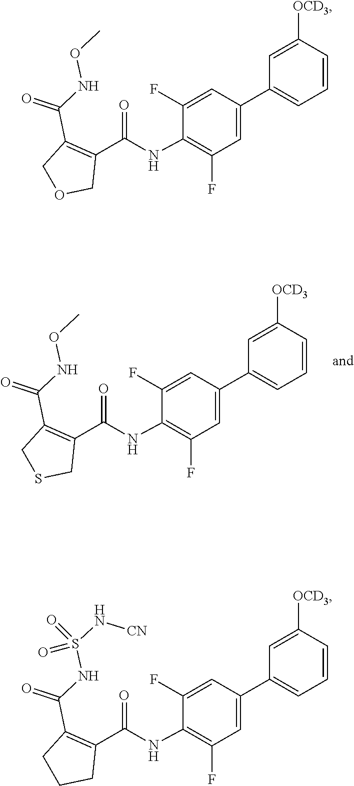

or a solvate or pharmaceutically acceptable salt thereof.

15. A compound according to any one of the preceding embodiments for the use as a medicament.

16. A compound according to any one of embodiments 1 to 15 for use in the prophylaxis and/or treatment of diseases, disorders, therapeutic indications or medical conditions amenable for treatment with DHODH inhibitors.

17. A compound for use according to embodiment 16 wherein the disease, disorder, therapeutic indication or medical condition is selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.

18. A compound for use according to embodiment 17 wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

19. A pharmaceutical composition comprising a compound according to any one of embodiments 1 to 14 and a pharmaceutically acceptable carrier or excipient.

20. A pharmaceutical composition of embodiment 19, further comprising one or more additional therapeutic agents selected from anti-inflammatory agents, anti-viral agents, immunosuppressive and/or immunomodulatory agents, steroids, non-steroidal anti-inflammatory agents, antihistamines, analgesics and suitable mixtures thereof.

### EXPERIMENTAL PART

The carboxylic acid containing intermediates of the present invention can be prepared as outlined in WO2003/006425 and WO2004/056797 (and references cited therein). By using appropriate deuterated building blocks or via hydrogen-deuterium exchange (e.g. Synthesis 2019; 51:1319 or Angew. Chem. Int. Ed. 2018; 57:3022) the deuterated intermediates can be prepared.

The compounds of the present invention can be prepared by a combination of methods known in the art including the procedures described in Schemes I below. The synthetic route starts with the Suzuki coupling (V=boronic acid or boronic ester, W=Br, I or OMs; or opposite functionalization) of B-ring I-a with C-ring I-b or by using another C—C-coupling procedure (e.g. Example 4). The amino group of I-c is the reacted with carboxylic acid I-d (H═H or alkyl, e.g. Example 16 or Example 9, respectively) or anhydride I-e (e.g. Example 5) via an amide coupling (and optionally saponification of the ester in case of R=alkyl to furnish carboxylic acid I-f. It may be necessary to separate the two regioisomers have been formed (or after functionalization towards new residue Y). Finally the carboxylic acid is transformed to residue Y in Formula (I), e.g. by coupling a alkoxyamine (e.g. Example 4), alkylsulfonamide (e.g. Example 1) or optionally substituted sulfuric diamide (e.g. Example 2) or manipulation towards a tetrazole (e.g. Example 3) or oxadiazole (e.g. Example 4). Compounds of Formula (I) can also directly prepared by amide coupling for suitable functionalized A-ring carboxylic acid I-g with amine I-c (e.g. Example 10).

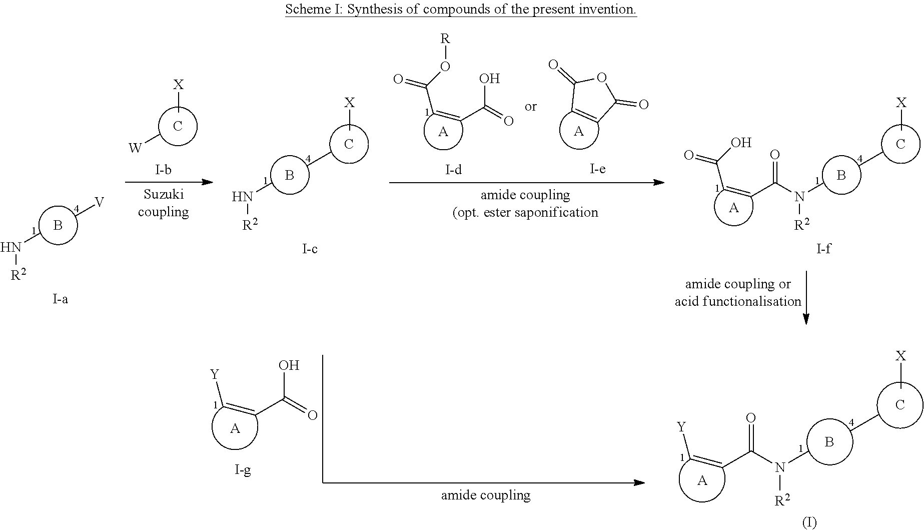

### Abbreviations

Ac acetyl
aq. aqueous
Boc tert-butyloxycarbonyl
dba dibenzylideneacetone
DCM dichloromethane
DIPEA N,N-diisopropylethylamine
DMF N,N-dimethylformamide
DMSO dimethyl sulfoxide
dppf 1,1′-bis(diphenylphosphino)ferrocene
EA ethyl acetate
EDCI 1-ethyl-3-(3-dimethylaminopropyl)carbodiimide
FCC flash chromatography on silica gel
PE petroleum ether
Ph phenyl
prep. preparative
rt room temperature (20±4° C.)
TCFH chloro-N,N,N′,N′-tetramethylformamidinium hexafluorophosphate
TEA triethylamine
Tf triflate

### Experimental Section

### Preparative Example P1

### Step 1: tert-Butyl (methoxy-d3)carbamate (P1a)

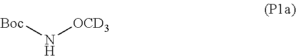

To a solution of tert-butyl hydroxycarbamate (10 g) in MeCN (20 mL) was added K₂CO₃ (31 g) and CD₃I (4.7 mL). The mixture was stirred at 65° C. for overnight, colled, filtered, concentrated and purified by FCC (PE:EA=20:1) to give compound P1a as a colorless oil. ¹H-NMR (400 MHz, DMSO-d6) δ 7.23 (s, 1H), 1.49 (s, 9H). LCMS (ESI): m/z 173.1 (M+Na)⁺.

### Step 2: O-(Methyl-d3)hydroxylamine hydrochloride (P1)

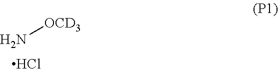

To a solution of compound P1a (7.0 g) in 1,4 dioxane (15 mL) was added 4M HCl in dioxane (15 mL) and the mixture was stirred at rt for 16 h. The mixture was filtered and the filter cake was washed with 1,4 dioxane (15 mL) and then with PE twice again. The solid was dried in vacuum to afford P1 as a white solid. ¹H-NMR (400 MHz, DMSO-d6) δ 11.03 (s, 3H). LCMS (ESI): m/z 51.1 (M−Cl)⁺.

### Preparative Example P1/1

### Step 1: 2-(2-Hydroxyethoxy-1,1,2,2-d4)isoindoline-1,3-dione (P1/1a)

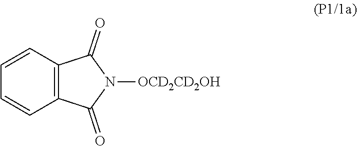

By reacting 2-hydroxyisoindoline-1,3-dione with 2-bromoethan-1,1,2,2-d4-1-ol in MeCN and NEt₃ similar as described for the undeuterated bromide in WO2014/081025 the intermediate P1/1a can be prepared.

### Step 2: 2-(Aminooxy)ethan-1,1,2,2-d4-1-ol (P1/1)

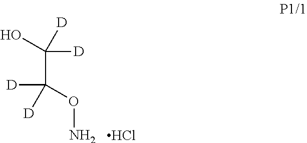

By reacting intermediate P1/1a with hydrazine in ethanol similar as described for the undeuterated alcohol in J. Chem. Soc. Perkin Trans. 1 1987:2829, then slurrying the free amine in 4M HCl in 1,4-dioxane and finally evaporating the solvent the building block P1/1 can be prepared.

### Preparative Example P2

### 4-Bromo-2-fluoro-6-(methoxy-d3)aniline (P2)

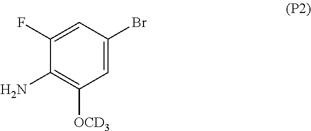

To a solution of 2-amino-5-bromo-3-fluorophenol (300 mg) in MeCN (5 mL) was added K₂CO₃ (0.4 g) and CD₃I (0.15 mL). The mixture was stirred at 65° C. for overnight, cooled to rt, filtered concentrated and purified by FCC (PE:EA=20:1) to give compound P2 as an oil. LCMS (ESI): m/z 223.2/225.1 (M+H)⁺.

### Preparative Example P3

### Step 1: bis(3-Methoxyphenyl)zinc (P3a)

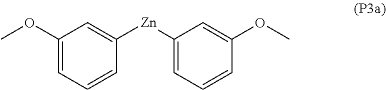

To the mixture of (3-methoxyphenyl)magnesium bromide (42 mL, 1M in THF) was added LiCl (2.67 g) and ZnCl₂ (20 mL, 1M in THF) at rt. The mixture was stirred at rt for 1 h to give compound P3a as a solution in THF.

### Step 2: 1-(1,3-Dioxoisoindolin-2-yl) 4-methyl bicyclo[2.2.2]octane-1,4-dicarboxylate (P3b)

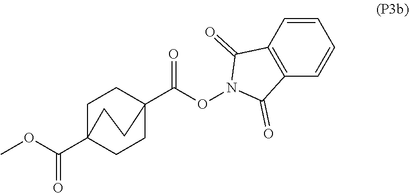 [SMILES: COC(=O)C12CCC(C(=O)ON3C(=O)c4ccccc4C3=O)(CC1)CC2]

N,N-Diisopropylcarbodiimide (3.6 g) was added to a solution 4-(methoxycarbonyl)bi-cyclo[2.2.2]octane-1-carboxylic acid (5.0 g), 2-hydroxyisoindoline-1,3-dione (3.8 g), DMAP (864 mg) in CH₂Cl₂ (50 mL) at rt under a nitrogen atmosphere. The mixture was stirred at rt overnight, washed with H₂O (2×300 mL), dried (Na₂SO₄), filtered, concentrated and purified by FCC to give compound P3b as a white solid. LCMS (ESI): m/z 380.2 (M+Na)⁺.

### Step 3: Methyl 4-(3-methoxyphenyl)bicyclo[2.2.2]octane-1-carboxylate (P3c)

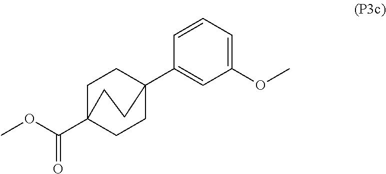

Compound P3a (˜20 mmol, as THF solution) was added to a solution of compound P3b (3.0 g), 2-methyl-6-(6-methyl-2-pyridyl)pyridine (0.93 g), Nickel(II)-acetylacetonat (1.08 g) and CH₃CN (50 mL) at room temperature. The mixture was degassed with 3 vacuum-nitrogen cycles, stirred at 80° C. overnight, cooled to rt and concentrated. The residue was diluted with water (50 mL) and extracted with EtOAc (3×50 mL). The combined organic layer was washed with brine (2×50 mL), dried (Na₂SO₄), filtered, concentrated and purified by FCC to give compound P3c as a light yellow solid. LCMS (ESI): m/z 275.3 (M+H)⁺.

### Step 4: Methyl 4-(3-hydroxyphenyl)bicyclo[2.2.2]octane-1-carboxylate (P3d)

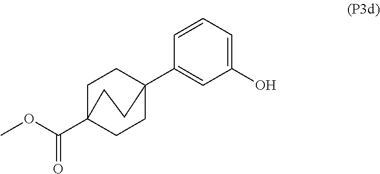 [SMILES: COC(=O)C12CCC(c3cccc(O)c3)(CC1)CC2]

To a mixture of compound P3c (1.9 g) in DCM (40 mL) was added BBr₃ (1M, 10 mL) and the mixture was stirred at rt for 4 h, poured into water (50 mL) and extracted with DCM (3×30 mL). The combined organic layer was washed with brine, dried, filtered, concentrated and purified by FCC to give compound P3d as a white solid. LCMS (ESI): m/z 260.8 (M+H)⁺.

### Step 5: Methyl 4-(3-(methoxy-d3)phenyl)bicyclo[2.2.2]octane-1-carboxylate (P3e)

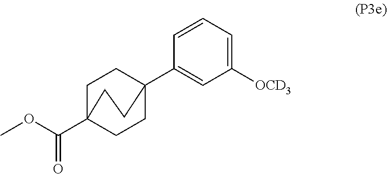

To the mixture of compound P3d (1.6 g) in CH₃CN (20 mL) was added CD₃I (1.8 g) and K₂CO₃ (1.7 g) and the mixture was stirred at 60° C. for 12 h, cooled to rt, poured into water (80 mL) and extracted with EA (3×30 mL). The combined organic layer was washed with brine, dried, filtered, concentrated and purified by FCC to give compound P3e as a white solid. LCMS (ESI): m/z 278.3 (M+H)⁺.

### Step 6: 4-(3-(Methoxy-d3)phenyl)bicyclo[2.2.2]octane-1-carboxylic acid (P3f)

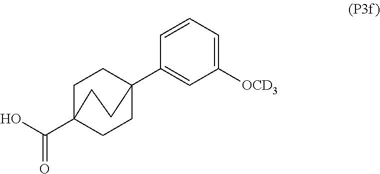

To a mixture of compound P3e (1.6 g) in MeOH (20 mL) was added LiOH (5 mL, 2M) and the mixture was stirred at rt for 12 h, concentrated and adjusted to pH=6 with 1N HCl. Then the mixture was purified by preparative HPLC to get compound P3f as a white solid. LCMS (ESI): m/z 264.0 (M+H)⁺.

### Step 7: tert-Butyl (4-(3-(methoxy-d3)phenyl)bicyclo[2.2.2]octan-1-yl)carbamate (P3f)

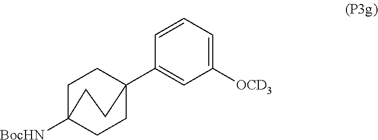

To a mixture of compound P3f (1.5 g) in toluene (30 mL) was added (Boc)₂O (1.3 g), diphenyl-phosphoryl azide (1.65 g) and TEA (1.2 g). The mixture was stirred at 80° C. for 12 h, cooled to rt, poured into water (80 mL) and extracted with EA (3×30 mL). The combined organic layer was washed with brine, dried, filtered, concentrated and purified by FCC to give compound P3g as a white solid. LCMS (ESI): m/z 335.0 (M+H)⁺.

### Step 7: 4-(3-(Methoxy-d3)phenyl)bicyclo[2.2.2]octan-1-amine hydrochloride (P3)

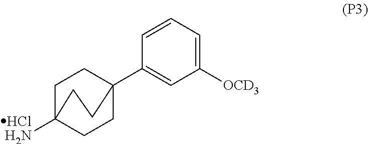

To a mixture of compound P3g (510 mg) in MeOH was added HCl (4M in MeOH). The mixture was stirred at rt for 3 h and concentrated to get compound P3 as a white solid. LCMS (ESI): m/z 235.3 (M−Cl)⁺.

### Preparative Example P4

### Step 1: 2-(3-(Methoxy-d3)phenyl)-4,4,5,5-tetramethyl-1,3,2-dioxaborolane (P4a)

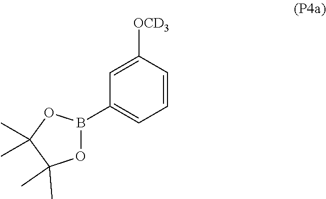

To a solution of 3-(4,4,5,5-tetramethyl-1,3,2-dioxaborolan-2-yl)phenol (10 g) in MeCN (50 mL) was added K₂CO₃ (18.8 g) and CD₃I (3.39 mL). The mixture was stirred at 65° C. overnight, cooled to rt, filtered, concentrated and purified by FCC (PE:EA=20:1) to give compound P4a as an oil.

### Step 2: 4-Bromo-2,3,6-trifluoroaniline (P4b)

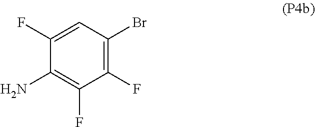 [SMILES: Nc1c(F)cc(Br)c(F)c1F]

To a solution of 2,3,6-trifluoroaniline (1 g) in DMF (20 mL) was added N-bromosuccinimide (1.2 g) and the mixture was stirred at 0° C. for 3 h, concentrated and purified by FCC (PE:EA=2:1) to give compound P4b as a yellow solid.

### Step 3: 2,3,5-Trifluoro-3′-(methoxy-d3)-[1,1′-biphenyl]-4-amine (P4)

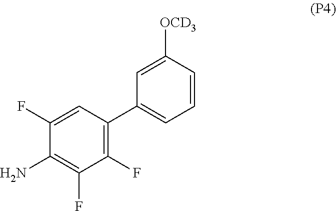

To a solution of compound P4a (300 mg) in 1,4-dioxane (20 mL) and H₂O (2 mL) was added compound P4b (379 mg), Cs₂CO₃ (1.3 g) and Pd(PPh₃)₄ (30 mg). The mixture was heated at 90° C. for 3 h, cooled, diluted with water and extracted with EtOAc (3×). The combined organic layer was separated, dried over Na₂SO₄, filtered, concentrated and purified by FCC (PE:EA=8:1) to give compound P4 as a yellow solid. LCMS (ESI): m/z 257.1 (M+H)⁺.

### Preparative Example P5

### 1H-Furo[3,4-c]pyrrole-1,3(5H)-dione (P5)

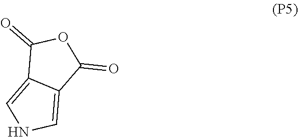

A solution of 1H-pyrrole-3,4-dicarboxylic acid (400 mg) in dry THF (50 mL) was added dicyclohexylcarbodiimide (797 mg). The mixture was stirred at 80° C. for 2 h, cooled to rt, concentrated and purified by prep. HPLC to afford compound P5 as a white solid.

### Preparative Example P6

### Step 1: 4,4,5,5-Tetramethyl-2-(3-(propoxy-d7)phenyl)-1,3,2-dioxaborolane (P6a)

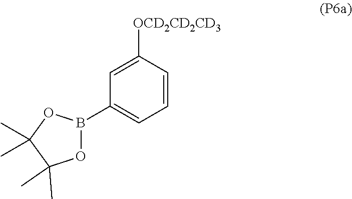

A solution of 3-(4,4,5,5-tetramethyl-1,3,2-dioxaborolan-2-yl)phenol (200 mg) and K₂CO₃ (376 mg) in MeCN (2 mL) was added C₃D₇I (241 mg). The mixture was stirred at 60° C. overnight, cooled, diluted with water and extracted with EtOAc (3×). The combined organic layer was separated, dried over Na₂SO₄, filtered, concentrated and purified by FCC (PE:EA=20:1) to give compound P6a as a colorless oil. LCMS (ESI): m/z 270.3 (M+H)⁺.

### Step 2: 2,3,5,6-Tetrafluoro-3′-(propoxy-d7)-[1,1′-biphenyl]-4-amine (P6)

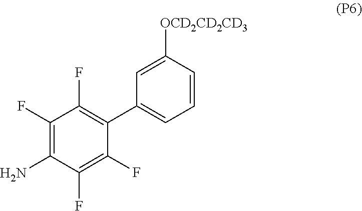

To a solution of compound P6a (150 mg) in 1,4-dioxane (2 mL) and H₂O (0.2 mL) was added 4-bromo-2,3,5,6-tetrafluoroaniline (136 mg), Na₂CO₃ (177 mg) and Pd(dppf)Cl₂ (15 mg). The mixture was heated at 90° C. for 8 h, cooled, diluted with water and extracted with EtOAc (3×). The combined organic layer was separated, dried over Na₂SO₄, filtered, concentrated and purified by FCC (PE:EA=8:1) to give compound P6 as a colorless oil. LCMS (ESI): m/z 307.1 (M+H)⁺.

### Preparative Example P6/1 to P6/4

The following Examples were prepared similar as described for Preparative Example 6 above using the appropriate building block(s) as shown below.

| # | building block(s) | structure |
|---|---|---|
| P6/1 | ICD₂CD₂CD₂CD₃ | 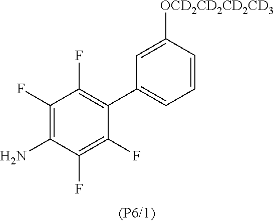 |
| P6/2 | 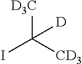 [SMILES: [2H]C([2H])([2H])C([2H])(I)C([2H])([2H])[2H]] | 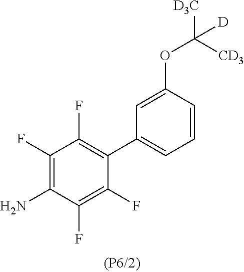 [SMILES: [2H]C([2H])([2H])C([2H])(Oc1cccc(-c2c(F)c(F)c(N)c(F)c2F)c1)C([2H])([2H])[2H]] |
| P6/3 | 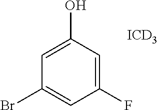 | 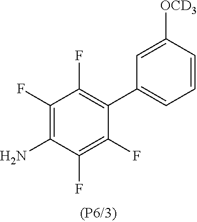 |
| P6/4 | ICD₂CD₃ | 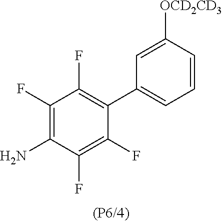 |

### Preparative Example P7

### 5-Fluorothiophene-2,3-dicarboxylic acid (P7)

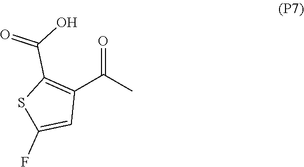

A solution of 5-fluorothiophene-2,3-dicarbaldehyde (400 mg) in tert-butanol (4 mL) and H₂O (1 mL) was added NaClO₂ (3.4 g) and NaHPO₄ (600 mg). The mixture was stirred at rt for 1 h, concentrated and purified by reversed-phase flash chromatography (C18) (0.1% TFA in water, 10 to 100% MeCN) to give compound P7 as a white solid. LCMS (ESI): m/z 191.1 (M+H)⁺.

### Example 1

### N¹-(Methylsulfonyl)-N²-(2,3,5,6-tetrafluoro-3′-(trifluoromethoxy)-[1,1′-biphenyl]-4-yl)cyclopent-1-ene-1,2-dicarboxamide (1)

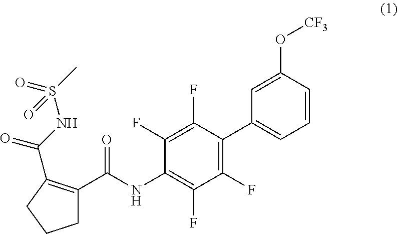 [SMILES: CS(=O)(=O)NC(=O)C1=C(C(=O)Nc2c(F)c(F)c(-c3cccc(OC(F)(F)F)c3)c(F)c2F)CCC1]

To a solution of 2-((2,3,5,6-tetrafluoro-3′-(trifluoromethoxy)-[1,1′-biphenyl]-4-yl)carbamoyl)cyclo-pent-1-ene-1-carboxylic acid (120 mg, 0.25 mmol) in DCM (5 mL) was added methanesulfonamide (37 mg, 0.38 mmol), dicyclohexylcarbodiimide (80 mg, 0.38 mmol), 4-dimethylaminopyridine (32 mg, 0.25 mmol) and TEA (79 μL, 0.77 mmol). The mixture was stirred at 60° C. for 8 h in a sealed tube, then cooled to rt and diluted with water (5 mL). The organic layer was separated, concentrated and then purified by prep. HPLC to afford compound 1 as a white solid. ¹H-NMR (400 MHz, MeOD-d4) δ 7.63 (t, J=8.2 Hz, 1H), 7.52 (d, J=8.0 Hz, 1H), 7.45-7.41 (m, 2H), 3.12 (s, 3H), 2.94-2.86 (i, 4H), 1.97-1.85 (m, 2H). LCMS (ESI): m/z 541.2 (M+H)⁺.

### Example 1/1 to 1/14

The following Examples were prepared similar as described for Example 1 above using the appropriate building block(s) as shown below. The acid intermediate can be prepared as outlined in Example 4.

| # | building block(s) | structure | analytical data |
|---|---|---|---|
| 1/1 | acid (4c) | 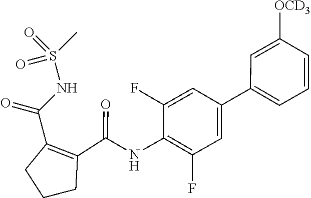 | ¹H-NMR (500 MHz, MeOD-d4) δ 7.38- 7.32 (m, 3H), 7.20 (dd, J = 1.0, 8.0 Hz, 1H), 7.17-7.16 (m, 1H), 6.96 (dd, J = 2.7, 8.3 Hz, 1H), 3.18 (s, 3H), 2.92-2.86 (m, 4H), 2.01-1.95 (m, 2H). LCMS (ESI): m/z 544.3 (M + H)⁺ |
| 1/2 | 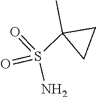 | 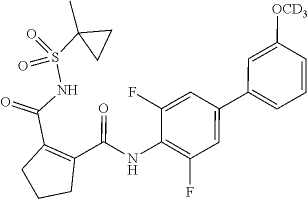 | ¹H-NMR (500 MHz, MeOD-d4) δ 7.38- 7.32 (m, 3H), 7.20 (d, J = 8.0 Hz, 1H), 7.16 (t, J = 2.0 Hz, 1H), 6.96 (dd, J = 2.3, 8.3 Hz, 1H), 2.97-2.85 (m, 4H), 2.04-1.97 (m, 2H), 1.51 (s, 5H), 0.83 (br s, 2H). LCMS (ESI): m/z 494.1 (M + H)⁺ |
| | acid (4c), | | |
| 1/3 | 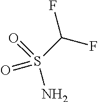 | 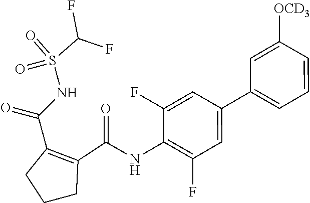 | ¹H-NMR (500 MHz, MeOD-d4) δ 7.39- 7.33 (m, 3H), 7.21 (d, J = 8.5 Hz, 1H), 7.17 (t, J = 2.5 Hz, 1H), 6.98- 6.96 (m, 1H), 6.78 (t, J = 53.5 Hz, 1 H), 2.98 (t, J = 7.3 Hz, 2H), 2.85 (t, J = 7.5 Hz, 2H), 2.12-2.04 (m, 2H). LCMS (ESI): m/z 490.1 (M + H)⁺, 512.3 (M + Na)⁺ |
| | acid (4c), | | |
| 1/4 | 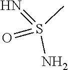 | 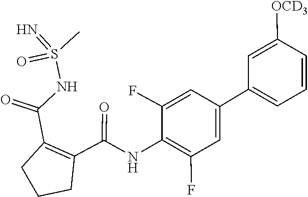 | ¹H-NMR (400 MHz, MeOD-d4) δ 7.38- 7.32 (m, 3H), 7.20 (d, J = 8.0 Hz, 1H), 7.16 (t, J = 2.0 Hz, 1H), 6.96 (dd, J = 2.2, 8.2 Hz, 1H), 3.35 (s, 3H), 2.92-2.84 (m, 4H), 1.95-1.87 (m, 2H). LCMS (ESI): m/z 453.2 (M + H)⁺ |
| | acid (4c), | | |
| 1/5 | 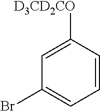 | 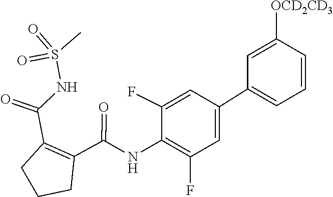 | ¹H-NMR (500 MHz, MeOD-d4) δ 7.38- 7.32 (m, 3H), 7.20 (d, J = 7.5 Hz, 1H), 7.15 (t, J = 2.0 Hz, 1H), 6.95 (dd, J = 2.0, 8.0 Hz, 1H), 3.27 (s, 3H), 2.96 (t, J = 7.5 Hz, 2H), 2.84 (t, J = 7.5 Hz, 2H), 2.13-2.07 (m, 2H). LCMS (ESI): m/z 470.3 (M + H)⁺ |
| 1/6 | 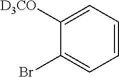 | 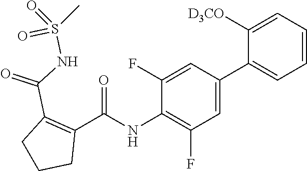 | ¹H-NMR (400 MHz, MeOD-d4) δ 7.38- 7.32 (m, 2H), 7.22- 7.18 (m, 2H), 7.08 (d, J = 8.4 Hz, 1H), 7.02 (dd, J = 7.2, 7.6 Hz, 1H), 3.17 (s, 3H), 2.92- 2.85 (m, 4H), 2.02- 1.94 (m, 2H). LCMS (ESI): m/z 454.2 (M + H)⁺ |
| 1/7 | 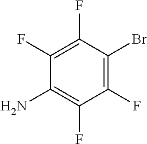 [SMILES: Nc1c(F)c(F)c(Br)c(F)c1F] | 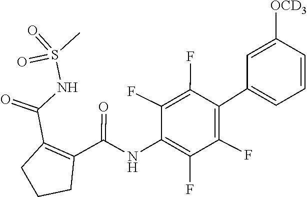 | ¹H-NMR (400 MHz, MeOD-d4) δ 7.42 (t, J = 7.6 Hz, 1H), 7.05- 7.02 (m, 3H), 3.10 (s, 3H), 2.95-2.84 (m, 4H), 1.88 (q, J = 7.6 Hz, 2H). LCMS (ESI): m/z 490.1 (M + H)⁺ |
| 1/8 | 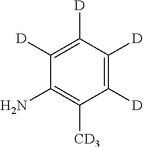 [SMILES: [2H]c1c([2H])c([2H])c(C([2H])([2H])[2H])c(N)c1[2H]] | 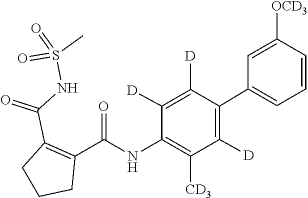 | ¹H-NMR (400 MHz, DMSO-d6) δ 12.42 (br s, 1H), 9.59 (s, 1H), 7.37 (t, J = 8.2 Hz, 1H), 7.24-7.18 (m, 2H), 6.94-6.91 (m, 1H), 3.26 (s, 3H), 2.91-2.88 (m, 2H), 2.76-2.72 (m, 2H), 2.00-1.92 (m, 2H). LCMS (ESI): m/z 438.3 (M + H)⁺ |
| 1/9 | 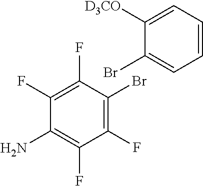 | 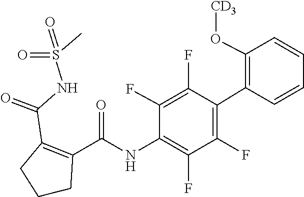 [SMILES: [2H]C([2H])([2H])Oc1ccccc1-c1c(F)c(F)c(NC(=O)C2=C(C(=O)NS(C)(=O)=O)CCC2)c(F)c1F] | ¹H-NMR (400 MHz, MeOD-d4) δ 7.51- 7.45 (m, 1H), 7.28- 2.26 (m, 1H), 7.13 (d, J = 7.6 Hz, 1H), 7.08- 7.04 (m, 1H), 3.07 (s, 3H), 2.96-2.91 (m, 2H), 2.88-2.83 (m, 2H), 1.88-1.81 (m, 2H). LCMS (ESI): m/z 490.1 (M + H)⁺ |
| 1/10 | 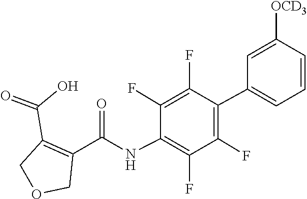 | 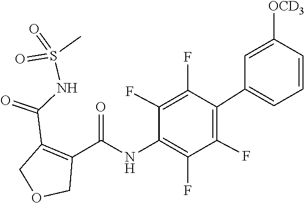 | ¹H-NMR (400 MHz, MeOD-d4) δ 7.44-7.40 (m, 1H), 7.05-7.03 (m, 3H), 5.09-5.03 (m, 4H), 3.08 (s, 3H). LCMS (ESI): m/z 509.1 (M + H)⁺ |
| 1/11 | 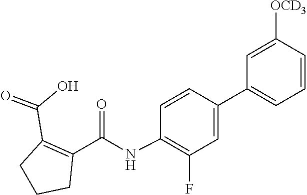 | 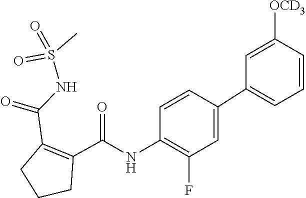 | ¹H-NMR (400 MHz, DMSO-d6) δ 12.09 (br s, 1H), 10.02 (br s, 1H), 7.79 (t, J = 8.2 Hz, 1H), 7.64 (dd, J = 1.8, 12.2 Hz, 1H), 7.53 (dd, J = 1.6, 8.4 Hz, 1H), 7.38 (t, J = 8.0 Hz, 1H), 7.28-7.23 (m, 2H), 6.94 (dd, J = 1.8, 6.6 Hz, 1H), 3.24 (s, 3H), 2.84 (t, |
| | | | J = 7.2 Hz, 2H), |
| | | | 2.74 (t, J = 7.8 Hz, |
| | | | 2H), 1.99-1.91 (m, |
| | | | 2H). LCMS (ESI): |
| | | | m/z 436.1 (M + H)⁺ |
| 1/12 | 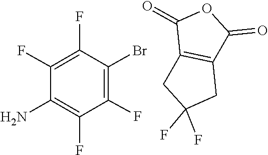 | 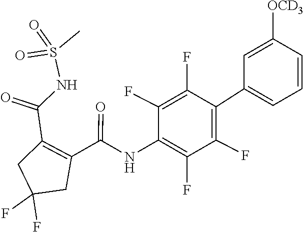 | ¹H-NMR (400 MHz, MeOD-d4) δ 7.43 (t, J = 8.2 Hz, 1H), 7.07- 7.03 (m, 3H), 3.51-3.28 (m, 7H). LCMS (ESI): m/z 526.0 (M + H)⁺ |
| 1/13 | 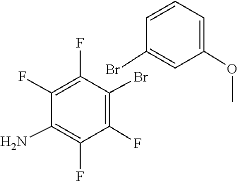 | 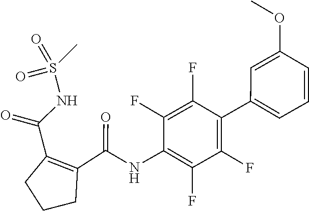 [SMILES: COc1cccc(-c2c(F)c(F)c(NC(=O)C3=C(C(=O)NS(C)(=O)=O)CCC3)c(F)c2F)c1] | LCMS (ESI): m/z 487.1 (M + H)⁺ |
| 1/14 | 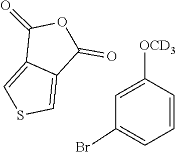 | 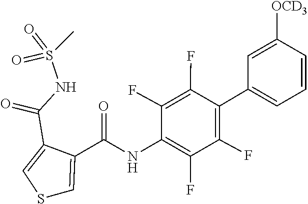 | ¹H-NMR (400 MHz, DMSO-d6) δ 12.31 (br s, 1H), 10.82 (s, 1H), 8.32 (d, J = 2.4 Hz, 1H), 8.25 (d, J = 3.2 Hz, 1H), 7.48 (t, J = 7.8 Hz, 1H), 7.16- 7.08 (m, 3H), 3.31 (s, 3H). LCMS (ESI): m/z 506.0 (M + H)⁺ |

### Example 2

### N¹-Sulfamoyl-N²-(2,3,5,6-tetrafluoro-3′-(trifluoromethoxy)-[1,1′-biphenyl]-4-yl)cyclopent-1-ene-1,2-dicarboxamide (2)

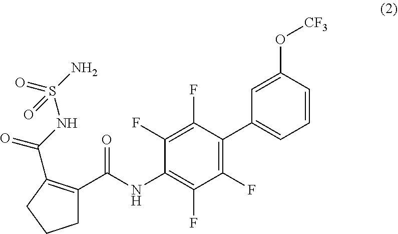 [SMILES: NS(=O)(=O)NC(=O)C1=C(C(=O)Nc2c(F)c(F)c(-c3cccc(OC(F)(F)F)c3)c(F)c2F)CCC1]

To a solution of 2-((2,3,5,6-tetrafluoro-3′-(trifluoromethoxy)-[1,1′-biphenyl]-4-yl)carbamoyl)cyclo-pent-1-ene-1-carboxylic acid (300 mg, 0.65 mmol) in DCM (5 mL) was added sulfuric diamide (124 mg, 1.30 mmol), dicyclohexylcarbodiimide (200 mg, 0.97 mmol), 4-dimethylaminopyridine (79 mg, 0.65 mmol) and TEA (107 μL, 0.77 mmol). The mixture was stirred at 60° C. for 8 h in a sealed tube, then cooled to rt and diluted with water (5 mL). The organic layer was separated, concentrated and then purified by prep. HPLC to afford compound 2 as a white solid. ¹H-NMR (400 MHz, MeOD-d4) δ 7.64 (t, J=8.0 Hz, 1H), 7.53 (d, J=8.0 Hz, 1H), 7.46-7.43 (m, 2H), 2.99-2.93 (m, 2H), 2.88-2.83 (m, 2H), 2.13-2.04 (in, 2H). LCMS (ESI): m/z 542.2 (M+H)⁺.

### Example 2/1 to 2/6

The following Examples were prepared similar as described for Example 2 above using the appropriate building block(s) as shown below. The acid intermediate can be prepared as outlined in Example 4.

| # | building block(s) | structure | analytical data |
|---|---|---|---|
| 2/1 | acid (4c) | 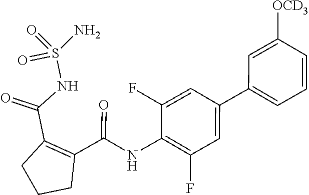 | ¹H-NMR (500 MHz, MeOD-d4) δ 7.36 (t, J = 8.0 Hz, 1H), 7.31 (d, J = 9.0 Hz, 2H), 7.19 (d, J = 7.5 Hz, 1H), 7.16 (t, J = 2.0 Hz, 1H), 6.95 (dd, J = 2.3, 8.3 Hz, 1H), 2.94-2.84 (m, 4H), 1.92-1.84 (m, 2H). LCMS (ESI): m/z 455.2 (M + H)⁺ |
| 2/2 | 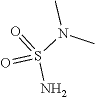 | 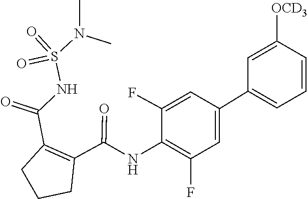 | ¹H-NMR (500 MHz, MeOD-d4) δ 7.36 (t, J = 8.0 Hz, 1H), 7.31 (d, J = 9.0 Hz, 2H), 7.19 (d, J = 7.5 Hz, 1H), 7.16 (d, J = 1.8 Hz, 1H), 6.95 (dd, J = 2.8, 8.3 Hz, 1H), 2.94-2.85 (m, 4H), 2.81 (s, 6H), 1.94-1.87 (m, 2H). LCMS (ESI): m/z 483.3 (M + H)⁺ |
| | acid (4c), | | |
| 2/3 | 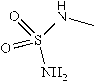 | 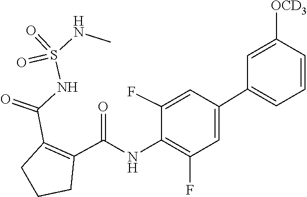 | ¹H-NMR (500 MHz, MeOD-d4) δ 7.39-7.33 (m, 3H), 7.21 (d, J = 8.0 Hz, 1H), 7.17 (d, J = 2.0 Hz, 1H), 6.97 (dd, J = 1.8, 8.3 Hz, 1H), 2.97 (t, J = 7.3 Hz, 2H), 2.85 (t, J = 7.3 Hz, 2H), 2.67 (s, 3H), 2.11-2.03 (m, 2H). LCMS (ESI): m/z 469.3 (M + H)⁺ |
| | acid (4c), | | |
| 2/4 | 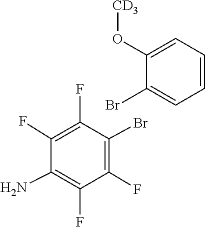 | 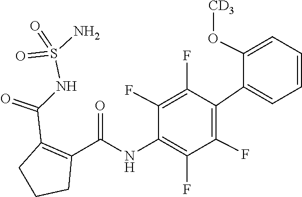 [SMILES: [2H]C([2H])([2H])Oc1ccccc1-c1c(F)c(F)c(NC(=O)C2=C(C(=O)NS(N)(=O)=O)CCC2)c(F)c1F] | ¹H-NMR (400 MHz, MeOD-d4) δ 7.50-7.46 (m, 1H), 7.27 (dd, J = 1.0, 7.4 Hz, 1H), 7.14 (d, J = 8.8 Hz, 1H), 7.09-7.05 (m, 1H), 2.95- 2.86 (m, 4H), 2.05-1.97 (m, 2H). LCMS (ESI): m/z 491.1 (M + H)⁺ |
| 2/5 | 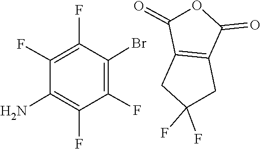 | 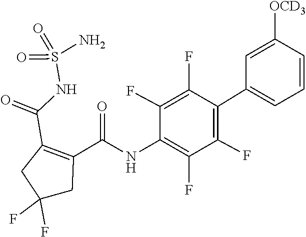 | ¹H-NMR (400 MHz, MeOD-d4) δ 7.43 (t, J = 7.8 Hz, 1H), 7.07-7.02 (m, 3H), 3.53-3.30 (m, 4H). LCMS (ESI): m/z 526.9 (M + H)⁺ |
| 2/6 | 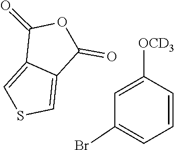 | 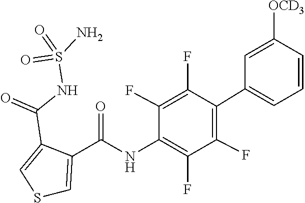 | ¹H-NMR (400 MHz, DMSO-d6) δ 12.06 (s, 1H), 10.89 (s, 1H), 8.28 (dd, J = 3.2, 9.6 Hz, 2H), 7.53-7.45 (m, 3H), 7.15-7.08 (m, 3H). LCMS (ESI): m/z 507.0 (M + H)⁺ |

### Example 2-1 (Alternative Synthesis of N-Sulfamoylamides)

### N³-(3,5-Difluoro-3′-(methoxy-d3)-[1,1′-biphenyl]-4-yl)-N⁴-sulfamoyl-2,5-dihydrofuran-3,4-di-carboxamide (2-1)

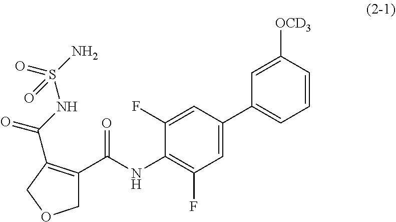

To a solution of 5e (200 mg) in dry DCM (5 mL) was added SOCl₂ (120 mg) at 0° C. The mixture was stirred at 0° C. for 2 h and concentrated under vacuum to afford the crude acid chloride intermediate. A solution of sulfuric diamide (457 mg) in dry DMF (5 mL) was added to the acid chloride intermediate and DIPEA (136 mg) at 0° C. The mixture was stirred at 90° C. for 16 h, concentrated and purified by preparative HPLC to afford compound 2-1 as a white solid. ¹H-NMR (400 MHz, MeOD-d4) δ 7.40-7.36 (m, 3H), 7.21 (d, J=7.6 Hz, 1H), 7.18 (t, J=2.0 Hz, 1H), 6.98 (dd, J=2.4, 8.0 Hz, 1H), 5.20-5.17 (m, 2H), 5.06-5.03 (m, 2H). LCMS (ESI): m/z 457.1 (M+H)⁺.

### Example 2-1/1

The following Example was prepared similar as described for Example 2-1 above using the appropriate building block(s) as shown below.

| # | building block | structure | analytical data |
|---|---|---|---|
| 2-1/1 | 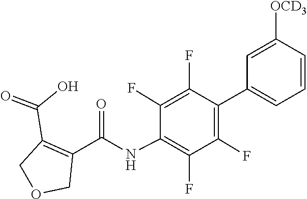 | 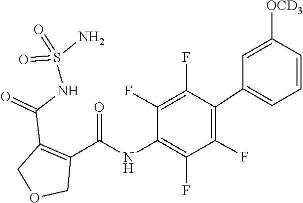 | ¹H-NMR (400 MHz, MeOD-d4) δ 7.45- 7.41 (m, 1H), 7.07- 7.04 (m, 3H), 5.19- 5.16 (m, 2H), 5.06- 5.04 (m, 2H). LCMS (ESI): m/z 493.1 (M + H)⁺ |

### Example 3

### Step 1: N-(2,3,5,6-Tetrafluoro-3′-(trifluoromethoxy)-[1,1′-biphenyl]-4-yl)cyclopent-1-ene-1,2-dicarboxamide (3a)

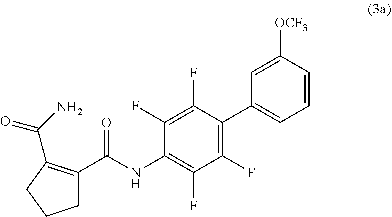 [SMILES: NC(=O)C1=C(C(=O)Nc2c(F)c(F)c(-c3cccc(OC(F)(F)F)c3)c(F)c2F)CCC1]

To a solution 2-((2,3,5,6-tetrafluoro-3′-(trifluoromethoxy)-[1,1′-biphenyl]-4-yl)carbamoyl)cyclopent-1-ene-1-carboxylic acid (500 mg, 1.08 mmol) in DMF (10 mL) was added NH₄Cl (114 mg, 2.16 mmol), EDCI (207 mg, 1.08 mmol) and 4-dimethylaminopyridine (145 mg, 1.19 mmol). The mixture was heated at 60° C. for 3 hour, concentrated and purified by FCC (PE:EA=5:1) to give compound 3a as a white solid. LCMS (ESI): m/z 463.0 (M+H)⁺.

### Step 2: 2-Cyano-N-(2,3,5,6-tetrafluoro-3′-(trifluoromethoxy)-[1,1′-biphenyl]-4-yl)cyclopent-1-ene-1-carboxamide (3b)

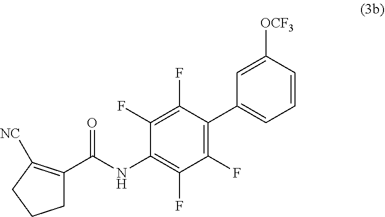 [SMILES: N#CC1=C(C(=O)Nc2c(F)c(F)c(-c3cccc(OC(F)(F)F)c3)c(F)c2F)CCC1]

To a solution of compound 3a (400 mg, 0.87 mmol) in DMF (5 mL) was added cyanuric chloride (320 mg, 1.73 mmol) at 0° C. for 30 minute and then the mixture was stirred at rt for 3 h, concentrated and purified by FCC (PE:EA=5:1) to afford compound 3b as a white solid. LCMS (ESI): m/z 445.3 (M+H)⁺.

### Step 3: N-(2,3,5,6-Tetrafluoro-3′-(trifluoromethoxy)-[1,1′-biphenyl]-4-yl)-2-(2H-tetrazol-5-yl)cyclopent-1-ene-1-carboxamide (3)

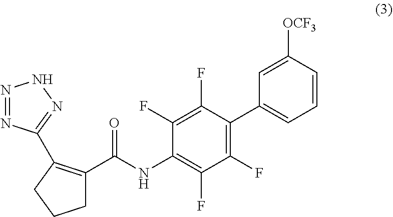 [SMILES: O=C(Nc1c(F)c(F)c(-c2cccc(OC(F)(F)F)c2)c(F)c1F)C1=C(c2nn[nH]n2)CCC1]

To a solution of compound 3b (180 mg, 0.41 mmol) in 1,2-dimethoxyethane (4 mL) and H₂O (2 mL) was added ZnBr₂ (100 mg, 0.45 mmol) and NaN₃ (79 mg, 1.23 mmol). The mixture was heated at 105° C. for 3 h, cooled to rt, concentrated and purified by prep. HPLC to afford compound 3 as a white solid. ¹H-NMR (500 MHz, MeOD-d4) δ 7.64 (t, J=8.0 Hz, 1H), 7.54 (d, J=7.5 Hz, 1H), 7.47 (s, 1H), 7.42 (d, J=8.0 Hz, 1H), 3.31-3.29 (m, 2H), 3.00 (t, J=7.5 Hz, 2H), 2.05-1.98 (m, 2H). LCMS (ESI): m/z 488.2 (M+H)⁺.

### Example 3/1

The following Example was prepared similar as described for Example 3 above using the appropriate carboxylic acid building block.

| # | structure | analytical data |
|---|---|---|
| 3/1 | 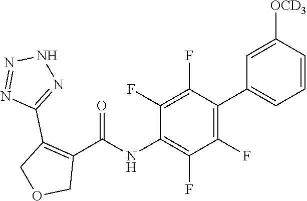 | ¹H-NMR (400 MHz, MeOD-d4) δ 7.45-7.41 (m, 1H), 7.07-7.03 (m, 3H), 5.43-5.40 (m, 2H), 5.15-5.12 (m, 2H). LCMS (ESI): m/z 438.8 (M + H)⁺ |

### Example 3-1

### Step 1: 2-Cyano-N-(3,5-difluoro-3′-(methoxy-d3)-[1,1′-biphenyl]-4-yl)cyclopent-1-ene-1-carboxamide (3-1a)

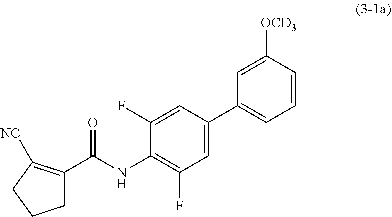

Compound 3-1a was prepared starting with acid 4c similar as described in Example 3, step 1 and 2. LCMS (ESI): m/z 358.2 (M+H)⁺.

### Step 2: (Z)—N-(3,5-Difluoro-3′-(methoxy-d3)-[1,1′-biphenyl]-4-yl)-2-(N-hydroxycarbamimid-oyl)cyclopent-1-ene-1-carboxamide (3-1)

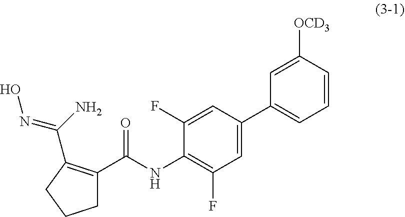

A solution of compound 3-1a (200 mg, 0.56 mmol) in MeOH (5 mL) was added hydroxylamine hydrochloride (58 mg) and DIPEA (108 mg). The mixture was stirred at 60° C. for overnight, concentrated and purified by prep. HPLC to afford target molecule 3-1 as a white solid. ¹H-NMR (400 MHz, DMSO-d6) δ 9.94 (s, 1H), 7.60-7.55 (m, 2H), 7.40 (t, J=8.0 Hz, 1H), 7.33-7.29 (m, 2H), 6.06 (s, 2H), 6.99 (dd, J=2.2, 8.0 Hz, 1H), 2.86-2.74 (m, 4H), 1.87-1.78 (m, 2H). LCMS (ESI): m/z 391.1 (M+H)⁺.

### Example 3-2

### N-(3,5-Difluoro-3′-(methoxy-d3)-[1,1′-biphenyl]-4-yl)-2-(5-oxo-4,5-dihydro-1,2,4-oxadiazol-3-yl)cyclopent-1-ene-1-carboxamide (3-2)

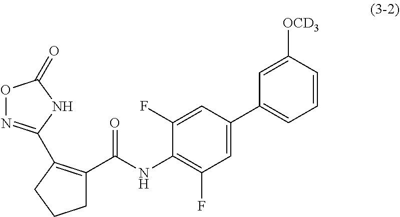

A solution of compound 3-1a (120 mg, 0.31 mmol) in 1,4-dioxane (5 mL) was added 1,1′-carbonyldiimidazole (55 mg) and 1,8-diazabicyclo(5.4.0)undec-7-ene (56 mg). The mixture was stirred at 100° C. for 2 h, cooled to rt, concentrated and purified by prep. HPLC to yield target molecule 3-2 as a white solid. ¹H-NMR (500 MHz, DMSO-d6) δ 12.25 (br s, 1H), 10.01 (br s, 1H), 7.58 (t, J=9.0 Hz, 2H), 7.40 (t, J=8.3 Hz, 1H), 7.33-7.29 (m, 2H), 6.99 (dd, J=1.8, 8.3 Hz, 1H), 2.94 (t, J=6.8 Hz, 2H), 2.83 (t, J=7.4 Hz, 2H), 2.07-1.99 (m, 2H). LCMS (ESI): m/z 417.1 (M+H)⁺.

### Example 4

### Step 1: 2,6-Difluoro-4-(4,4,5,5-tetramethyl-1,3,2-dioxaborolan-2-yl)aniline (4a)

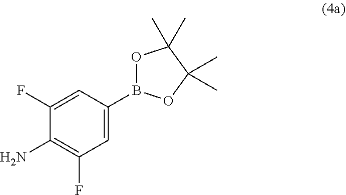 [SMILES: CC1(C)OB(c2cc(F)c(N)c(F)c2)OC1(C)C]

To a solution of 4-bromo-2,6-difluoroaniline (10 g, 48 mmol) in 1,4-dioxane (100 mL) was added bis(pinacolato)diboron (12.8 g, 50.4 mmol), CH₃COOK (14.1 g, 144 mmol) and Pd(dppf)Cl₂ (1.0 g, 2.40 mmol). The mixture was stirred at 90° C. under N₂ for 2 h, cooled to rt, concentrated and purified by FCC (PE:EA=10:1) to give compound 4a as a yellow solid.

### Step 2: 3,5-Difluoro-3′-(methoxy-d3)-[1,1′-biphenyl]-4-amine (4b)

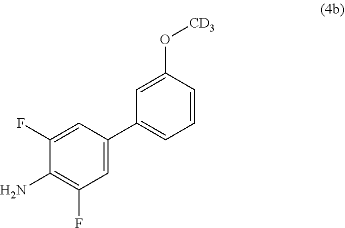 [SMILES: [2H]C([2H])([2H])Oc1cccc(-c2cc(F)c(N)c(F)c2)c1]

To a solution of compound 4a (4.50 g, 13.3 mmol) in 1,4-dioxane (50 mL) and H₂O (5 mL) was added 1-bromo-3-(methoxy-d3)benzene (3.34 g, 13.3 mmol), Na₂CO₃ (5.61 g, 39.4 mmol) and Pd(dppf)Cl₂ (400 mg, 0.67 mmol). The mixture was stirred at 90° C. under N₂ for 2 h, cooled to rt, concentrated and purified by FCC (PE:EA=10:1) to give compound 4b as a yellow solid. LCMS (ESI): m/z 239.1 (M+H)⁺.

### Step 3: 2-((3,5-Difluoro-3′-(methoxy-d3)-[1,1′-biphenyl]-4-yl)carbamoyl)cyclopent-1-ene-1-carboxylic acid (4c)

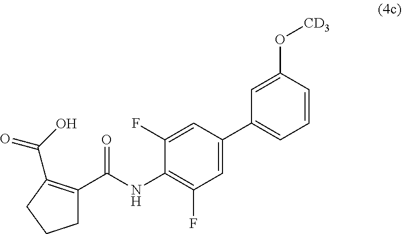 [SMILES: [2H]C([2H])([2H])Oc1cccc(-c2cc(F)c(NC(=O)C3=C(C(=O)O)CCC3)c(F)c2)c1]

To a solution of compound 4b (3.40 g, 14.3 mmol) in DCM (20 mL) was added 1-cyclopentene-1,2-dicarboxylic anhydride (1.90 g, 14.3 mmol) and then the mixture was stirred at rt for 2 h. The mixture was filtered and the filter cake washed with MeCN. The solid was dried in vacuum to afford compound 4c as a white solid. LCMS (ESI): m/z 377.3 (M+H)⁺.

### Step 4: N¹-(3,5-Difluoro-3′-(methoxy-d3)-[1,1′-biphenyl]-4-yl)-N-methoxycyclopent-1-ene-1,2-dicarboxamide (4)

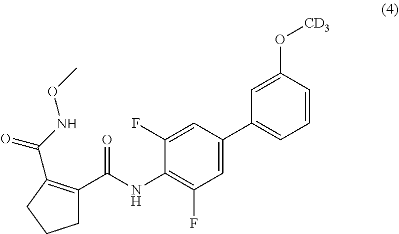 [SMILES: [2H]C([2H])([2H])Oc1cccc(-c2cc(F)c(NC(=O)C3=C(C(=O)NOC)CCC3)c(F)c2)c1]

To a solution of compound 4c (300 mg, 0.80 mmol) in DCM (25 mL) was added O-methylhydroxylamine (132 mg, 2.80 mmol), dicyclohexylcarbodiimide (246 mg, 1.14 mmol), 4-dimethylaminopyridine (97 mg, 0.79 mmol) and TEA (0.30 mL, 2.2 mmol). Then mixture was stirred at 60° C. for 8 h in a sealed tube, cooled to rt, concentrated and purified by prep. HPLC to afford compound 4 as a white solid. ¹H-NMR (500 MHz, MeOD-d4) δ 7.40-7.32 (m, 3H), 7.20 (d, J=8.0 Hz, 1H), 7.16 (s, 1H), 6.97 (dd, J=8.3, 2.3 Hz, 1H), 3.75 (s, 3H), 2.92-2.88 (m, 2H), 2.82-2.78 (m, 2H), 2.06-2.00 (m, 2H). LCMS (ESI): m/z 406.2 (M+H)⁺.

### Example 4/1 to 4/35

The following Examples were or can be prepared similar as described for Example 4 above using the appropriate building block(s) as shown below.

| # | building block(s) | structure | analytical data |
|---|---|---|---|
| 4/1 | 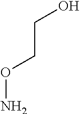 | 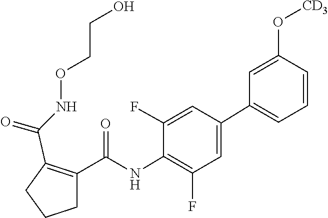 [SMILES: [2H]C([2H])([2H])Oc1cccc(-c2cc(F)c(NC(=O)C3=C(C(=O)NOCCO)CCC3)c(F)c2)c1] | ¹H-NMR (500 MHz, MeOD-d4) δ 7.39- 7.33 (m, 3H), 7.20 (d, J = 8.0 Hz, 1H), 7.16 (t, J = 2.0 Hz, 1H), 6.96 (dd, J = 8.3, 2.3 Hz, 1H), 4.00-3.97 (m, 2H), 3.75-3.72 (m, 2H), 2.92-2.88 (m, 2H), 2.82-2.79 (m, 2H), 2.07-2.00 (m, 2H). LCMS (ESI): m/z 436.2 (M + H)⁺ |
| 4/2 | 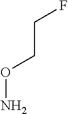 | 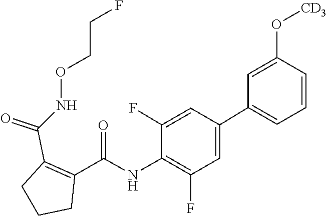 [SMILES: [2H]C([2H])([2H])Oc1cccc(-c2cc(F)c(NC(=O)C3=C(C(=O)NOCCF)CCC3)c(F)c2)c1] | ¹H-NMR (400 MHz, MeOD-d4) δ 7.39- 7.32 (m, 3H), 7.22- 7.16 (m, 2H), 6.98- 7.95 (m, 1H), 4.68 (t, J = 3.8 Hz, 1H), 4.56 (t, J = 4.0 Hz, 1H), 4.19 (t, J = 3.8 Hz, 1H), 4.12 (t, J = 4.5 Hz, 1H), 2.93- 2.77 (m, 4H), 2.09- 2.03 (m, 2H). LCMS (ESI): m/z 438.2 (M + H)⁺ |
| 4/3 | 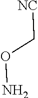 | 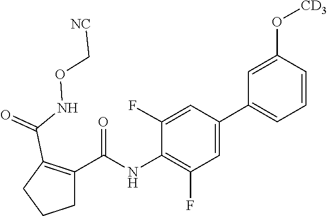 [SMILES: [2H]C([2H])([2H])Oc1cccc(-c2cc(F)c(NC(=O)C3=C(C(=O)NOCC#N)CCC3)c(F)c2)c1] | ¹H-NMR (400 MHz, DMSO-d6) δ 11.75 (br s, 1H), 9.93 (br s, 1H), 7.52- 7.48 (m, 2H), 7.39 (t, J = 8.0 Hz, 1H), 7.30-7.26 (m, 2H), 6.98 (dd, J = 2.0, 8.0 Hz, 1H), 4.80 (s, 2H), 2.83-2.71 (m, 4H), 1.98-1.91 (m, 2H). LCMS (ESI): m/z 431.2 (M + H)⁺ |
| 4/4 | 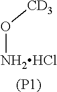 | 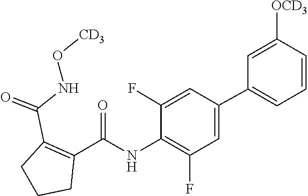 | |
| 4/5 | 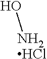 | 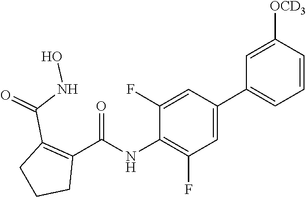 | ¹H-NMR (400 MHz, DMSO-d6) δ 11.43 (s, 1H), 11.16 (s, 1H), 9.35 (s, 1H), 7.57 (d, J = 8.8 Hz, 2H), 7.39 (t, J = 8.0 Hz, 1H), 7.33- 7.28 (m, 2H), 6.98 (dd, J = 1.8, 8.2 Hz, 1H), 2.79-2.75 (m, 4H), 1.90-1.82 (m, 2H). LCMS (ESI): m/z 492.2 (M + H)⁺ |
| 4/6 | 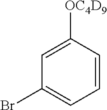 | 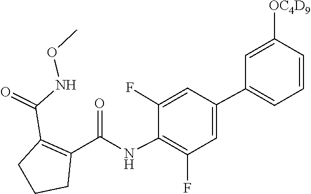 | |
| 4/7 | 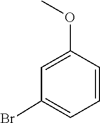 | 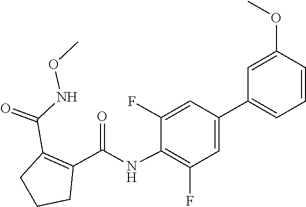 [SMILES: CONC(=O)C1=C(C(=O)Nc2c(F)cc(-c3cccc(OC)c3)cc2F)CCC1] | |
| 4/8 | 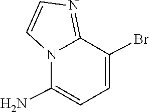 [SMILES: Nc1ccc(Br)c2nccn12] | 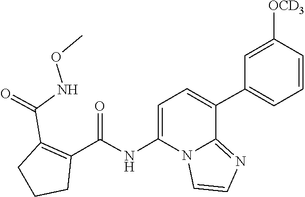 | ¹H-NMR (400 MHz, MeOH-d4) δ 8.27 (s, 1H), 7.64 (t, J = 4.4 Hz, 2H), 7.43- 7.32 (m, 2H), 7.02- 6.94 (m, 3H), 3.73 (s, 3H), 2.72-2.61 (m, 4H), 2.03-1.96 (m, 2H). LCMS (ESI): m/z 410.0 (M + H)⁺ |
| 4/9 | 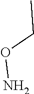 | 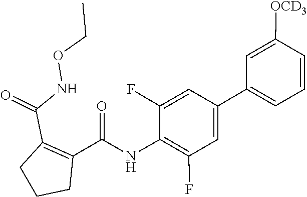 | ¹H-NMR (500 MHz, DMSO-d6) δ 11.32 (s, 1H), 10.44 (s, 1H), 7.56 (d, J = 9.0 Hz, 2H), 7.39 (t, J = 7.5 Hz, 1H), 7.33-7.29 (m, 2H), 6.99 (dd, J = 2.3, 8.3 Hz, 1H), 3.88-3.83 (m, 2H), 2.80-2.68 (m, 4H), 1.94-1.88 (m, 2H), 1.15 (t, (J = 6.8 Hz, 3H). LCMS |
| | | | (ESI): m/z 419.8 |
| | | | (M + H)⁺ |
| 4/10 | 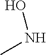 | 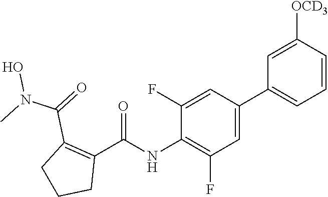 | ¹H-NMR (500 MHz, DMSO-d6) δ 9.94 (br s, 1H), 9.41 (s, 1H), 7.56 (d, J = 9.0 Hz, 2H), 7.39 (t, J = 7.8 Hz, 1H), 7.31 (d, J = 7.5 Hz, 1H), 7.28 (s, 1H), 6.98 (dd, J = 2.0, 8.0 Hz, 1H), 3.11 (s, 3H), 2.77-2.66 (m, 4H), 2.01-1.95 (m, 2H). LCMS (ESI): m/z 405.9 (M + H)⁺ |
| 4/11 | 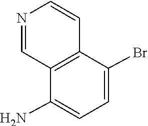 [SMILES: Nc1ccc(Br)c2ccncc12] | 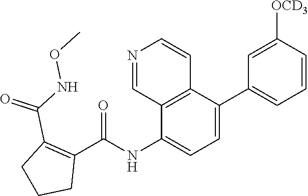 | ¹H-NMR (500 MHz, MeOH-d4) δ 9.58 (s, 1H), 8.43 (d, J = 5.5 Hz, 1H), 8.00 (d, J = 7.5 Hz, 1H), 7.81 (d, J = 6.0 Hz, 1H), 7.74 (d, J = 7.5 Hz, 1H), 7.44 (J = 7.8 Hz, 1H), 7.05- 7.01 (m, 3H), 3.76 (s, 3H), 2.98-2.94 (m, 2H), 2.85-2.81 (m, 2H), 2.09-2.02 (m, |
| | | | 2H). LCMS (ESI): |
| | | | m/z 421.2 (M + H)⁺ |
| 4/12 | 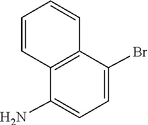 [SMILES: Nc1ccc(Br)c2ccccc12] | 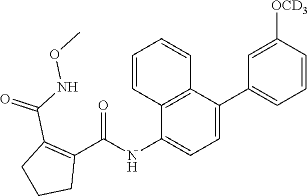 | ¹H-NMR (500 MHz, MeOH-d4) δ 9.58 (s, 1H), 8.43 (d, J = 5.5 Hz, 1H), 8.00 (d, J = 7.5 Hz, 1H), 7.81 (d, J = 6.0 Hz, 1H), 7.74 (d, J = 7.5 Hz, 1H), 7.44 (J = 7.8 Hz, 1H), 7.05- 7.01 (m, 3H), 3.76 (s, 3H), 2.98-2.94 (m, 2H), 2.85-2.81 (m, 2H), 2.09-2.02 (m, |
| | | | 2H). LCMS (ESI): |
| | | | m/z 420.2 (M + H)⁺ |
| 4/13 | 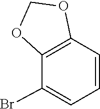 | 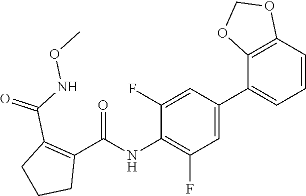 [SMILES: CONC(=O)C1=C(C(=O)Nc2c(F)cc(-c3cccc4c3OCO4)cc2F)CCC1] | ¹H-NMR (400 MHz, MeOD-d4) δ 7.50 (d, J = 9.2 Hz, 2H), 7.13 (d, J = 8.4 Hz, 1H), 6.95 (t, J = 7.8 Hz, 1H), 6.06 (s, 2H), 6.87 (d, J = 6.8 Hz, 1H), 3.75 (s, 3H), 2.92-2.87 (m, 2H), 2.82-2.77 (m, 2H), 2.08-1.98 (m, 2H). LCMS (ESI): m/z 417.1 (M + H)⁺ |
| 4/14 | 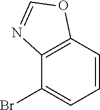 | 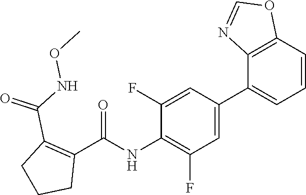 [SMILES: CONC(=O)C1=C(C(=O)Nc2c(F)cc(-c3cccc4ocnc34)cc2F)CCC1] | ¹H-NMR (400 MHz, MeOD-d4) δ 8.52 (s, 1H), 7.83-7.78 (m, 2H), 7.72-7.68 (m, 2H), 7.54 (t, J = 7.8 Hz, 1H), 3.75 (s, 3H), 2.92-2.87 (m, 2H), 2.84-2.78 (m, 2H), 2.06-1.97 (m, 2H). LCMS (ESI): m/z 414.1 (M + H)⁺ |
| 4/15 | 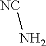 | 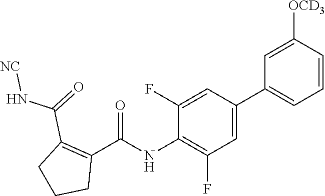 | ¹H-NMR (500 MHz, MeOH-d4) δ 7.40- 7.34 (m, 3H), 7.21 (d, J = 8.0 Hz, 1H), 7.17 (d, J = 2.0 Hz, 1H), 6.97 (dd, J = 2.3, 8.3 Hz, 1H), 2.99 (d, J = 7.3 Hz, 2H), 2.85 (d, J = 7.5 Hz, 2H), 2.12-2.04 (m, 2H). LCMS (ESI): m/z 401.2 (M + H)⁺ |
| 4/16 | 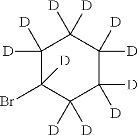 | 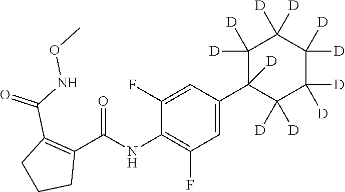 | |
| 4/17 | 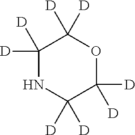 [SMILES: [2H]C1([2H])NC([2H])([2H])C([2H])([2H])OC1([2H])[2H]] | 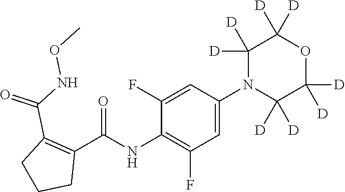 [SMILES: [2H]C1([2H])OC([2H])([2H])C([2H])([2H])N(c2cc(F)c(NC(=O)C3=C(C(=O)NOC)CCC3)c(F)c2)C1([2H])[2H]] | |
| 4/18 | 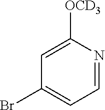 | 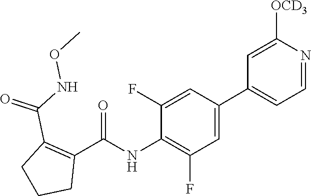 | |
| 4/19 | 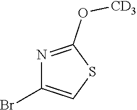 [SMILES: [2H]C([2H])([2H])Oc1nc(Br)cs1] | 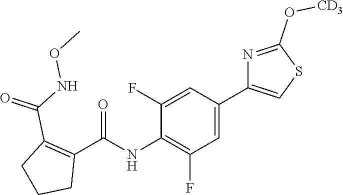 [SMILES: [2H]C([2H])([2H])Oc1nc(-c2cc(F)c(NC(=O)C3=C(C(=O)NOC)CCC3)c(F)c2)cs1] | |
| 4/20 | 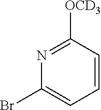 | 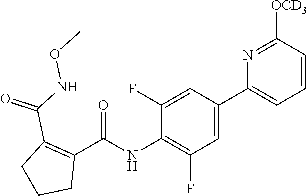 | |
| 4/21 | 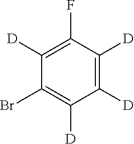 [SMILES: [2H]c1c([2H])c(F)c([2H])c(Br)c1[2H]] | 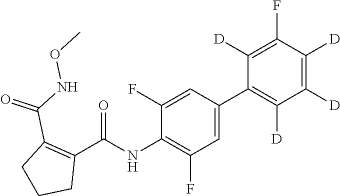 [SMILES: [2H]c1c([2H])c(F)c([2H])c(-c2cc(F)c(NC(=O)C3=C(C(=O)NOC)CCC3)c(F)c2)c1[2H]] | |
| 4/22 | 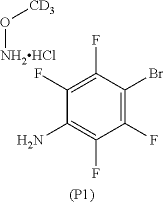 | 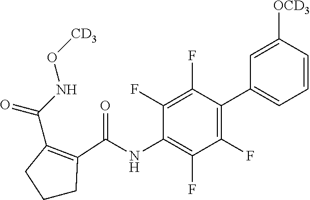 | ¹H-NMR (400 MHz, MeOD-d4) δ 7.42 (t, J = 8.0 Hz, 1H), 7.06-7.02 (m, 3H), 2.90 (t, J = 7.6 Hz, 2H), 2.80 (t, J = 7.2 Hz, 2H), 2.08-2.00 (m, 2H). LCMS (ESI): m/z 445.1 (M + H)⁺ |
| 4/23 | 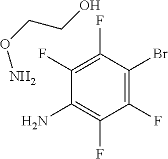 | 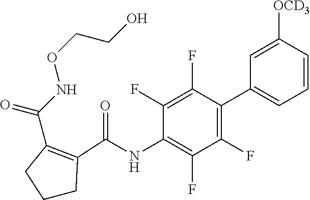 | ¹H-NMR (400 MHz, MeOD-d4) δ 7.42 (t, J = 8.0 Hz, 1H), 7.06-7.02 (m, 3H), 4.00 (t, J = 4.4 Hz, 2H), 3.72 (t, J = 4.8 Hz, 2H), 2.89 (t, J = 7.6 Hz, 2H), 2.81 (t, J = 7.6 Hz, 2H), 2.07-2.00 (m, 2H). LCMS (ESI): m/z 472.0 (M + H)⁺ |
| 4/24 | 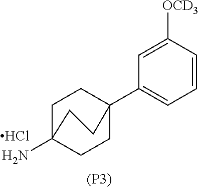 | 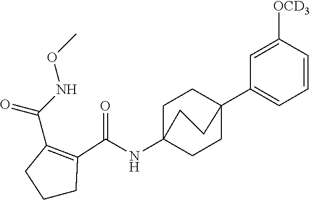 | ¹H NMR (400 MHz, MeOD-d4) δ 7.17 (t, J = 8.0 Hz, 1H), 6.89 (d, J = 8.0 Hz, 1H), 6.85-6.83 (m, 1H), 6.70 (dd, J = 2.8, 7.6 Hz, 1H), 3.74 (s, 3H), 2.75-2.63 (m, 4H), 2.10-1.87 (m, 14H). LCMS (ESI): m/z 402.3 (M + H)⁺ |
| 4/25 | 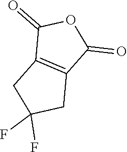 [SMILES: O=C1OC(=O)C2=C1CC(F)(F)C2] | 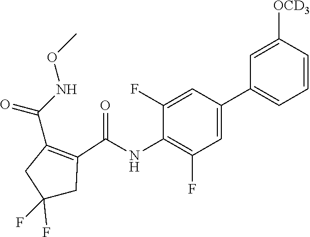 | ¹H-NMR (400 MHz, MeOD-d4) δ 7.39- 7.34 (m, 3H), 7.21- 7.16 (m, 2H), 6.97 (dd, J = 2.6, 8.2 Hz, 1H), 3.76 (s, 3H), 3.43-3.30 (m, 4H). LCMS (ESI): m/z 441.8 (M + H)⁺ |
| 4/26 | 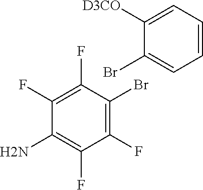 | 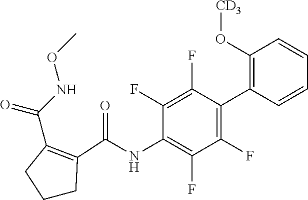 [SMILES: [2H]C([2H])([2H])Oc1ccccc1-c1c(F)c(F)c(NC(=O)C2=C(C(=O)NOC)CCC2)c(F)c1F] | ¹H-NMR (400 MHz, MeOD-d4) δ 7.50- 7.46 (m, 1H), 7.27 (d, J = 7.2 Hz, 1H), 7.14 (d, J = 8.0 Hz, 1H), 7.07 (t, J = 7.6 Hz, 1H), 3.76 (s, 3H), 2.92-2.88 (m, 2H), 2.82-2.77 (m, 2H), 2.08-2.01 (m, 2H). LCMS (ESI): m/z 442.4 (M + H)⁺ |
| 4/27 | 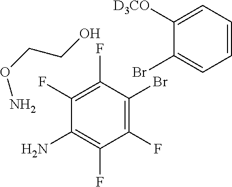 | 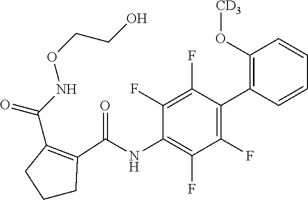 [SMILES: [2H]C([2H])([2H])Oc1ccccc1-c1c(F)c(F)c(NC(=O)C2=C(C(=O)NOCCO)CCC2)c(F)c1F] | ¹H-NMR (400 MHz, MeOD-d4) δ 7.51- 7.46 (m, 1H), 7.27 (dd, J = 1.2, 7.2 Hz, 1H), 7.14 (d, J = 8.8 Hz, 1H), 7.09-7.05 (m, 1H), 4.00 (t, J = 4.6 Hz, 2H), 3.75 (t, J = 4.6 Hz, 2H), 2.93-2.89 (m, 2H), 2.82-2.78 (m, 2H), 2.10-2.03 (m, 2H). LCMS (ESI): m/z 472.2 (M + H)⁺ |
| 4/28 | 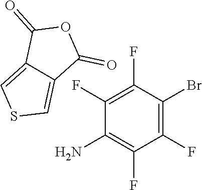 | 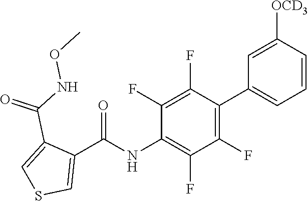 | ¹H-NMR (400 MHz, MeOD-d4) δ 8.33 (d, J = 2.8 Hz, 1H), 8.00 (d, J = 3.6 Hz, 1H), 7.45-7.41 (m, 1H), 7.07-7.04 (m, 3H), 3.82 (s, 3H). LCMS (ESI): m/z 458.0 (M + H)⁺ |
| 4/29 | 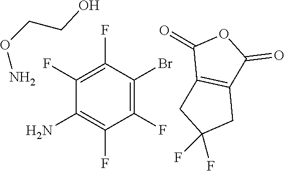 | 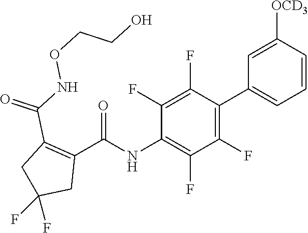 | ¹H-NMR (400 MHz, MeOD-d4) δ 7.42 (t, J = 8.0 Hz, 1H), 7.06-7.02 (m, 3H), 4.02 (t, J = 4.4 Hz, 2H), 3.76 (t, J = 4.6 Hz, 2H), 3.45-3.30 (m, 4H). LCMS (ESI): m/z 508.0 (M + H)⁺ |
| 4/30 | 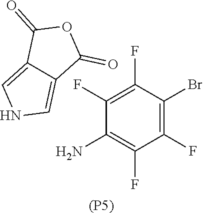 | 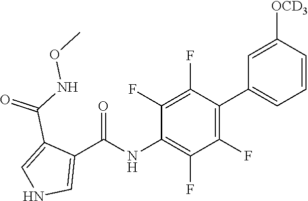 | ¹H-NMR (400 MHz, DMSO-d6) δ 12.28 (s, 1H), 12.01 (s, 1H), 11.98 (s, 1H), 7.72 (s, 1H), 7.56 (s, 1H), 7.48 (t, J = 7.8 Hz, 1H), 7.13- 7.08 (m, 3H), 3.73 (s, 3H). LCMS (ESI): m/z 441.1 (M + H)⁺ |
| 4/31 | 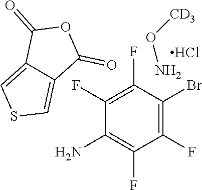 | 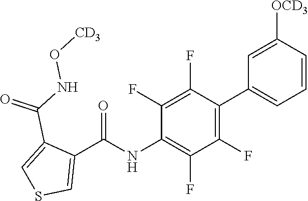 | ¹H-NMR (400 MHz, DMSO-d6) δ 11.70 (s, 1H), 11.01 (s, 1H), 8.33 (d, J = 3.2 Hz, 1H), 7.98 (d, J = 3.2 Hz, 1H), 7.48 (t, J = 7.8 Hz, 1H), 7.14-7.08 (m, 3H). LCMS (ESI): m/z 461.1 (M + H)⁺ |
| 4/32 | 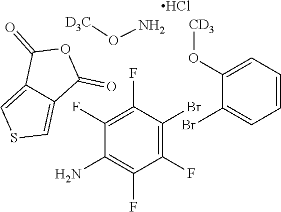 | 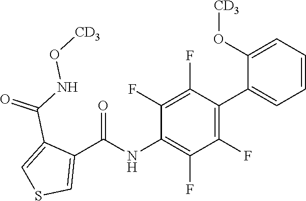 [SMILES: [2H]C([2H])([2H])ONC(=O)c1cscc1C(=O)Nc1c(F)c(F)c(-c2ccccc2OC([2H])([2H])[2H])c(F)c1F] | ¹H-NMR (400 MHz, MeOD-d4) δ 8.33 (d, J = 2.8 Hz, 1H), 8.01 (d, J = 3.2 Hz, 1H), 7.49 (dt, J = 1.4, 8.4 Hz, 1H), 7.29 (d, J = 7.6 Hz, 1H), 7.15 (d, J = 8.0 Hz, 1H), 7.08 (t, J = 7.4 Hz, 1H). LCMS (ESI): m/z 461.1 (M + H)⁺ |
| 4/33 | 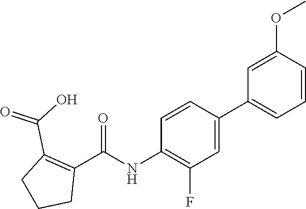 [SMILES: COc1cccc(-c2ccc(NC(=O)C3=C(C(=O)O)CCC3)c(F)c2)c1] | 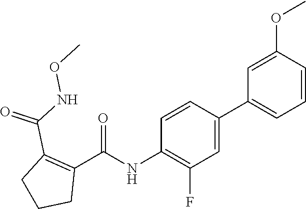 [SMILES: CONC(=O)C1=C(C(=O)Nc2ccc(-c3cccc(OC)c3)cc2F)CCC1] | ¹H-NMR (400 MHz, MeOD-d4) δ 8.14 (t, J = 8.2 Hz, 1H), 7.56-7.41 (m, 2H), 7.34 (t, J = 8.0 Hz, 1H), 7.19 (d, J = 7.6 Hz, 1H), 7.15 (d, J = 2.0 Hz, 1H), 6.91 (dd, J = 2.4, 8.0 Hz, 1H), 3.85 (s, 3H), 3.74 (s, 3H), 2.88- 2.79 (m, 4H), 1.95 (q, 7.6 Hz, 2H). |
| | | | LCMS (ESI): m/z |
| | | | 385.3 (M + H)⁺ |
| 4/34 | 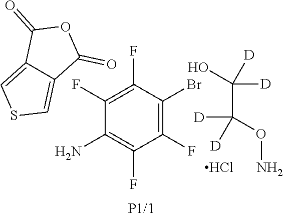 | 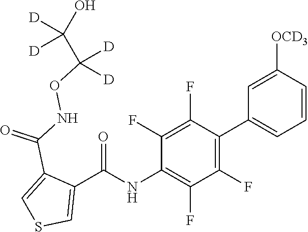 | |
| 4/35 | 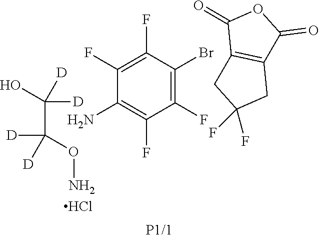 | 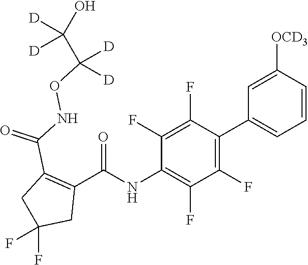 | |

### Example 5

### Step 1: Methyl 4-(((trifluoromethyl)sulfonyl)oxy)-2,5-dihydrofuran-3-carboxylate (5a)

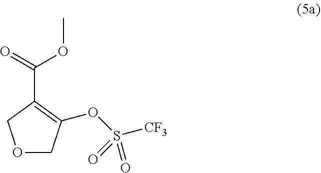 [SMILES: COC(=O)C1=C(OS(=O)(=O)C(F)(F)F)COC1]

A solution of methyl 4-hydroxy-2,5-dihydrofuran-3-carboxylate (33 g) in DCM (220 mL) was added DIPEA (88 g). Then Tf₂O (76.6 mL) was added to the mixture at 0° C. The mixture was stirred at rt overnight, concentrated and purified by FCC (PE:EA=20:1) to give compound 5a as an oil. LCMS (ESI): m/z=277.0 (M+H)⁺.

### Step 2: Dimethyl 2,5-dihydrofuran-3,4-dicarboxylate (5b)

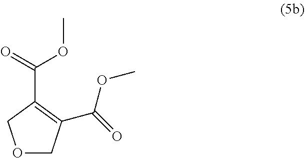

To a solution of compound 5a (30 g) in MeOH (225 mL) and DMF (75 mL) was added dppf (6.30 g) and Pd₂(dba)₃ (4.70 g). The mixture was stirred at 50° C. under CO overnight, concentrated and purified by FCC (PE:EA=10:1) to give compound 5b as a yellow oil. LCMS (ESI): m/z=187.3 (M+H)⁺.

### Step 3: 2,5-Dihydrofuran-3,4-dicarboxylic acid (5c)

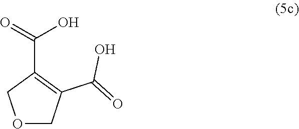 [SMILES: O=C(O)C1=C(C(=O)O)COC1]

To a solution of compound 5b (20 g) was added conc. HCl (150 mL) and HOAc (50 mL). The mixture was stirred at 100° C. for 2 h, cooled and concentrated to give compound 5c as a yellow solid. LCMS (ESI): m/z=159.3 (M+H)⁺.

### Step 4: 4,6-Dihydro-1H,3H-furo[3,4-c]furan-1,3-dione (5d)

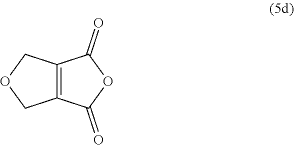

To a solution of compound 5c (400 mg) in toluene (5 mL) was added AcCl (385 mg). The mixture was stirred at 110° C. for 4 h and then concentrated under vacuum to afford compound 5d as a yellow solid, which was used for the next step without purification. LCMS (ESI): m/z=140.1 (M+H)⁺.

### Step 5: 4-((3,5-Difluoro-3′-(methoxy-d3)-[1,1′-biphenyl]-4-yl)carbamoyl)-2,5-dihydrofuran-3-carboxylic acid (5e)

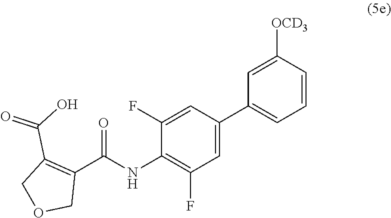

Compound 5d was reacted similar as described in Example 4, step 3, to yield compound 5e as a white solid. ¹H-NMR (500 MHz, DMSO-d6) δ 10.89 (br s, 1H), 7.58 (d, J=9.5 Hz, 2H), 7.39 (t, J=7.8 Hz, 1H), 7.33-7.28 (m, 2H), 6.99 (dd, J=2.3, 8.3 Hz, 1H), 4.97 (t, J=5.3 Hz, 2H), 4.89 (t, J=5.0 Hz, 2H), 3.43 (br s, 1H). LCMS (ESI): m/z 379.2 (M+H)⁺.

### Step 6: N³-(3,5-Difluoro-3′-(methoxy-d3)-[1,1′-biphenyl]-4-yl)-N⁴-methoxy-2,5-dihydrofuran-3,4-dicarboxamide (5)

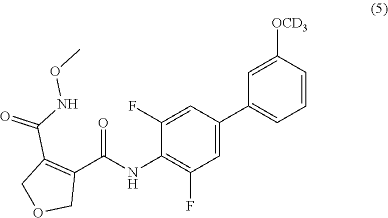

By reacting compound 5e similar as described in Example 4, step 4, the target compound 5 was obtained as a white solid. ¹H-NMR (400 MHz, DMSO-d6) δ 11.80 (s, 1H), 11.28 (br s, 1H), 7.60 (d, J=9.6 Hz, 2H), 7.40 (t, J=7.8 Hz, 1H), 7.34-7.29 (m, 2H), 6.99 (dd, J=2.0, 8.0 Hz, 1H), 4.96-4.92 (m, 4H), 3.68 (s, 3H). LCMS (ESI): m/z 408.2 (M+H)⁺.

### Example 5/1 to 5/16

The following Examples were prepared similar as described above using the appropriate building block(s) as shown below.

| # | building block(s) | structure | analytical data |
|---|---|---|---|
| 5/1 | 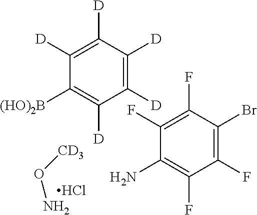 | 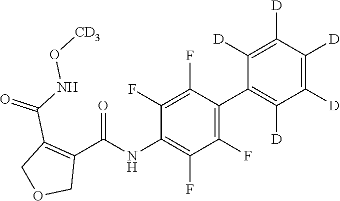 [SMILES: [2H]c1c([2H])c([2H])c(-c2c(F)c(F)c(NC(=O)C3=C(C(=O)NOC([2H])([2H])[2H])COC3)c(F)c2F)c([2H])c1[2H]] | ¹H-NMR (400 MHz, MeOD-d4) δ 5.07-5.04 (m, 3H), 5.01-4.99 (m, 2H). LCMS (ESI): m/z 419.1 (M + H)⁺ |
| 5/2 | 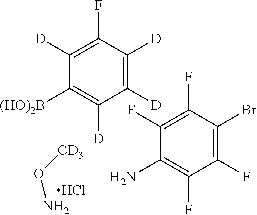 | 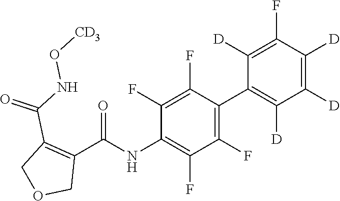 [SMILES: [2H]c1c([2H])c(F)c([2H])c(-c2c(F)c(F)c(NC(=O)C3=C(C(=O)NOC([2H])([2H])[2H])COC3)c(F)c2F)c1[2H]] | ¹H-NMR (400 MHz, MeOD-d4) δ 5.07-5.04 (m, 3H), 5.01-4.99 (m, 2H). LCMS (ESI): m/z 436.1 (M + H)⁺ |
| 5/3 | 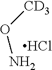 | 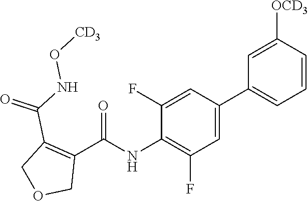 | ¹H-NMR (400 MHz, DMSO-d6) δ 11.80 (s, 1H), 11.30 (s, 1H), 7.60 (d, J = 9.2 Hz, 2H), 7.40 (t, J = 8.0 Hz, 1H), 7.34-7.30 (m, 2H), 6.99 (dd, J = 1.6, 8.0 Hz, 1H), 4.94 (s, 4H). LCMS (ESI): m/z 379.2 (M + H)⁺ |
| 5/4 | 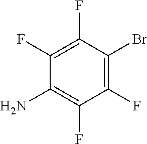 [SMILES: Nc1c(F)c(F)c(Br)c(F)c1F] | 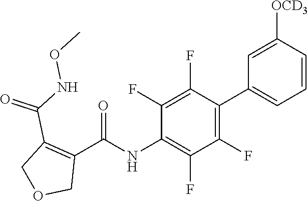 | ¹H-NMR (400 MHz, MeOD-d4) δ 7.42 (t, J = 8.0 Hz, 1H), 7.06-7.03 (m, 3H), 5.07-4.99 (m, 4H), 3.80 (s, 3H). LCMS (ESI): m/z 444.0 (M + H)⁺ |
| 5/5 | 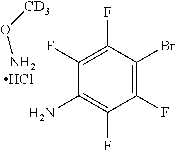 | 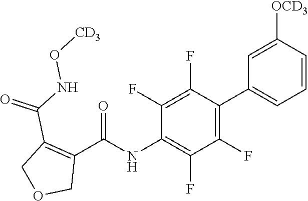 | ¹H-NMR (400 MHz, MeOD-d4) δ 7.42 (t, J = 8.2 Hz, 1H), 7.06- 7.03 (m, 3H), 5.07-4.99 (m, 4H). LCMS (ESI): m/z 447.0 (M + H)⁺ |
| 5/6 | 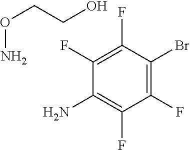 | 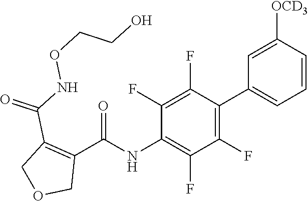 | ¹H-NMR (400 MHz, MeOD-d4) δ 7.42 (t, J = 8.0 Hz, 1H), 7.06- 7.03 (m, 3H), 5.08-5.01 (m, 4H), 4.04 (br s, 2H), 3.76 (t, J = 4.6 Hz, 2H). LCMS (ESI): m/z 474.0 (M + H)⁺ |
| 5/7 | 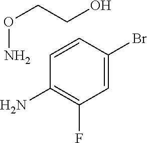 | 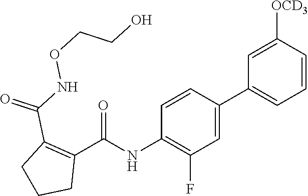 | ¹H-NMR (400 MHz, DMSO-d6) δ 11.50 (br s, 1H), 11.15 (br s, 1H), 8.13 (t, J = 7.6 Hz, 1H), 7.64 (dd, J = 2.0, 12.4 Hz, 1H), 7.53 (dd, J = 2.0, 8.4 Hz, 1H), 7.37 (t, J = 7.8 Hz, 1H), 7.28-7.23 (m, 2H), 6.94 (dd, J = 2.0, 8.0 Hz, 1H), 4.73 (br s, 1H), 3.88 (t, J = 5.0 Hz, 2H), 3.58 (t, J = 4.8 Hz, 2H), 2.81- 2.72 (m, 4H), 1.91-1.83 (m, 2H). LCMS (ESI): m/z 417.9 (M + H)⁺ |
| 5/8 | 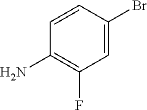 | 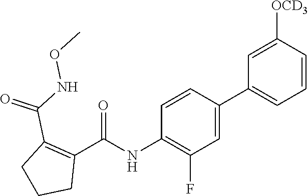 | ¹H-NMR (400 MHz, DMSO-d6) δ 11.52 (br s, 1H), 11.25 (br s, 1H), 8.16 (s, 1H), 7.64 (dd, J = 1.8, 12.6 Hz, 1H), 7.53 (dd, J = 1.4, 8.6 Hz, 1H), 7.37 (t, J = 7.8 Hz, 1H), 7.28-7.23 (m, 2H), 6.94 (dd, J = 2.0, 8.0 Hz, 1H), 3.66 (s, 3H), 2.80-2.70 (m, 4H), 1.91-1.83 (m, 2H). LCMS (ESI): m/z 388.0 (M + H)⁺ |
| 5/9 | 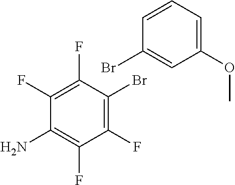 | 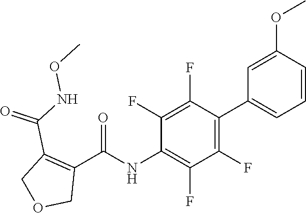 [SMILES: CONC(=O)C1=C(C(=O)Nc2c(F)c(F)c(-c3cccc(OC)c3)c(F)c2F)COC1] | ¹H-NMR (400 MHz, DMSO-d6) δ 11.81 (br s, 2H), 7.47 (t, J = 8.2 Hz, 1H), 7.13-7.09 (m, 3H), 4.96-4.93 (m, 4H), 3.80 (s, 3H), 3.70 (s, 3H). LCMS (ESI): m/z 441.1 (M + H)⁺ |
| 5/10 | 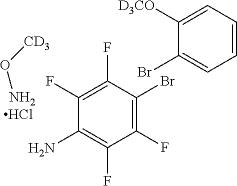 | 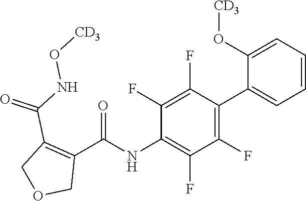 [SMILES: [2H]C([2H])([2H])ONC(=O)C1=C(C(=O)Nc2c(F)c(F)c(-c3ccccc3OC([2H])([2H])[2H])c(F)c2F)COC1] | ¹H-NMR (400 MHz, DMSO-d6) δ 11.86 (br s, 2H), 7.53 (dt, J = 1.8, 7.2 Hz, 1H), 7.39 (d, J = 6.0 Hz, 1H), 7.22 (d, J = 8.4 Hz, 1H), 7.10 (t, J = 7.4 Hz, 1H), 4.96-4.91 (m, 4H). LCMS (ESI): m/z 447.3 (M + H)⁺ |
| 5/11 | 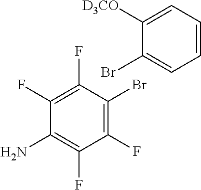 | 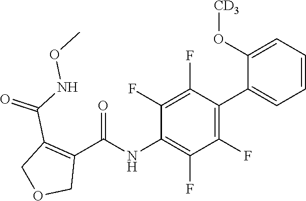 [SMILES: [2H]C([2H])([2H])Oc1ccccc1-c1c(F)c(F)c(NC(=O)C2=C(C(=O)NOC)COC2)c(F)c1F] | ¹H-NMR (400 MHz, DMSO-d6) δ 11.86 (br s, 2H), 7.53 (dt, J = 1.8, 7.2 Hz, 1H), 7.39 (d, J = 7.2 Hz, 1H), 7.22 (d, J = 8.0 Hz, 1H), 7.11 (t, J = 7.4 Hz, 1H), 4.96-4.91 (m, 4H), 3.69 (s, 3H). LCMS (ESI): m/z 444.2 (M + H)⁺ |
| 5/12 | 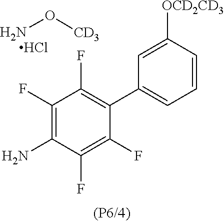 | 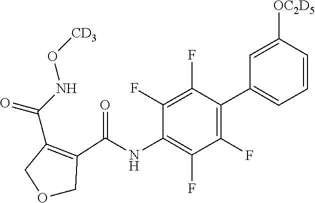 | ¹H-NMR (400 MHz, CD₃OD) δ 7.41 (t, J = 7.8 Hz, 1H), 7.04-7.01 (m, 3H), 5.07-4.98 (m, 4H). LCMS (ESI): m/z 463.3 (M + H)⁺ |
| 5/13 | 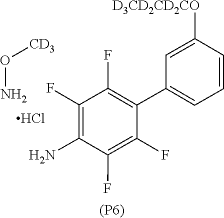 | 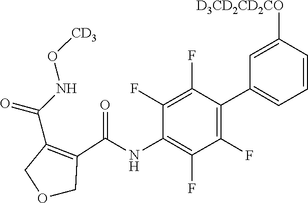 | ¹H-NMR (400 MHz, DMSO-d6) δ 11.79 (br s, 2H), 7.45 (t, J = 7.8 Hz, 1H), 7.11-7.07 (m, 3H), 4.95 (s, 4H). LCMS (ESI): m/z 479.1 (M + H)⁺ |
| 5/14 | 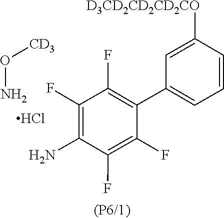 | 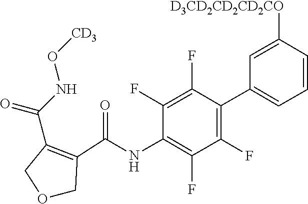 | ¹H-NMR (400 MHz, DMSO-d6) δ 11.81 (br s, 2H), 7.45 (t, J = 8.0 Hz, 1H), 7.12-7.07 (m, 3H), 4.90 (s, 4H). LCMS (ESI): m/z 495.3 (M + H)⁺ |
| 5/15 | 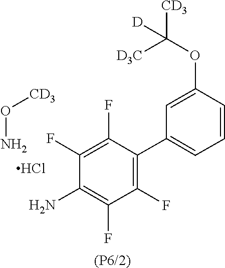 | 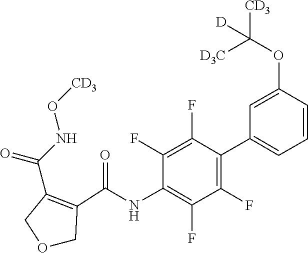 [SMILES: [2H]C([2H])([2H])ONC(=O)C1=C(C(=O)Nc2c(F)c(F)c(-c3cccc(OC([2H])(C([2H])([2H])[2H])C([2H])([2H])[2H])c3)c(F)c2F)COC1] | ¹H-NMR (400 MHz, DMSO-d6) δ 11.79 (br s, 2H), 7.44 (t, J = 8.0 Hz, 1H), 7.10-7.04 (m, 3H), 4.96-4.92 (m, 4H). LCMS (ESI): m/z 479.2 (M + H)⁺ |
| 5/16 | 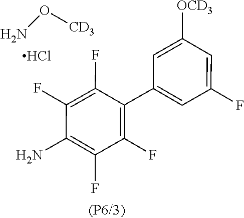 | 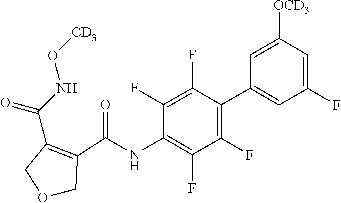 | ¹H-NMR (400 MHz, DMSO-d6) δ 11.82 (br s, 1H), 11.79 (br s, 1H), 7.05-7.01 (m, 3H), 4.94 (s, 4H). LCMS (ESI): m/z 465.3 (M + H)⁺ |

### Example 6

### Step 1: 4-((3,5-Difluoro-3′-(methoxy-d3)-[1,1′-biphenyl]-4-yl)carbamoyl)-2,5-dihydrothiophene-3-carboxylic acid (6a)

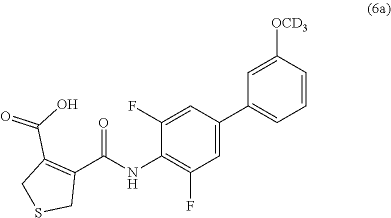

By reacting 4,6-dihydro-1H,3H-thieno[3,4-c]furan-1,3-dione (synthesis and coupling described in Bioorg. Med. Chem. Lett. 2005; 15:4854) similar as described above, the target molecule 6a was obtained as a white solid. ¹H-NMR (400 MHz, DMSO-d6) δ 13.01 (br s, 1H), 10.20 (s, 1H), 7.54 (d, J=9.2 Hz, 2H), 7.39 (t, J=7.8 Hz, 111), 7.32-2.28 (m, 2H), 6.99 (dd, J=2.4, 8.0 Hz, 1H), 4.15-4.11 (m, 2H), 4.03-4.00 (m, 2H). LCMS (ESI): m/z 395.2 (M+H)⁺.

### Step 2: N³-(3,5-Difluoro-3′-(methoxy-d3)-[1,1′-biphenyl]-4-yl)-N⁴-methoxy-2,5-dihydrothiophene-3,4-dicarboxamide (6)

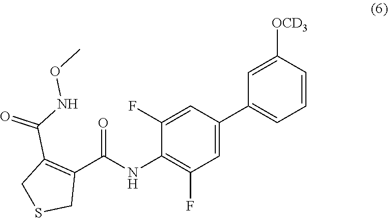

By reacting compound 6a similar as described above, the target compound 6 was obtained as a white solid. ¹H-NMR (500 MHz, MeOD-d4) δ 7.38-7.32 (m, 3H), 7.20 (d, J=7.5 Hz, 1H), 7.16 (s, 1H), 6.96 (dd, J=2.5, 8.0 Hz, 1H), 4.20-4.11 (m, 4H), 3.71 (s, 3H). LCMS (ESI): m/z 424.1 (M+H)⁺.

### Example 6/1 to 6/3

The following Examples were prepared similar as described for Example 6 above using the appropriate building block(s) as shown below.

| # | building block(s) | structure | analytical data |
|---|---|---|---|
| 6/1 | 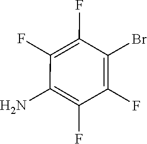 [SMILES: Nc1c(F)c(F)c(Br)c(F)c1F] | 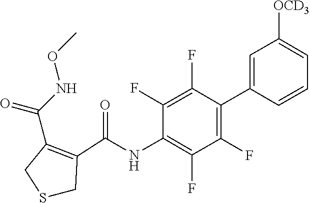 | ¹H-NMR (400 MHz, MeOD-d4) δ 7.42 (t, J = 8.0 Hz, 1H), 7.06-7.02 (m, 3H), 4.21-4.18 (m, 2H), 4.09-4.06 (m, 2H), 3.74 (s, 3H). LCMS (ESI): m/z 460.0 (M + H)⁺ |
| 6/2 | 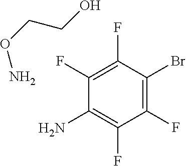 | 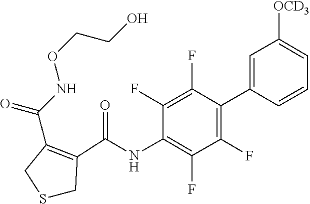 | ¹H-NMR (400 MHz, MeOD-d4) δ 7.42 (t, J = 7.8 Hz, 1H), 7.06-7.02 (m, 3H), 4.21 (t, J = 4.6 Hz, 2H), 4.09 (t, J = 4.6 Hz, 2H), 3.88 (t, J = 4.4 Hz, 2H), 3.73 (t, J = 4.6 Hz, 2H). LCMS (ESI): m/z 490.0 (M + H)⁺ |
| 6/3 | 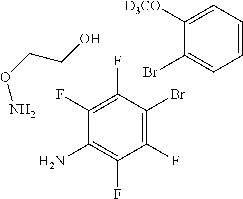 | 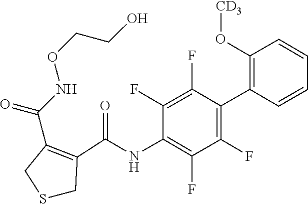 [SMILES: [2H]C([2H])([2H])Oc1ccccc1-c1c(F)c(F)c(NC(=O)C2=C(C(=O)NOCCO)CSC2)c(F)c1F] | ¹H-NMR (400 MHz, MeOD-d4) δ 7.51-7.46 (m, 1H), 7.28-7.26 (m, 1H), 4.14 (d, J = 8.0 Hz, 1H), 7.09-7.04 (m, 1H), 4.21 (t, J = 4.2 Hz, 2H), 4.09 (t, J = 4.4 Hz, 2H), 3.99 (t, J = 3.8 Hz, 2H), 3.73 (t, J = 4.2 Hz, 2H). LCMS (ESI): m/z 490.0 (M + H)⁺ |

### Example 7

### N¹—(N-Cyanosulfamoyl)-N²-(3,5-difluoro-3′-(methoxy-d3)-[1,1′-biphenyl]-4-yl)cyclopent-1-ene-1,2-dicarboxamide (7)

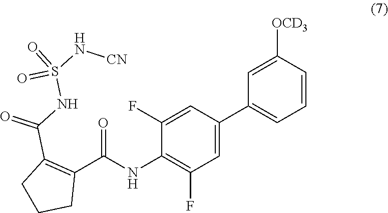

By reacting compound 2/1 with cyanic bromide in a solution of KOH in DMF/H₂O, the target molecule 7 was obtained. LCMS (ESI): m/z 480.1 (M+H)⁺.

### Example 8 (Inverse Coupling Procedure)

### Step 1: 3-Fluoro-5-(3-(methoxy-d3)phenyl)pyridin-2-amine (8a)

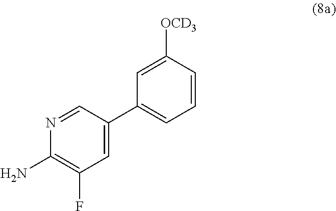

To a solution of 5-bromo-3-fluoropyridin-2-amine (400 mg) in 1,4-dioxane (5 mL) and H₂O (0.5 mL) was added (3-(methoxy-d3)phenyl)boronic acid (389 mg), Cs₂CO₃ (2.4 g) and Pd(dppf)Cl₂ (40 mg). The mixture was stirred at 90° C. under N₂ for 2 h, cooled to rt, concentrated and purified by FCC (PE:EA=10:1) to give compound 8a as a yellow solid. LCMS (ESI): m/z 222.0 (M+H)⁺.

### Step 2: N¹-(3-Fluoro-5-(3-(methoxy-d₃)phenyl)pyridin-2-yl)-N²-methoxycyclopent-1-ene-1,2-dicarboxamide (8)

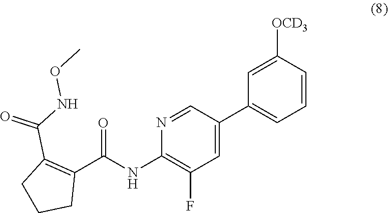

By reacting compound 8a as described in Example 4, step 3 and step 4, target molecule 8 was obtained as a white solid. ¹H-NMR (500 MHz, DMSO-d6) δ 11.39 (br s, 1H), 11.04 (br s, 1H), 8.62 (s, 1H), 8.15 (d, J=11.0 Hz, 1H), 7.44-7.33 (m, 3H), 7.00 (dd, J=2.0, 8.0 Hz, 1H), 3.63 (s, 3H), 2.79 (t, J=6.8 Hz, 2H), 2.69 (t, J=6.8 Hz, 2H), 1.93-1.87 (m, 2H). LCMS (ESI): m/z 389.3 (M+H)⁺.

### Example 8/1 to 8/3

The following Examples were prepared similar as described for Example 8 above using the appropriate building block as shown below.

| # | building block | structure | analytical data |
|---|---|---|---|
| 8/1 | 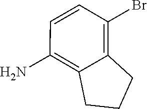 [SMILES: Nc1ccc(Br)c2c1CCC2] | 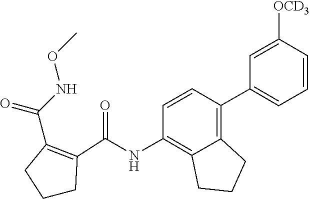 | ¹H-NMR (500 MHz, MeOD-d4) δ 7.63 (d, J = 7.0 Hz, 1H), 7.31 (t, J = 8.0 Hz, 1H), 7.16 (d, J = 8.0 Hz, 1H), 6.98 (d, J = 7.5 Hz, 1H), 6.94 (s, 1H), 6.87 (dd, J = 1.8, 8.3 Hz, 1H), 3.75 (s, 3H), 3.00-2.97 (m, 4H), 2.89 (t, J = 7.5 Hz, 2H), 2.79 (t, J = 7.5 Hz, 2H), 2.10-1.96 (m, 4H). LCMS (ESI): m/z 410.2 (M + H)⁺ |
| 8/2 | 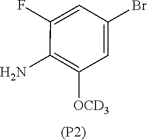 | 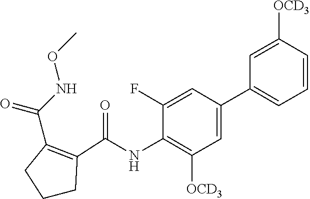 | ¹H-NMR (400 MHz, MeOD-d4) δ 7.36 (d, J = 8.0 Hz, 1H), 7.20 (d, J = 8.0 Hz, 1H), 7.15 (t, J = 2.0 Hz, 1H), 7.07-7.03 (m, 2H), 6.95-6.93 (m, 1H), 3.74 (s, 3H), 2.90 (t, J = 7.2 Hz, 2H), 2.80 (t, J = 7.2 Hz, 2H), 2.04-1.97 (m, 2H). LCMS (ESI): m/z 421.3 (M + H)⁺ |
| 8/3 | 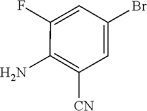 [SMILES: N#Cc1cc(Br)cc(F)c1N] | 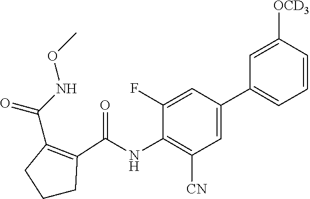 | ¹H-NMR (500 MHz, DMSO-d6) δ 11.45 (s, 1H), 11.03 (s, 1H), 8.13-8.07 (m, 2H), 7.43-7.36 (m, 3H), 7.01 (d, J = 6.5 Hz, 1H), 3.65 (s, 3H), 2.83-2.70 (m, 4H), 1.96-1.90 (m, 2H). LCMS (ESI): m/z 413.2 (M + H)⁺ |

### Example 9

### Step 1: Methyl 3-(chlorocarbonyl)thiophene-2-carboxylate (9a)

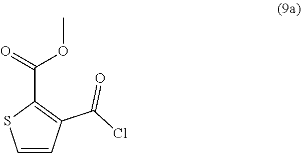

To a solution of 2-(methoxycarbonyl)thiophene-3-carboxylic acid (200 mg) in dry DCM (8 mL) was added SOCl₂ (152 mg). The mixture was stirred at rt for 2 h and concentrated to give compound 9a as a yellow solid, which was used to next step without further purification.

### Step 2: Methyl 3-((3,5-difluoro-3′-(methoxy-d3)-[1,1′-biphenyl]-4-yl)carbamoyl)thiophene-2-carboxylate (9b)

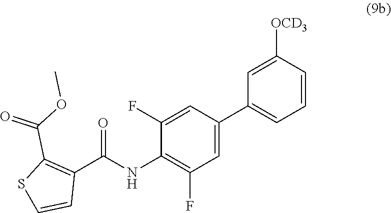

To a solution of compound 4b (200 mg) in dry THE (2 mL) was added NaH (60%, 134 mg) at 0° C. The reaction mixture was stirred for 1 h and a solution of crude intermediate 9a (240 mg) in dry THF (1 mL) was added dropwise at a 0° C. After addition, the mixture was stirred for 30 min at this temperature, quenched with saturated aq. NH₄Cl and extracted with EA (3×20 mL). The combined organic layer was dried over Na₂SO₄, filtered, concentrated and purified by FCC (PE:EA=1:1) to give compound 9b as a red solid. LCMS (ESI): m/z 407.1 (M+H)⁺.

### Step 3: 3-((3,5-Difluoro-3′-(methoxy-d3)-[1,1′-biphenyl]-4-yl)carbamoyl)thiophene-2-carboxylic acid (9c)

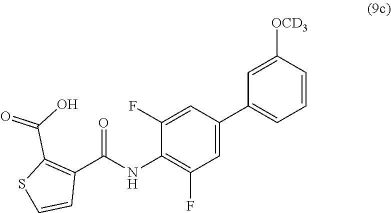

To a solution of compound 9b (200 mg) in MeOH (4 mL) and THE (1.5 mL) was added 2N NaOH (1.0 mL) at 0° C. Then the mixture was stirred at rt for 5 h, adjusted to pH=5-6 by 2N HCl and concentrated. The residue was purified by reversed-phase flash chromatography (C18) (0.1% NH₄HCO₃ in water, 10 to 100% MeCN) to give compound 9c as a white solid. ¹H-NMR (400 MHz, CD₃OD) δ 7.70 (d, J=5.2 Hz, 1H), 7.47 (d, J=5.2 Hz, 1H), 7.40-7.32 (m, 3H), 7.22-7.17 (m, 2H), 6.96 (dd, J=2.0, 8.4 Hz, 1H). LCMS (ESI): m/z 393.1 (M+H)⁺.

### Step 4: N3-(3,5-Difluoro-3′-(methoxy-d3)-[1,1′-biphenyl]-4-yl)-N2-methoxythiophene-2,3-di-carboxamide (9)

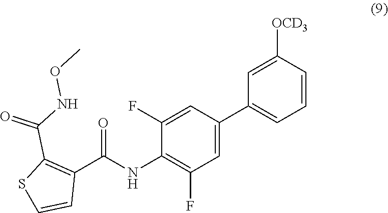

A solution of 9c (90 mg) in MeCN (5 mL) was added O-methylhydroxylamine hydrochloride (28 mg), TCFH (162 mg) and 1-methylimidazole (57 mg). The mixture was stirred at rt for 4 h, concentrated and purified by reversed-phase flash chromatography (C18) (0.1% TFA in water, 10 to 100% MeCN) to afford target compound 9 as a white solid. ¹H-NMR (400 MHz, CD₃OD) δ 7.79 (d, J=5.2 Hz, 1H), 7.67 (d, J=5.2 Hz, 1H), 7.42-7.36 (m, 3H), 7.24-7.18 (m, 2H), 6.99-6.96 (m, 1H), 3.82 (s, 3H). LCMS (ESI): m/z 422.1 (M+H)⁺.

### Example 9/1 to 9/11

The following Examples were prepared similar as described for Example 9 or other examples above using the appropriate building block(s) as shown below.

| # | building block(s) | structure | analytical data |
|---|---|---|---|
| 9/1 | 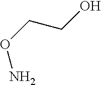 | 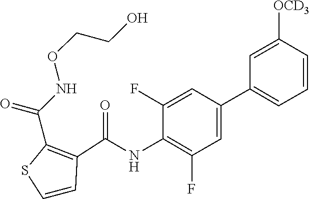 | ¹H-NMR (400 MHz, MeOD-d4) δ 7.80 (d, J = 5.2 Hz, 1H), 7.68 (d, J = 5.6 Hz, 1H), 7.40-7.36 (m, 3H), 7.23-7.18 (m, 2H), 6.99-6.96 (m, 1H), 4.05 (t, J = 4.6 Hz, 2H), 3.78-3.75 (m, 2H). LCMS (ESI): m/z 452.0 (M + H)⁺ |
| 9/2 | 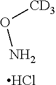 | 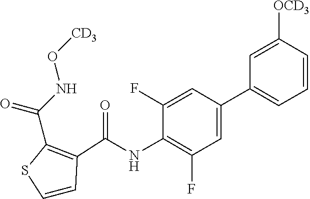 | ¹H-NMR (400 MHz, DMSO-d6) δ 12.21 (br s, 1H), 10.61 (s, 1H), 7.91 (d, J = 5.2 Hz, 1H), 7.68-7.61 (m, 3H), 7.41 (t, J = 8.0 Hz, 1H), 7.35-7.32 (m, 2H), 7.00 (dd, J = 1.8, 8.2 Hz, 1H). LCMS (ESI): m/z 425.1 (M + H)⁺ |
| 9/3 | 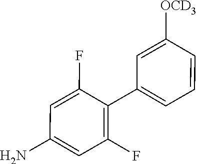 | 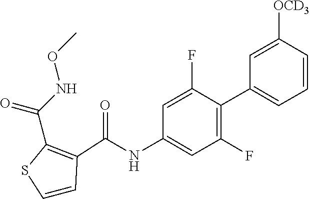 | LCMS (ESI): m/z 422.1 (M + H)⁺ |
| 9/4 | 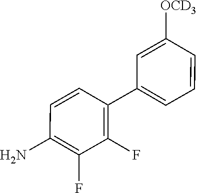 | 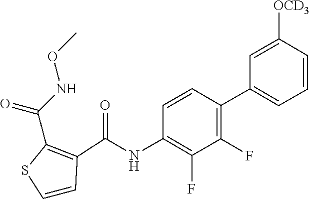 | ¹H-NMR (400 MHz, MeOD-d4) δ 7.88 (t, J = 7.2 Hz, 1H), 7.74 (d, J = 5.2 Hz, 1H), 7.67 (d, J = 4.8 Hz, 1H), 7.38 (t, J = 8.0 Hz, 1H), 7.33-7.28 (m, 1H), 7.14-7.09 (m, 2H), 6.97 (dd, J = 1.8, 8.2 Hz, 1H), 3.83 (m, 3H). LCMS (ESI): m/z 422.0 (M + H)⁺ |
| 9/5 | 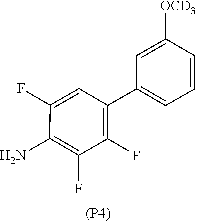 | 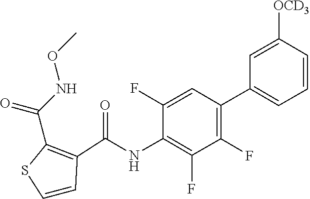 | ¹H-NMR (400 MHz, MeOD-d4) δ 7.73 (d, J = 5.2 Hz, 1H), 7.67 (d, J = 5.2 Hz, 1H), 7.41 (t, J = 8.0 Hz, 1H), 7.27-7.22 (m, 1H), 7.16-7.12 (m, 2H), 7.03-7.00 (m, 1H), 3.81 (m, 3H). LCMS (ESI): m/z 440.1 (M + H)⁺ |
| 9/6 | 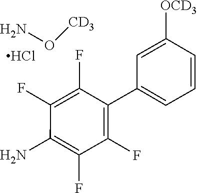 | 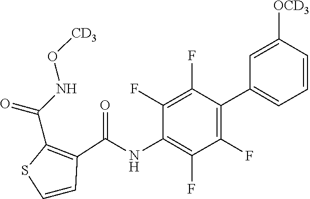 | ¹H-NMR (400 MHz, MeOD-d4) δ 7.79 (d, J = 5.6 Hz, 1H), 7.68 (d, J = 5.2 Hz, 1H), 7.45-7.41 (m, 1H), 7.07-7.04 (m, 3H). LCMS (ESI): m/z 461.1 (M + H)⁺ |
| 9/7 | 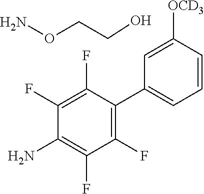 | 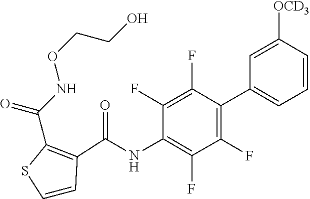 | ¹H-NMR (400 MHz, MeOD-d4) δ 7.77 (d, J = 5.2 Hz, 1H), 7.68 (d, J = 5.2 Hz, 1H), 7.43 (t, J = 8.0 Hz, 1H), 7.07-7.05 (m, 3H), 4.06 (t, J = 4.6 Hz, 2H), 3.78 (t, J = 4.8 Hz, 2H). LCMS (ESI): m/z 488.0 (M + H)⁺ |
| 9/8 | 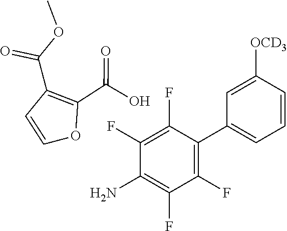 | 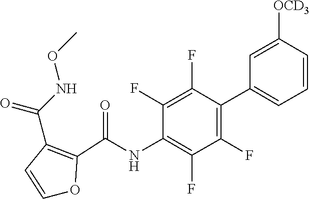 | ¹H-NMR (400 MHz, DMSO-d6) δ 12.37 (s, 1H), 12.29 (s, 1H), 8.14 (d, J = 2.0 Hz, 1H), 7.48 (t, J = 8.0 Hz, 1H), 7.15-7.09 (m, 4H), 3.76 (s, 3H). LCMS (ESI): m/z 442.1 (M + H)⁺ |
| 9/9 | 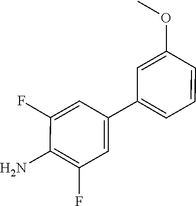 | 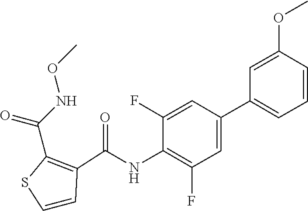 [SMILES: CONC(=O)c1sccc1C(=O)Nc1c(F)cc(-c2cccc(OC)c2)cc1F] | ¹H-NMR (400 MHz, DMSO-d6) δ 12.20 (s, 1H), 10.61 (s, 1H), 7.91 (d, J = 5.2 Hz, 1H), 7.67-7.61 (m, 3H), 7.43-7.31 (m, 3H), 7.01 (dd, J = 1.8, 8.2 Hz, 1H), 3.85 (s, 3H), 3.70 (s, 3H). LCMS (ESI): m/z 419.1 (M + H)⁺ |
| 9/10 | 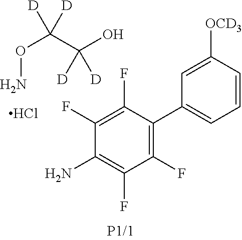 | 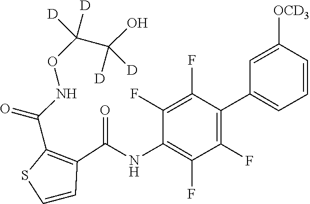 | ¹H-NMR (500 MHz, DMSO-d6) δ 12.04 (s, 1H), 10.99 (s, 1H), 7.91 (d, J = 5.0 Hz, 1H), 7.65 (d, J = 3.0 Hz, 1H), 7.48 (t, J = 8.0 Hz, 1H), 7.15-7.09 (m, 3H), 4.28 (br s, 1H). LCMS (ESI): m/z 492.2 (M + H)⁺ |
| 9/11 | 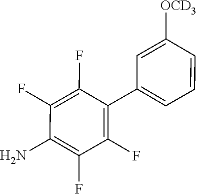 | 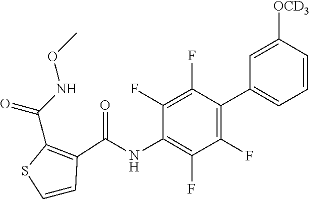 | ¹H-NMR (500 MHz, DMSO-d6) δ 12.03 (s, 1H), 11.00 (s, 1H), 7.90 (d, J = 5.0 Hz, 1H), 7.64 (br s, 1H), 7.48 (t, J = 8.0 Hz, 1H), 7.15-7.09 (m, 3H), 3.71 (s, 3H). LCMS (ESI): m/z 58.2 (M + H)⁺ |

### Example 10

### Step 1: Methyl 3-(methoxycarbamoyl)thiophene-2-carboxylate (10a)

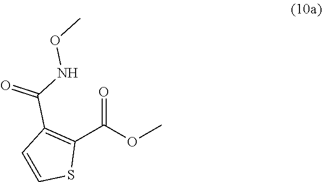

A solution of 2-(methoxycarbonyl)thiophene-3-carboxylic acid (200 mg) in MeCN (5 mL) was added O-methylhydroxylamine hydrochloride (178 mgl), TCFH (361 mg) and 1-methylimidazole (264 mg). The mixture was stirred at rt for 4 h, concentrated and purified by reversed-phase flash chromatography (C18) (0.1% TFA in water, 10 to 100% MeCN) to give compound 10a as a white solid. LCMS (ESI): m/z 216.1 (M+H)⁺.

### Step 2: 3-(Methoxycarbamoyl)thiophene-2-carboxylic acid (10b)

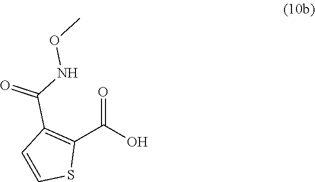

To a solution of compound 10a (100 mg) in MeOH (1 mL) and THE (3 mL) was added 2N NaOH (2 mL) at 0° C. The mixture was stirred at 0° C. for 1 h, adjusted to pH 5-6 by addition of 2N HCl, concentrated and purified by reversed-phase flash chromatography (C18) (0.1% TFA in water, 10 to 100% MeCN) to give compound 10b as a white solid. LCMS (ESI): m/z 202.1 (M+H)⁺.

### Step 3: N²-(3,5-Difluoro-3′-(methoxy-d3)-[1,1′-biphenyl]-4-yl)-N³-methoxythiophene-2,3-dicarboxamide (10)

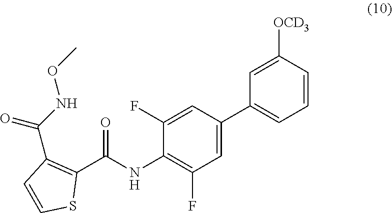

To a solution of compound 10b (50 mg) in dry DCM (5 mL) was added SOCl₂ (59 mg) at 0° C. The mixture was stirred at 0° C. for 2 h and concentrated to afford the crude acid chloride intermediate. To a solution of compound 4b (59 mg) in dry THE (5 mL) was added NaH (99 mg, 60% wt) at 0° C., stirred at 0° C. for 10 min and then the acid chloride intermediate was added at 0° C. The mixture was stirred at 0° C. for 2 h, quenched with saturated aq. NH₄Cl and extracted with EA (3×20 mL). The combined organic layer was dried over Na₂SO₄, filtered, concentrated and purified by reversed-phase flash chromatography (C18) (0.1% TFA in water, 10 to 100% MeCN) to give compound 10 as a white solid. ¹H-NMR (400 MHz, DMSO-d6) δ 12.28 (s, 1H), 11.94 (s, 1H), 7.98 (d, J=5.2 Hz, 1H), 7.62 (d, J=9.2 Hz, 2H), 7.49 (d, J=5.2 Hz, 1H), 7.43-7.31 (m, 3H), 7.00 (dd, J=1.6, 8.0 Hz, 1H), 3.77 (s, 3H). LCMS (ESI): m/z 422.1 (M+H)⁺.

### Example 10/1

The following Example was prepared similar as described for Example 10 above using the appropriate building blocks as shown below.

| # | building blocks | structure | analytical data |
|---|---|---|---|
| 10/1 | 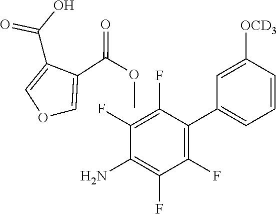 | 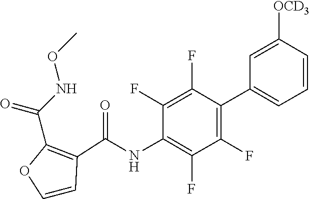 | ¹H-NMR (400 MHz, DMSO-d6) δ 12.63 (br s, 2H), 8.07 (d, J = 1.6 Hz, 1H), 7.48 (t, J = 8.0 Hz, 1H), 7.16-7.08 (m, 4H), 3.78 (s, 3H). LCMS (ESI): m/z 442.1 (M + H)⁺ |

### Example 13

### Step 1: Di-tert-butyl (3,5-difluoro-3′-(methoxy-d3)-[1,1′-biphenyl]-4-yl)iminodicarbonate (13a)

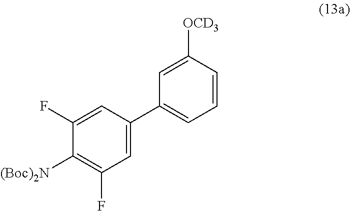

To a solution of intermediate 4b (400 mg) in DMF (5 mL) was added di-tert-butyl pyrocarbonate (732 mg) and DIPEA (434 mg). The mixture was stirred at rt for 8 h, concentrated and purified by preparative HPLC to afford compound 13a as a yellow solid. LCMS (ESI): m/z 384.0 (M+H)⁺.

### Step 2: tert-Butyl (3,5-difluoro-3′-(methoxy-d3)-[1,1′-biphenyl]-4-yl)carbamate (13b)

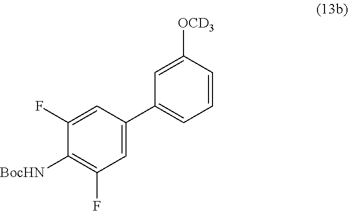

To a solution of compound 13a (500 mg) in THE (5 mL) was added 2M KOH (3 mL) at 0° C. The mixture was stirred at rt for 8 h, concentrated and purified by FCC (PE:EA=5:1) to afford compound 13b as a yellow solid. LCMS (ESI): m/z 284.0 (M+H-tert-butyl)⁺.

### Step 3: tert-Butyl (3,5-difluoro-3′-(methoxy-d3)-[1,1′-biphenyl]-4-yl)(methyl)carbamate (13c)

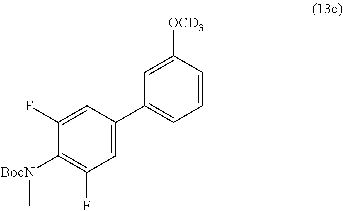

To a solution of compound 13b (200 mg) in dry THE (5 mL) was added NaH (118 mg, 60% wt) at 0° C. The mixture was stirred at 0° C. for 1 h, then CH₃I (101 mg) was added and the mixture was stirred overnight, concentrated and purified by preparative HPLC to afford compound 13c as a white solid. LCMS (ESI): m/z 287.1 (M+H-tert-butyl)⁺.

### Step 4: 3,5-Difluoro-3′-(methoxy-d3)-N-methyl-[1,1′-biphenyl]-4-amine (13d)

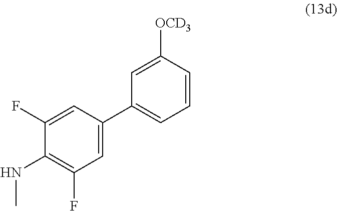

To a solution of compound 13c (190 mg) in 1,4 dioxane (5 mL) was added HCl in dioxane (4M, 5 mL) The mixture was stirred at t for 1 h and concentrated under vacuum to afford compound 13d as a white solid, which was used for the next step without further purification. LCMS (ESI): m/z 253.1 (M+H)⁺.

### Step 5: 2-((3,5-Difluoro-3′-(methoxy-d3)-[1,1′-biphenyl]-4-yl)(methyl)carbamoyl)cyclopent-1-ene-1-carboxylic acid (13e)

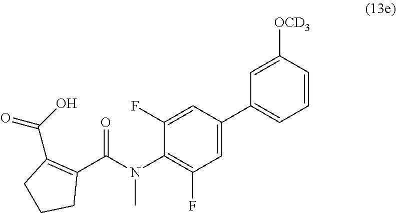

By coupling compound 13d with 1-cyclopentene-1,2-dicarboxylic anhydride similar as described above, the compound 13e was prepared. LCMS (ESI): m/z 391.1 (M+H)⁺.

### Step 6: N1-(3,5-Difluoro-3′-(methoxy-d3)-[1,1′-biphenyl]-4-yl)-N2-methoxy-N1-methylcyclopent-1-ene-1,2-dicarboxamide (13)

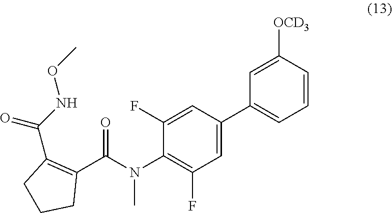

By coupling compound 13e with O-methylhydroxylamine hydrochloride similar as described above in Example 9, step 4, the target compound 13 was obtained as a white solid after purification via preparative HPLC. ¹H-NMR (400 MHz, MeOD-d4, mixture of E/Z-isomer) δ 7.40-7.36 (m, 3H), 7.21 (d, J=8.4 Hz, 1H), 7.17 (d, J=2.0 Hz, 1H), 6.98 (dd, J=2.0, 8.4 Hz, 1H), 3.74/3.67 (m, 3H), 3.31-3.30 (m, 3H), 2.83-2.37 (m, 4H), 2.19-2.09 (m, 0.61H), 1.86-1.77 (m, 1.36H). LCMS (ESI): m/z 420.3 (M+H)⁺.

### Example 14

### Step 1: Methyl (3,5-difluoro-3′-(methoxy-d3)-[1,1′-biphenyl]-4-yl)glycinate (14a)

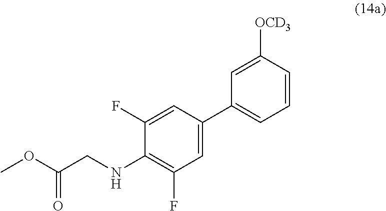

To a solution of intermediate 4b (500 mg) in toluene (10 mL) was added methyl 2-bromoacetate (479 mg) and K₂CO₃ (580 mg). The mixture was stirred at 110° C. for 4 h, cooled to rt, filtered, concentrated and purified by preparative HPLC to afford compound 14a as a white solid. LCMS (ESI): m/z 311.1 (M+H)⁺.

### Step 2: 2-((3,5-Difluoro-3′-(methoxy-d3)-[1,1′-biphenyl]-4-yl)(2-methoxy-2-oxoethyl)carbamoyl)cyclopent-1-ene-1-carboxylic acid (14b)

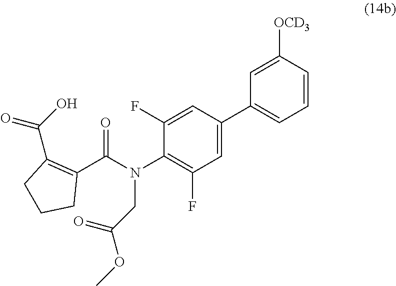

By coupling compound 14a with 1-cyclopentene-1,2-dicarboxylic anhydride similar as described above, the target compound 14b was prepared. LCMS (ESI): m/z 449.1 (M+H)⁺.

### Step 3: Methyl N-(3,5-difluoro-3′-(methoxy-d3)-[1,1′-biphenyl]-4-yl)-N-(2-(methoxy-carbamoyl)cyclopent-1-ene-1-carbonyl)glycinate (14)

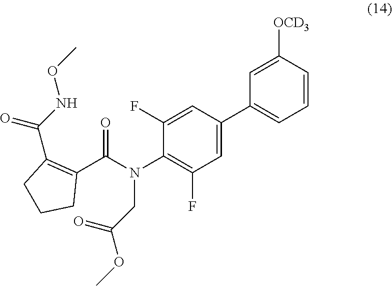

By coupling compound 14b with O-methylhydroxylamine hydrochloride similar as described above in Example 9, step 4, the target compound 14 was obtained as a white solid after purification via preparative HPLC. LCMS (ESI): m/z 478.2 (M+H)⁺.

### Example 15

### Step 1: N1-(3,5-Difluoro-3′-(methoxy-d3)-[1,1′-biphenyl]-4-yl)-N1-(2-hydroxyethyl)-N2-methoxy-cyclopent-1-ene-1,2-dicarboxamide (15)

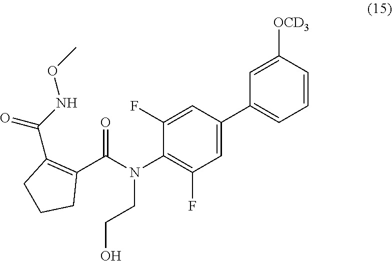

To a solution of compound 14 (150 mg) in dry THF (3 mL) was added LiAlH₄ (25 mg) at 0° C. The mixture was stirred for 2 h at this temperature, quenched by water (0.25 mL) and then diluted with 10% NaOH (0.5 mL) and water (0.75 mL). The organic layer of the reaction mixture was concentrated and purified by preparative HPLC to afford target compound 15 as a white solid. ¹H-NMR (400 MHz, MeOD-d4, mixture of E/Z-isomer) δ 7.40-7.36 (m, 3H), 7.23-7.17 (m, 2H), 6.98 (dd, J=1.8, 8.2 Hz, 1H), 3.92-3.58 (m, 7H), 2.88-2.37 (m, 4H), 2.15-1.75 (m, 2H). LCMS (ESI): m/z 450.1 (M+H)⁺.

### Example 16-1 and Example 16-2

### Step 1: Thiazole-4,5-dicarbonyl dichloride (16a)

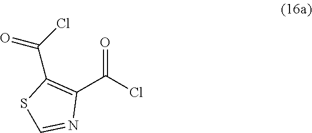 [SMILES: O=C(Cl)c1ncsc1C(=O)Cl]

To a solution of thiazole-4,5-dicarboxylic acid (200 mg) in dry DCM (2 mL) was added SOCl₂ (275 mg) at 0° C. and then the mixture was stirred at 0° C. for 2 h and concentrated under vacuum to afford the crude acid chloride intermediate 16a.

### Step 2: 4-((2,3,5,6-Tetrafluoro-3′-(methoxy-d3)-[1,1′-biphenyl]-4-yl)carbamoyl)thiazole-5-carboxylic acid (16b-1) and 5-((2,3,5,6-tetrafluoro-3′-(methoxy-d3)-[1,1′-biphenyl]-4-yl)carbamoyl)thiazole-4-carboxylic acid (16b-2)

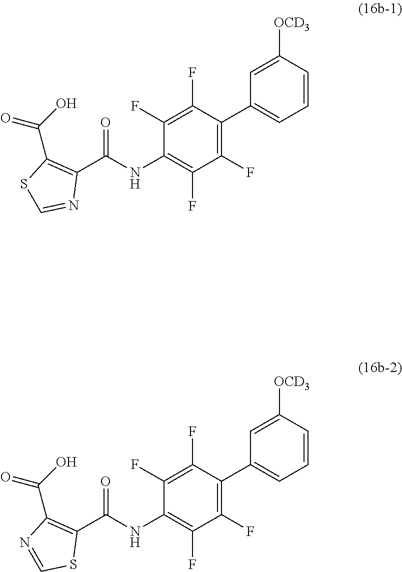

To a solution of 2,3,5,6-tetrafluoro-3′-(methoxy-d3)-[1,1′-biphenyl]-4-amine (250 mg) in dry THE (2 mL) was added NaH (219 mg, 60% wt) at 0° C. and the mixture was stirred at 0° C. for 30 min. Then intermediate 16a was added at 0° C. and the mixture was stirred at rt for 16 h, quenched with saturated aq. NH₄Cl and extracted with EA (3×5 mL). The combined organic layer was dried over Na₂SO₄, filtered and concentrated to give a mixture of compound 16b-1 and 16b-2 as a yellow solids. LCMS (ESI): m/z 483.3 (M+H)⁺.

### Step 3: N5-methoxy-N4-(2,3,5,6-tetrafluoro-3′-(methoxy-d3)-[1,1′-biphenyl]-4-yl)thiazole-4,5-dicarboxamide (16-1) and N4-methoxy-N5-(2,3,5,6-tetrafluoro-3′-(methoxy-d3)-[1,1′-biphenyl]-4-yl)thiazole-4,5-dicarboxamide (16-2)

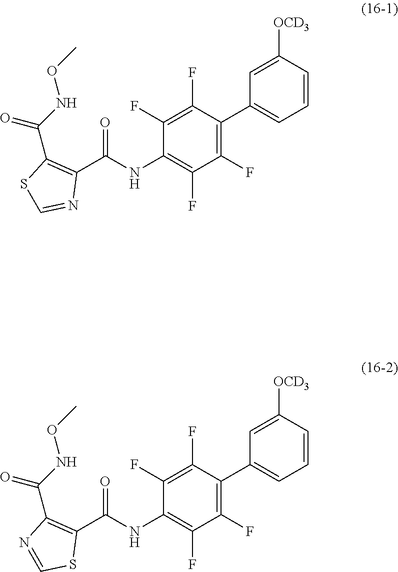

To a solution of the mixture of compound 16b-1 and 16b-2 (100 mg) in THF (1 mL) and N-methyl-2-pyrrolidone (1 mL) was added O-methylhydroxylamine (60 mg) and EDCI (92 mg). The mixture was stirred at rt for 4 h, concentrated and purified by preparative HPLC to afford separated compound 16-1 and 16-2 as white solids. 16-1: ¹H-NMR (400 MHz, DMSO-d6) δ 12.42 (s, 1H), 11.04 (s, 1H), 9.40 (s, 1H), 7.49 (t, J=8.0 Hz, 1H), 7.16-7.09 (m, 3H), 3.73 (s, 3H). LCMS (ESI): m/z 549.1 (M+H)⁺; 16-2: ¹H-NMR (400 MHz, DMSO-d6) δ 13.38 (br s, 1H), 12.72 (br s, 1H), 9.39 (s, 1H), 7.48 (t, J=8.0 Hz, 1H), 7.14-7.09 (m, 3H), 3.78 (s, 3H). LCMS (ESI): m/z 549.0 (M+H)⁺.

### Example 16/1 to 16/3

The following Examples were prepared similar as described for Example 16-1/16-2 above using the appropriate building block as shown below.

| # | building block | structure | analytical data |
|---|---|---|---|
| 16/1 | 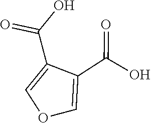 [SMILES: O=C(O)c1cocc1C(=O)O] | 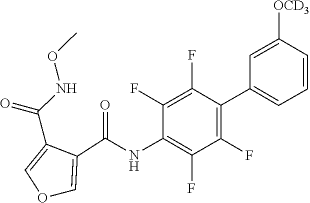 | ¹H-NMR (400 MHz, MeOD-d4) δ 8.34 (d, J = 2.0 Hz, 1H), 8.13 (s, 1H), 7.45-7.40 (m, 1H), 7.07-7.04 (m, 3H), 3.79 (s, 3H). LCMS (ESI): m/z 442.1 (M + H)⁺ |
| 16/2 | 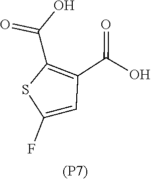 | 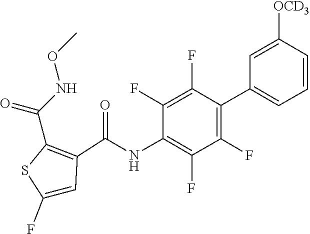 | ¹H-NMR (400 MHz, MeOD-d4) δ 7.43 (dd, J = 8.4, 7.2 Hz, 1H), 7.06-7.01 (m, 4H), 3.71 (s, 3H). LCMS (ESI): m/z 476.1 (M + H)⁺ |
| 16/3 | | 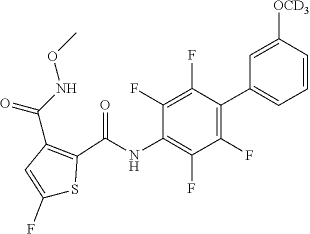 | ¹H-NMR (400 MHz, MeOD-d4) δ 7.42 (dd, J = 8.4, 7.6 Hz, 1H), 7.05-6.95 (m, 4H), 3.74 (s, 3H). LCMS (ESI): m/z 476.1 (M + H)⁺ |

### Example 17-1 and Example 17-2

### Step 1: 2-(Methoxycarbamoyl)-5-methylthiophene-3-carboxylic acid (17a-1) and 3-(methoxy-carbamoyl)-5-methylthiophene-2-carboxylic acid (17a-2)

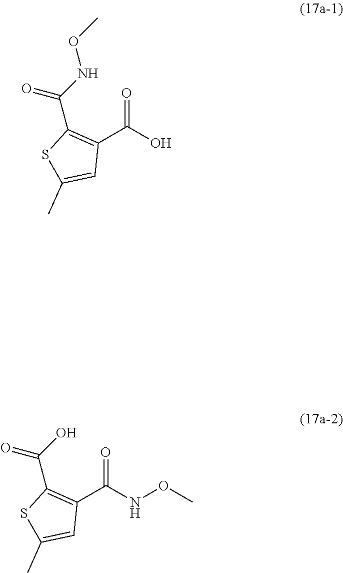

A solution of 5-methylthiophene-2,3-dicarboxylic acid (200 mg) and O-methylhydroxylamine hydrochloride (268 mg) in THF (1 mL) and N-methyl-2-pyrrolidone (1 mL) was added EDCI (411 mg) in three portions. The mixture was stirred at rt for 4 h. diluted with water and extracted with EA (3 x). The combined organic layer was dried over Na₂SO₄, filtered, concentrated and purified by reversed-phase flash chromatography (C18) (0.1% TFA in water, 10 to 100% MeCN) to give a mixture of compound 17a-1 and 17a-2 as a white solid. LCMS (ESI): m/z 215.9 (M+H)⁺.

### Step 2: N2-Methoxy-5-methyl-N3-(2,3,5,6-tetrafluoro-3′-(methoxy-d3)-[1,1′-biphenyl]-4-yl)thiophene-2,3-dicarboxamide (17-1) and N3-methoxy-5-methyl-N2-(2,3,5,6-tetrafluoro-3′-(methoxy-d3)-[1,1′-biphenyl]-4-yl)thiophene-2,3-dicarboxamide (17-2)

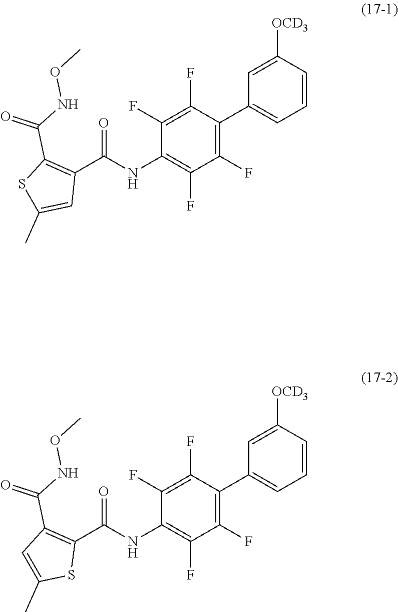

A solution of mixture 17a-1 and 17a-2 (100 mg) in dry DCM (2 mL) was added SOCl₂ (111 mg) at 0° C. Then the mixture was stirred at 0° C. for 2 h and concentrated under vacuum to afford the crude acid chloride intermediate. To a solution of 2,3,5,6-tetrafluoro-3′-(methoxy-d3)-[1,1′-biphenyl]-4-amine (127 mg) in dry THF (2 mL) was added NaH (93 mg, 60% wt) at 0° C. and the mixture was stirred at 0° C. for 10 minutes. Then the acid chloride intermediate was added at 0° C. and the mixture was stirred at rt for 16 h, quenched with saturated aq. NH₄Cl and extracted with EA (3×30 mL). The combined organic layer was dried over Na₂SO₄, concentrated and purified by reversed-phase flash chromatography (C18) (0.1% TFA in water, 10 to 100% MeCN) to give separated 17-1 and 17-2 as white solids, respectively. 17-1: ¹H-NMR (400 MHz, DMSO-d6) δ 11.99 (br s, 1H), 11.03 (br s, 1H), 7.48 (t, J=7.8 Hz, 1H), 7.36 (s, 1H), 7.15-7.08 (m, 3H), 3.69 (s, 3H), 2.53 (s, 3H). Isomer confirmed by NOESY. LCMS (ESI): m/z 472.0 (M+H)⁺; 17-2: ¹H-NMR (400 MHz, DMSO-d6) δ 12.34 (s, 1H), 12.28 (s, 1H), 7.48 (t, J=8.0 Hz, 1H), 7.24 (d, J=0.4 Hz, 1H), 7.13-7.08 (m, 3H), 3.76 (s, 3H), 2.52 (s, 3H). Isomer confirmed by NOESY. LCMS (ESI): m/z 472.0 (M+H)⁺.

### Additional Examples

The following Examples can be prepared similar as described above using the appropriate building blocks.

| building block | structure |
|---|---|
| 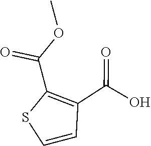 | 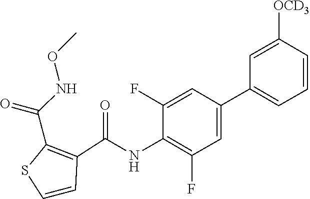 |
| 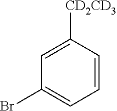 | 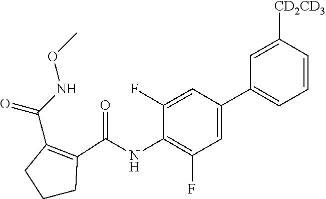 |
| 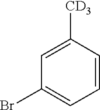 [SMILES: [2H]C([2H])([2H])c1cccc(Br)c1] | 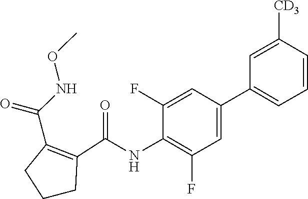 [SMILES: [2H]C([2H])([2H])c1cccc(-c2cc(F)c(NC(=O)C3=C(C(=O)NOC)CCC3)c(F)c2)c1] |
| 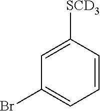 | 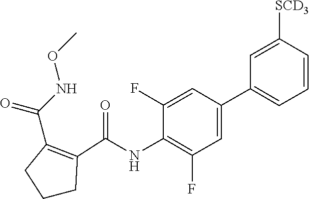 |
| 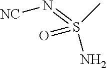 | 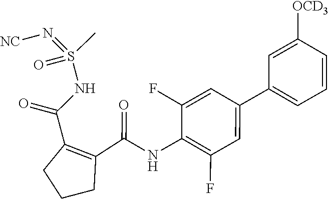 |
| via oxidation of thioether | 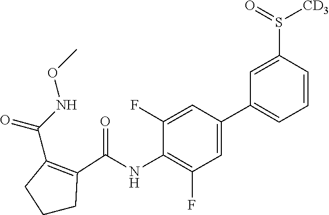 [SMILES: [2H]C([2H])([2H])S(=O)c1cccc(-c2cc(F)c(NC(=O)C3=C(C(=O)NOC)CCC3)c(F)c2)c1] |
| 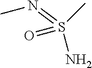 | 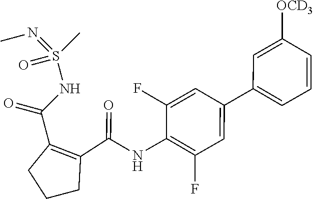 |
| 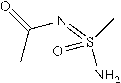 | 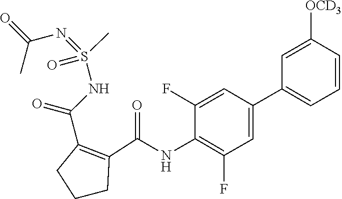 |
| | 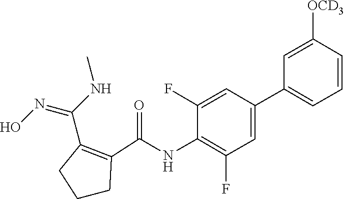 |
| | 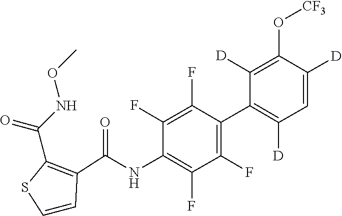 [SMILES: [2H]c1cc([2H])c(-c2c(F)c(F)c(NC(=O)c3ccsc3C(=O)NOC)c(F)c2F)c([2H])c1OC(F)(F)F] |
| | 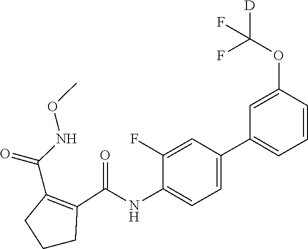 [SMILES: [2H]C(F)(F)Oc1cccc(-c2ccc(NC(=O)C3=C(C(=O)NOC)CCC3)c(F)c2)c1] |
| | 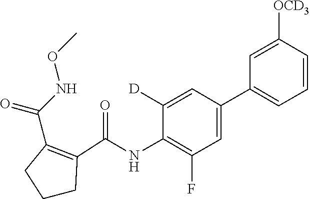 |
| | 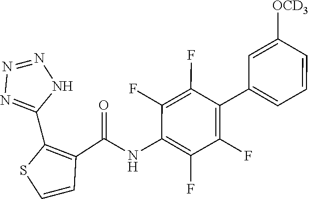 |
| | 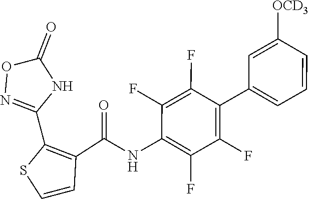 |
| | 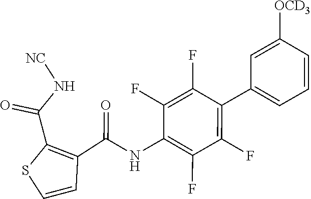 |
| | 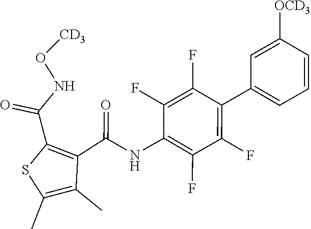 |
| | 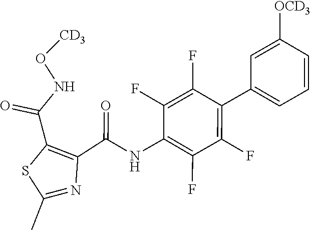 |
| | 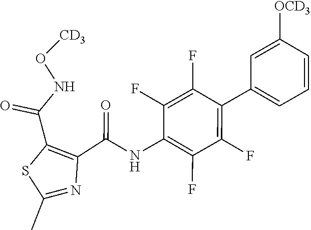 |

### Example 200: Human DHODH Inhibition Assay

The in vitro inhibition of hDHODH was measured using an N-terminally truncated recombinant hDHODH enzyme as described in J. Med. Chem. 2006; 49:1239. Briefly, the hDHODH concentration was adjusted in a way that an average slope of approximately 0.2 AU/min served as the positive control (e.g. without inhibitor). The standard assay mixture contained 60 μM 2,6-dichloroindophenol, 50 μM decylubiquinone and 100 μM dihydroorotate. The hDHODH enzyme with or without at least six different concentrations of the compounds was added and measurements were performed in 50 mM TrisHCl, 150 mM KCl and 0.1% Triton X-100 at pH 8.0 and at 30° C. The reaction was started by adding dihydroorotate and measuring the absorption at 600 nm for 2 min. For the determination of the IC₅₀ values, each data point was recorded in triplicate. Each data point was recorded in duplicate. The following data was obtained:

| Example # | IC₅₀ range |
|---|---|
| 1 | +++ |
| 1/1 | +++ |
| 1/2 | 0 |
| 1/3 | + |
| 1/4 | +++ |
| 1/5 | +++ |
| 1/6 | +++ |
| 1/7 | +++ |
| 1/8 | + |
| 1/9 | +++ |
| 1/10 | ++ |
| 1/11 | ++ |
| 1/12 | +++ |
| 1/13 | +++ |
| 1/13 | +++ |
| 2 | +++ |
| 2/1 | +++ |
| 2/2 | 0 |
| 2/3 | ++ |
| 2/4 | +++ |
| 2/5 | +++ |
| 2/6 | +++ |
| 2-1 | ++ |
| 2-1/1 | +++ |
| 3 | ++ |
| 3/1 | + |
| 3-1 | 0 |
| 3-2 | 0 |
| 4 | ++ |
| 4/1 | + |
| 4/2 | ++ |
| 4/3 | + |
| 4/4 | ++ |
| 4/5 | 0 |
| 4/8 | 0 |
| 4/9 | + |
| 4/10 | 0 |
| 4/11 | 0 |
| 4/12 | 0 |
| 4/13 | + |
| 4/14 | + |
| 4/15 | ++ |
| 4/22 | +++ |
| 4/23 | +++ |
| 4/24 | 0 |
| 4/25 | +++ |
| 4/26 | +++ |
| 4/27 | ++ |
| 4/28 | +++ |
| 4/29 | +++ |
| 4/30 | ++ |
| 4/31 | +++ |
| 4/32 | +++ |
| 4/33 | + |
| 5 | ++ |
| 5/1 | +++ |
| 5/2 | ++ |
| 5/3 | ++ |
| 5/4 | +++ |
| 5/5 | +++ |
| 5/6 | ++ |
| 5/7 | 0 |
| 5/8 | + |
| 5/9 | +++ |
| 5/10 | +++ |
| 5/11 | +++ |
| 5/12 | +++ |
| 5/13 | +++ |
| 5/14 | +++ |
| 5/15 | +++ |
| 5/16 | +++ |
| 6 | +++ |
| 6/1 | +++ |
| 6/2 | +++ |
| 6/3 | +++ |
| 8 | + |
| 8/1 | 0 |
| 8/2 | + |
| 8/3 | + |
| 9 | +++ |
| 9/1 | +++ |
| 9/2 | +++ |
| 9/3 | +++ |
| 9/4 | +++ |
| 9/5 | +++ |
| 9/6 | +++ |
| 9/7 | +++ |
| 9/8 | +++ |
| 9/9 | +++ |
| 10 | +++ |
| 10/1 | +++ |
| 13 | 0 |
| 14 | + |
| 15 | 0 |
| 16-1 | +++ |
| 16-2 | ++ |
| 16/1 | +++ |
| 16/2 | +++ |
| 16/3 | +++ |
| 17-1 | +++ |
| 17-2 | +++ |

IC₅₀ ranges for the human DHODH assay as described herein: +++: <100 nM; ++: 100 nM to <1 μM; +: 1 μM to <10 μM; 0: ≥10 μM.

### Matched Pair Comparison:

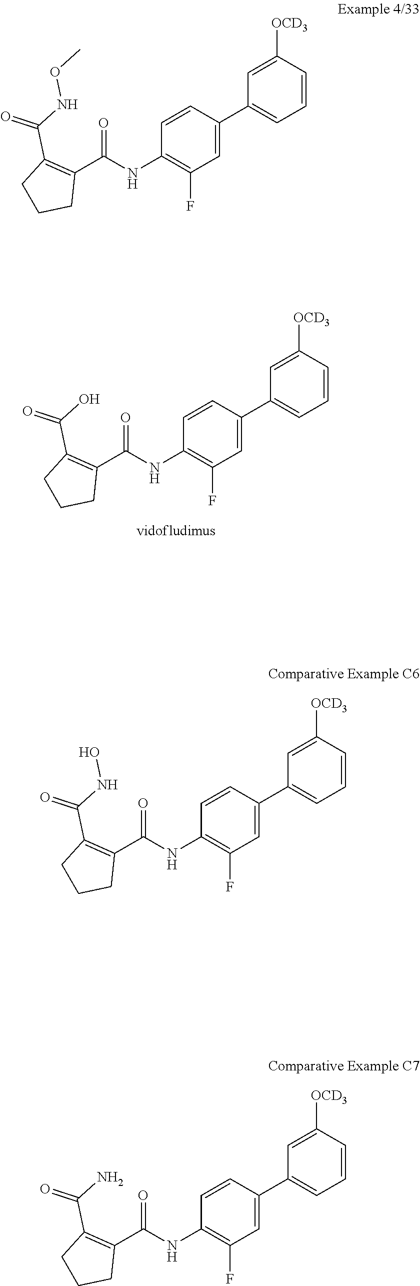

| Example # | DHODH IC₅₀ | |
|---|---|---|
| 4/33 | 2.02 | μM |
| vidofludimus (Comparative | 0.554 | μM |
| Example) | | |
| Comparative Example C6 | >50 | μM |
| Comparative Example C7 | ca. 10 | μM |

Conclusion: Example 4/33 has a similar DHODH inhibition as the matched pair carboxylic acid (vidofludimus) while the matched pair hydroxamate (Comparative Example C6, equals Example 4 in WO2004/056746) is much less potent, similar as mentioned before. Also the matched pair carboxamide (Comparative Example C7) is only a weakly DHODH inhibitor. FIG. 2 shows representative human DHODH inhibition curves for this experiment.

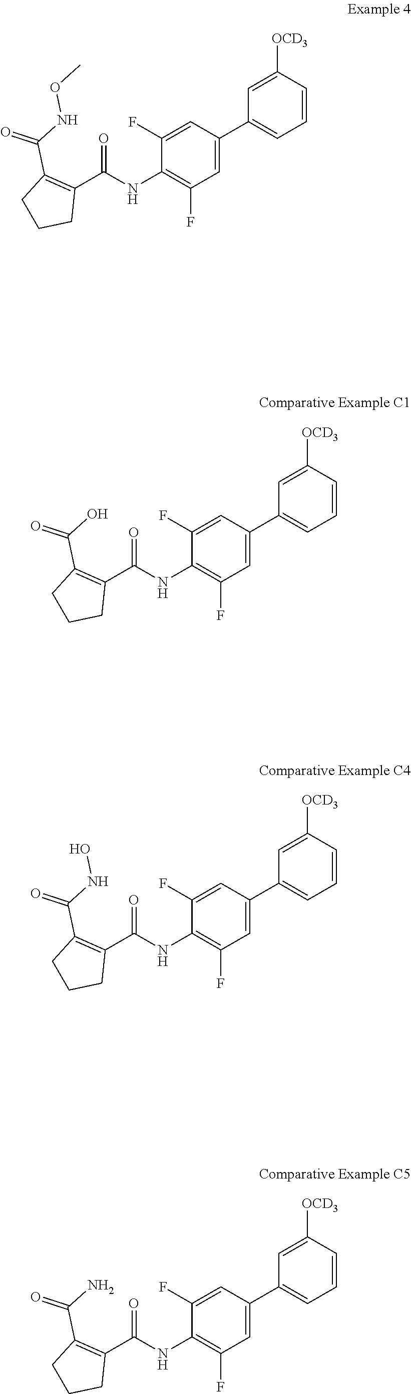

| Example # | DHODH IC₅₀ | |
|---|---|---|
| 4 | 0.174 | μM |
| Comparative Example C1 | 0.061 | μM |
| Comparative Example C4 | 14.0 | μM |
| Comparative Example C5 | 16.1 | μM |

Conclusion: A similar trend can be observed for Example 4, which has a similar DHODH inhibition as the matched pair carboxylic acid (Comparative Example C1) while the matched pair hydroxamate (Comparative Example C4) is much less potent. Similar applies to the matched pair carboxamide (Comparative Example C5), which shows also only a weak DHODH inhibition.

### Example 201: In Vitro Interaction Studies of DHODH Inhibitors with the Human URAT1 Uptake Transporters

In the human URAT1 uptake transporter assay (host cell line: MDCKII), the accumulation of the probe substrate in the presence of a DHODH inhibitor (at a concentration of 10 μM) into cells was measured. The assay was executed at SOLVO with catalogue number MDCKII-URAT1-LV. 20 μM uric acid served as probe substrate. Reference inhibitor was benzbromarone at a concentration of 300 μM, which served as internal control. The following data was obtained:

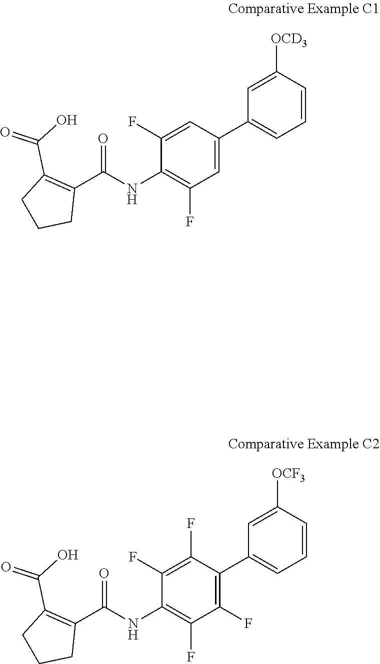

| | human URAT1 uptake transporter |
|---|---|
| Example # | stimulation @ 10 μM |
| Comparative Example C1 | 173% |
| 1/1 | <20% |
| 2/1 | 22% |
| 4 | 26% |
| Comparative Example C2 | 64% |
| 1 | 28% |
| 2 | 72% |
| 3 | <20% |

Conclusion: Comparative Example C1 (containing a carboxylic acid moiety) stimulated the URAT1-mediated uric acid accumulation up to unfavourable 173% at the investigated conditions while the matched pairs with a carboxylic acid bioisosteric moiety stimulated the URAT1-mediated uric acid accumulation to a minor extent, i.e. in a range from <20 to 26%. A similar trend could be observed for Comparative Example C2, were at least the matched pair with a N-(methylsulfonyl)carboxamide (Example 1) or with a tetrazole moiety (Example 3) instead of a carboxylic acid stimulated the URAT1-mediated uric acid accumulation to a minor extent, i.e. 28% and <20%, respectively. In summary, the examples from the present invention show less interaction with the URAT1 transporter compared to the carboxylic acid matched pairs and thus less disturbs the uric acid homeostasis, reducing the risk of occurrence of hematuria.

### Example 202a: A-B and B-A Permeability (Caco-2, pH 7.4/7.4)

The Caco-2 cell line is a human colon adeno-carcinoma cell line that differentiates in culture and resembles the epithelial lining of the human small intestine. The apparent permeability (Pₐₚₚ) of the test compound at 10 μM across the Caco-2 monolayer in both direction was measured using the standard protocol from Eurofins Discovery Services (Item #3319 and 3321). The following data was obtained:

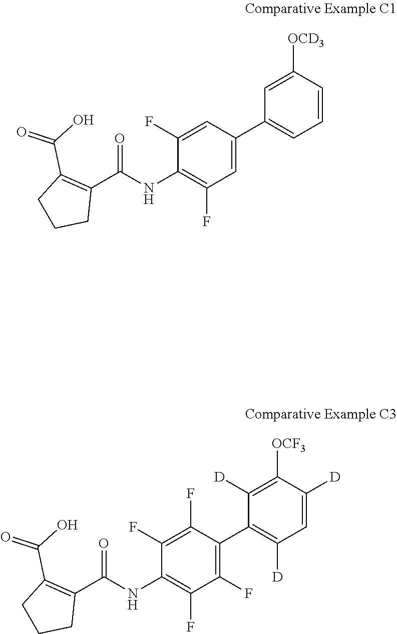

| Example # | A-B permeability | recovery | B-A permeability | recovery |
|---|---|---|---|---|
| C1 | 0.68 nm/s | 72% | 8.8 nm/s | 69% |
| 4 | 16 nm/s | 60% | 2.6 nm/s | 52% |

The absorption of orally administered drugs requires the movement of the drug across the intestinal epithelial barrier. Intestinal permeability is a critical characteristic that determines the rate and extent of in vivo absorption and is correlated with the bioavailability of a drug candidate. While the Comparative Example C1 (containing a carboxylic acid moiety) has a low permeability, the matched pair with a carboxylic acid bioisosteric moiety (Example 4) has a much higher permeability from the apical (A) to basal (B) compartment.

### Example 202b: Kinetic Aqueous Solubility and Log D

The kinetic aqueous solubility in PBS at pH 7.4 was determined by comparing the peak area of the principal peak in a calibration standard (200 μM) containing organic solvent (MeOH/water, 60/40 v/v) with the peak area of the corresponding peak in the PBS buffer sample. In addition, chromatographic purity (%) was defined as the peak area of the principal peak relative to the total integrated peak area in the HPLC chromatogram of the calibration standard. A chromatogram of the calibration standard of each test compound, along with a UV/VIS spectrum with labeled absorbance maxima, was generated. Kinetic aqueous solubility was measured at a wavelength of 230 nm using the standard protocol from Eurofins Discovery Services (Item #435).

The total amount of compound was determined as the peak area of the principal peak in a calibration standard (100 μM) containing organic solvent (MeOH/water, 60/40 v/v). The amount of compound in buffer was determined as the combined, volume-corrected and weighted areas of the corresponding peaks in the aqueous phases of three organic-aqueous samples of different composition. An automated weighting system was used to ensure the preferred use of raw data from those samples with well quantifiable peak signals. The amount of compound in organic was calculated by subtraction.

Subsequently, the partition coefficient (log D, n-octanol/PBS at pH 7.4) was calculated as the Log₁₀ of the amount of compound in the organic phase divided by the amount of compound in the aqueous phase (Eurofins Discovery Services Item #417). The following data was obtained (n.t.=not tested):

| Example # | solubility | logD |
|---|---|---|
| Comparative Example C1 | 220 μM | 1.0 |
| 1/1 | 227 μM | 0.9 |
| 2/1 | 235 μM | 0.5 |
| Comparative Example C3 | 187 μM | 2.6 |
| 1 | 237 μM | n.t. |
| 3 | 207 μM | n.t. |

The lower values for the distribution coefficient log D for the examples from the present invention compared to the carboxylic acid matched pairs indicate, that the compound is to a higher extent in an aqueous environment (such as blood serum) compared to a lipophilic environment (such as lipid bilayer), which is beneficial for its druglikeness and pharmacokinetics. The examples from the present invention have also a higher aqueous solubility compared to the carboxylic acid matched pairs.

### Example 203: Antiviral Activity on SARS-CoV-2

The assay for viral replication (YFP) and the cell viability assay has been described in general in Pathogens 2021; 10:1076 and applied to compounds of the present invention furnished the following results: EC₅₀ ranges for the SARS-CoV-2 assay as described herein:

| Example # | EC₅₀ range | CC₅₀ range |
|---|---|---|
| 1 | ++ | >100 |
| 1/1 | + | >100 |
| 1/2 | ++ | >100 |
| 1/3 | + | 72 |
| 1/4 | 0 | >100 |
| 1/5 | +++ | >100 |
| 1/6 | + | >100 |
| 1/7 | +++ | >100 |
| 1/12 | ++++ | >100 |
| 1/14 | 0 | >100 |
| 2 | ++ | >100 |
| 2/1 | 0 | >100 |
| 2/2 | ++ | >100 |
| 2/3 | ++ | >100 |
| 2/4 | +++ | >100 |
| 2/5 | ++++ | >100 |
| 2/6 | +++ | >100 |
| 2-1 | ++ | >100 |
| 2-1/1 | ++ | 55 |
| 3 | +++ | >100 |
| 3/1 | + | 48 |
| 3-1 | +++ | >100 |
| 3-2 | +++ | >100 |
| 4 | +++ | >100 |
| 4/1 | +++ | >100 |
| 4/2 | +++ | >100 |
| 4/3 | ++ | >100 |
| 4/4 | +++ | >100 |
| 4/9 | +++ | >100 |
| 4/11 | + | >100 |
| 4/12 | +++ | >100 |
| 4/13 | +++ | >100 |
| 4/14 | +++ | >100 |
| 4/15 | ++ | >100 |
| 4/22 | ++++ | >100 |
| 4/23 | ++++ | >100 |
| 4/25 | ++++ | >100 |
| 4/26 | ++++ | >100 |
| 4/27 | ++++ | >100 |
| 4/28 | ++++ | >100 |
| 4/29 | ++++ | >100 |
| 4/30 | +++ | >100 |
| 4/31 | ++++ | >100 |
| 4/32 | ++++ | >100 |
| 5 | +++ | >100 |
| 5/1 | ++++ | 59 |
| 5/2 | ++++ | >100 |
| 5/3 | +++ | >100 |
| 5/4 | ++++ | >100 |
| 5/5 | ++++ | >100 |
| 5/6 | +++ | >100 |
| 6 | ++++ | >100 |
| 6/1 | ++++ | >100 |
| 6/2 | +++ | >100 |
| 6/3 | ++++ | >100 |
| 8 | 0 | >100 |
| 8/1 | +++ | 100 |
| 8/2 | +++ | >100 |
| 8/3 | +++ | >100 |
| 9 | ++++ | >100 |
| 9/1 | ++++ | >100 |
| 9/2 | ++++ | >100 |
| 9/3 | ++++ | 48 |
| 9/4 | ++++ | >100 |
| 9/5 | ++++ | 81 |
| 9/6 | ++++ | 70 |
| 9/7 | ++++ | 94 |
| 9/8 | ++++ | 68 |
| 10 | ++++ | >100 |
| 10/1 | ++++ | >100 |
| 13 | ++ | >100 |
| 16/1 | ++++ | >100 |
| 17-1 | ++++ | 60 |
| 17-2 | ++++ | 72 |

EC₅₀ ranges for the SARS-CoV-2 assay as described herein:
++++: <1 μM;
+++: <10 μM;
++: 10 μM to <25 μM;
+: 25 μM to <50 μM;
0: ≥50 μM.

### Example 204: Synergistic Antiviral Activity on SARS-CoV-2 with a Nucleoside Analogue

The synergistic potential of Example 1 together with the nucleoside analogue EIDD-1931 (CAS: 3258-02-4) was assessed.

The method of combinatorial drug assessment by a viral replication inhibition assay has been published in Pathogens 2021; 10:1076. Caco-2 cells were cultivated in 96-well plates at 25000 cells/well, infected with SARS-CoV-2 d6-YFP at an MOI of 0.003 and treated with Example 1, EIDD-1931 or a combination of the drugs, starting at the respective 4×EC₅₀ concentrations of the single compounds.

Viral replication was determined as 30 h post infection (p.i.) by quantitative fluorescence detection of virus-driven YFP expression in the fixed cells. Inhibitory profiles of viral replication measured through virus-encoded YFP reporter expression are presented in a bar chart of quadruplicate determinations (mean±SD). The combinatorial drug assessment was calculated by using the CompuSyn algorithm as described in Int. J. Mol. Sci. 2021; 22:575.

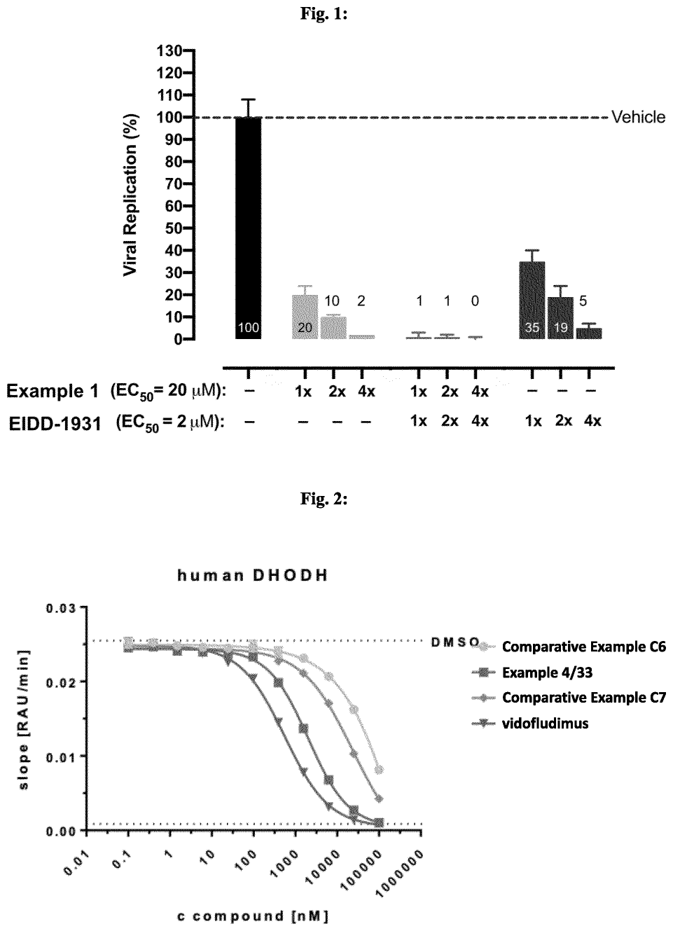

A representative experiment is shown in FIG. 1. Compound 1 shows synergistic antiviral effects on SARS-CoV-2 when combined with nucleoside analogue EIDD-1931 (CAS: 3258-02-4).

### Example 205: Mouse Pharmacokinetics

The pharmacokinetics of the compounds of the present invention was evaluated in 3 male and 3 female mice (C57BL/6J, 8 week old) after oral or intravenous cassette dosing to assess the oral bioavailability. Dose was 5 mg/kg (oral) and 1 mg/kg (intravenous), application volume was 5 mL/kg (oral) and 0.5 mL/kg (intravenous), vehicle was 5% solutol, 95% NaCl solution (at 0.9% saline concentration) for oral and 5% solutol, 5% ethanol, 90% NaCl solution (at 0.9% saline concentration) for intravenous. At each designated time point (0.5, 1, 2, 4, 8 and 24 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

| Example # | Cₘₐₓ | t_{1/2} | AUC | F |
|---|---|---|---|---|
| 1 | ♂ 754 ng/mL | ♂ 2.9 h | ♂ 3200 ng*h/mL | ♂ 49% |
| | ♀ 1320 ng/mL | ♀ 2.8 h | ♀ 7350 ng*h/mL | ♀ 217% |
| 2/1 | ♂ 1500 ng/mL | ♂ 3.1 h | ♂ 3070 ng*h/mL | ♂ 60% |
| | ♀ 2280 ng/mL | ♀ 2.4 h | ♀ 8610 ng*h/mL | ♀ 144% |
| 4 | ♂ 1110 ng/mL | ♂ 1.1 h | ♂ 1780 ng*h/mL | ♂ 63% |
| | ♀ 690 ng/mL | ♀ 1.3 h | ♀ 1210 ng*h/mL | ♀ 87% |

Abbreviations:
♂ = male,
♀ = female,
Cₘₐₓ = peak plasma concentration,
t_{1/2} = elimination half-life,
AUC = area under the curve (integral of the concentration-time curve from 0 to 24 h),
F = bioavailability (systemically available fraction)

Conclusion: The experiment shows that good PK properties can be obtained with bioisosters of carboxylic acids.

In another matched pair analysis the benefit of deuteration of the N-methoxyacetamide moiety was tested: the pharmacokinetic properties of the compounds were evaluated in 3 female mice (C57BL/6J, 8 week old) after oral or intravenous cassette dosing to assess the oral bioavailability. Dose was 5 mg/kg (oral) and 1 mg/kg (intravenous), application volume was 5 mL/kg (oral) and 2 mL/kg (intravenous), vehicle was 5% solutol, 95% NaCl solution (at 0.9% saline concentration) for oral and 5% solutol, 5% ethanol, 90% NaCl solution (at 0.9% saline concentration) for intravenous application. At each designated time point (0.25, 0.5, 1, 2, 4 and 8 h after dosing for oral; 0.083, 0.25, 0.5, 1, 4 and 8 h after dosing for intravenous), 20 μL whole blood were collected from the tail vein into Li-heparin tubes, frozen on dry ice within 1-2 minutes of sampling and stored at −20° C. until processed for LC-MS analysis. The obtained data is as follows:

| Example # | Cₘₐₓ | t_{1/2} | | AUC_{-8 h} | F |
|---|---|---|---|---|---|
| 9 | 3000 ng/mL | 2.6 | h | 12300 ng*h/mL | 49% |
| 9/6 | 6700 ng/mL | 2.5 | h | 25000 ng*h/mL | 76% |
| 5/4 | 18000 ng/mL | >8 | h | 106000 ng*h/mL | 39% |
| 5/5 | 70000 ng/mL | >8 | h | 165000 ng*h/mL | 56% |

Abbreviations:
Cₘₐₓ = peak blood concentration,
t_{1/2} = elimination half-life,
AUC_{-8 h} = area under the curve (integral of the concentration-time curve from 0 to 8 h),
F = bioavailability (systemically available fraction)

Conclusion: The metabolic stability can be improved by selective deuteration at a vulnerable position (i.e. the N-methoxyacetamide moiety).

### Example 206: Antiviral Activity on SARS-CoV-2 Variants of Concern

Antiviral activity of Example 9 against Delta and Omicron variants of concern was tested similar as for SARS-CoV-2 WT. Caco-2 cells were treated with serial dilutions of the indicated compound and then infected with SARS-CoV-2 reporter virus d6-YFP (WT) or clinical isolates of the Delta or Omicron variants. The number of infected cells was quantified by YFP expression for the WT or immunofluorescence staining with a dsRNA-specific antibody and a fluorophore-coupled secondary antibody and the respective EC₅₀ concentration was calculated. The following results were obtained:

| Example # | EC₅₀ WT | EC₅₀ Delta | EC₅₀ Omikron |
|---|---|---|---|
| 9 | 0.072 μM | <0.003 μM | <0.003 μM |

### Example 207: Antiviral Activity on Respiratory Syncytial Virus (RSV)

Hep-2 cells were treated with Example 9 or DMSO for two days and cell viability was quantified by measuring intracellular ATP levels using the CellTiter-Glo Lumienscent Cell Viability Assay (Promega). Mean values from triplicates relative to DMSO control f SD are determined. The 50% cytotoxic concentration (CC₅₀) was calculated via non-linear regression analysis using GraphPad Prism. Again, Hep-2 cells were treated with Example 9 or DMSO and infected with three different RSV strains. Infected cells were quantified two days after infection via internal GFP fluorescence (RSV-A2) or ICC staining with an RSV-specific antibody (RSV-Long and RSV-B). Mean values from triplicates relative to DMSO control±SD are determined. The 50% inhibitory concentration (IC₅₀) was calculated via nonlinear regression analysis using GraphPad Prism. The following results were obtained:

| Example # | RSV-A2 IC₅₀ | RSV-Long IC₅₀ | RSV-B IC₅₀ | CC₅₀ |
|---|---|---|---|---|
| 9 | <0.1 μM | <0.1 μM | <0.1 μM | 45.3 μM |

### Example 208: Antiviral Activity on Human Rhinovirus

Antiviral activity of compound of the present invention has also been tested on human rhinovirus HRV-14. The following results were obtained:

| | | |
|---|---|---|
| Example # | EC₅₀ | CC₅₀ |

| | | | | |
|---|---|---|---|---|
| 4 | 2.2 | μM | >25 | μM |
| 9 | 0.014 | μM | >25 | μM |
| pirodavir | 0.0003 | μM | 4.5 | μM |

### Example 209: Metabolic Stability in Rat and Human Microsomes

Example 9 (deuterated at one position) and Example 9/1 (deuterated at two positions) and the non-deuterated matched pair (Example 9/9) were incubated using two different batches of pooled SD-rat liver microsomes (RLM) and human liver microsomes (HLM), respectively, for a period of 60 min. A similar procedure was applied with Example 1/7 and its non-deuterated matched pair Example 1/13. The metabolism was monitored by HPLC-MS/MS. Verapamil served as positive control. The intrinsic clearance Clᵢₙₜ was calculated from the measured remaining compound values (in duplicate) at 0, 10, 30 and 60 minutes. The data points for 60 minutes is as follows:

| | rat liver microsomes batch | | human liver microsomes batch | |
|---|---|---|---|---|
| | 2110178 | 1110040 | 2110108 | 1210079 |
| Clᵢₙₜ | (Xenotech | (Xenotech | (Xenotech | (Xenotech |
| (μl/min/mg protein) | R1000, male) | R1500, female) | H1000, male) | H1500, female) |
| Example 9/9 | 192 | 69.3 | 93.9 | 66.1 |
| Example 9 | 183 | 63.8 | 76.5 | 55.5 |
| Example 9/1 | 162 | 52.8 | 67.2 | 40.4 |
| Example 1/13 | 25.5 | 35.4 | 14.4 | 10.3 |
| Example 1/7 | 20.0 | 30.5 | 1.32 | 4.01 |
| Verapamil | 79.8-85.2 | 27.8-28.7 | 45.3-61.6 | 33.2-54.4 |

Conclusion: The metabolic stability can be improved by selective deuteration at vulnerable positions (especially the anisole alkyl moiety). Further improvement in stability can be obtained by additional deuteration of the N-methoxyacetamide moiety (Example 9 to Example 9/1).

### Example 210: Rat Pharmacokinetics

The pharmacokinetics of compounds of the present invention was evaluated in 3 female Sprague Dawley rats (8 week old) after oral cassette dosing (vehicle: 5% solutol/95% NaCl solution (at 0.9% saline concentration; application volume: 5 mL/kg) to asses the exposure of the test items. At each designated time point (0.25, 0.5, 1, 2, 4 and 8 h after dosing), 20 μL blood were collected from the tail vein into Li-heparin tubes, cooled on ice and stored at −20° C. until processed for LC-MS analysis. The obtained data is as follows:

| Example # | Cₘₐₓ | t_{1/2} | AUC_{-8 h} | AUC_{-inf} |
|---|---|---|---|---|
| 5/9 | 17 μM | 6.6 h | 91 μM*h | 185 μM*h |
| 5/4 | 21 μM | 7.9 h | 125 μM*h | 271 μM*h |
| 5/5 | 19 μM | 8.4 h | 113 μM*h | 252 μM*h |

Abbreviations:
Cₘₐₓ = peak blood concentration,
t_{1/2} = elimination half-life,
AUC_{-8 h} = area under the curve (integral of the concentration-time curve from 0 to 8 h),
AUC_{-inf} = area under the curve calculated to infinity

Conclusion: The metabolic stability from microsomes (Example 209) translates well to improved bioavailability in an in-vivo PK study. Again, the deuterated derivatives Example 5/4 and Example 5/5 are more stable and have a better bioavailability compared to the non-deuterated matched pair Example 5/9.

1. A compound of Formula (I): 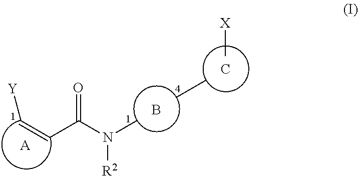
or an enantiomer, diastereomer, tautomer, solvate, or pharmaceutically acceptable salt thereof, wherein
A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, —CN, —NO₂, oxo, —OH, C₁₋₄-alkyl, —O—C₁₋₄-alkyl, fluoro-C₁₋₄-alkyl and —O-fluoro-C₁₋₄-alkyl, ring A having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²³)ₘR²¹, C₀₋₆-alkylene-NR²¹S(═O)ₓ(═NR²³)_{y}R²¹, C₀₋₆-alkylene-S(═O)ₓ(═NR²³)_{y}NR²¹R²², C₀₋₆-alkylene-NR²¹S(═O)ₓ(═NR²³)_{y}NR²¹R²², C₀₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹, C₀₋₆-alkylene-NR²¹R²²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and wherein the residue —NR² on ring B is in a 1,4-orientation with respect to ring C, B having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
C having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X having one or more hydrogen atoms optionally replaced by deuterium;
Y is selected from —CONH—CN, —CONHOH, —CONHOR¹⁰, —CONR¹⁰OH, —C(═NOH)NR¹¹R¹², —CONHS(═O)ₓ(═NR¹³)_{y}R¹⁰, —CONHS(═O)_{y}(═NR¹³)_{y}NR¹¹R¹², —SO₃H, —S(═O)ₓ(═NR¹³)_{y}NHCOR¹⁰, —S(═O)ₓ(═NR¹³)_{y}NHR¹¹, —P(═O)(OH)₂, —P(═O)(NR¹¹R¹²)OH, —P(═O)R¹¹(OH), —B(OH)₂, 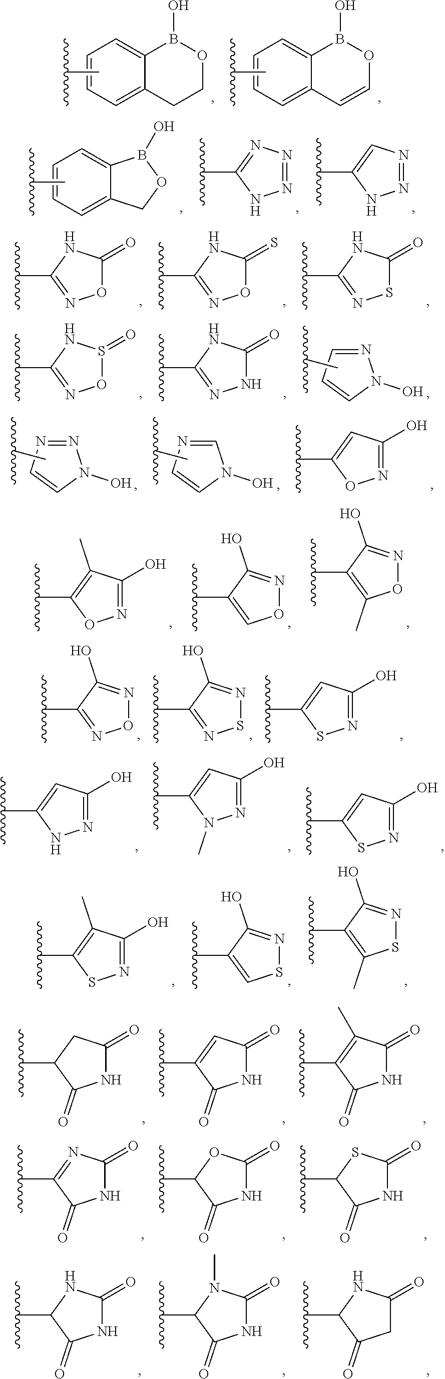 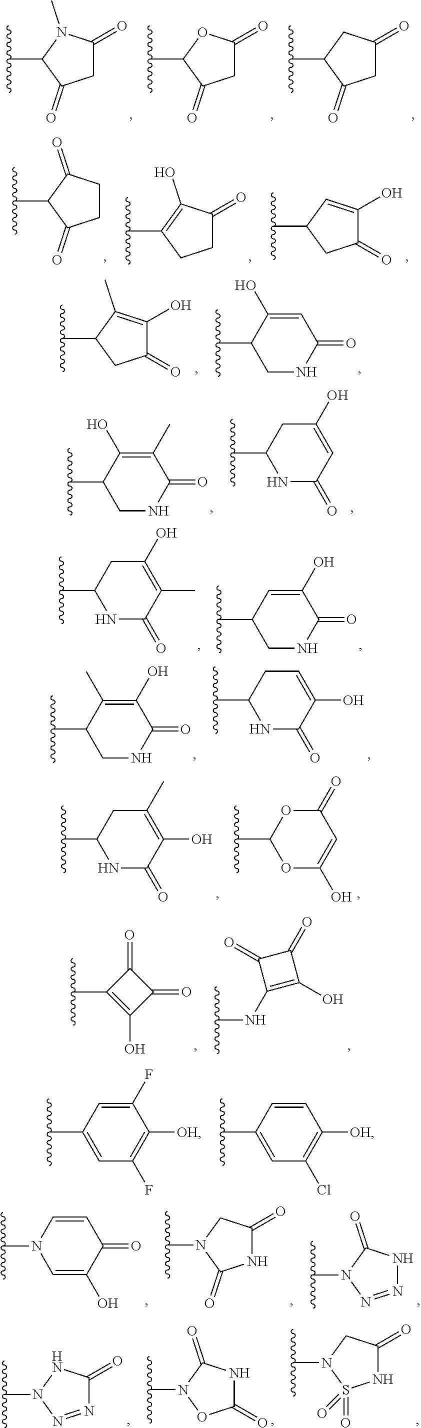 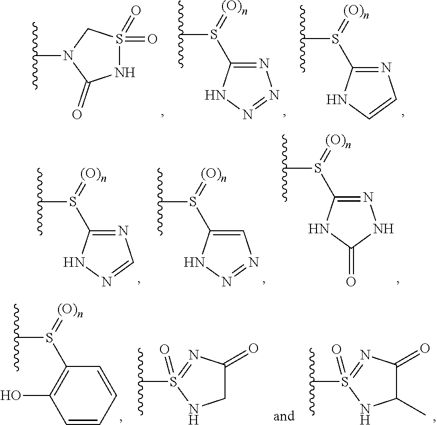
Y having one or more hydrogen atoms optionally replaced by deuterium;
R² is selected from H and C₁₋₆-alkyl,
wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² having one or more hydrogen atoms optionally replaced by deuterium;
R¹⁰ is selected from C₁₋₆-alkyl, 3- to 6-membered cycloalkyl or 3- to 6-membered heterocycloalkyl,
wherein alkyl, cycloalkyl and heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹⁰ having one or more hydrogen atoms optionally replaced by deuterium;
R¹¹, R¹², R²¹, R²², R³¹, R³², R⁴¹, R⁴² are independently selected from H, C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹¹ and/or R¹² and/or R²¹ and/or R²² and/or R³¹ and/or R³² and/or R⁴¹ and/or R⁴² having one or more hydrogen atoms optionally replaced by deuterium;
or R¹¹ and R¹², R²¹ and R²², R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R¹¹ and/or R¹² and/or R²¹ and/or R²² and/or R³¹ and/or R³² and/or R⁴¹ and/or R⁴² having one or more hydrogen atoms optionally replaced by deuterium;
R¹³, R²³, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R¹³ and/or R²³ and/or R³³ and/or R⁴³ having one or more hydrogen atoms optionally replaced by deuterium;
n, m, x, y are independently selected from 0 to 2;
with the proviso that the sum of integer m and n for the residue linked to the same sulfur atom is independently selected from 0 to 2;
with the proviso that the sum of integer x and y for the residue linked to the same sulfur atom is independently selected from 1 or 2;
and with the proviso, that the following structure is excluded:

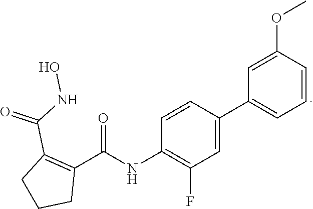 [SMILES: COc1cccc(-c2ccc(NC(=O)C3=C(C(=O)NO)CCC3)c(F)c2)c1]

2. A compound of Formula (I) according to claim 1, or a solvate or pharmaceutically acceptable salt thereof, wherein
Y is selected from —CONH—CN, —CONHOR¹⁰, —C(═NOH)NR¹¹R¹², —CONHS(═O)ₓ(═NR¹³)_{y}R¹⁰, —CONHS(═O)_{y}(═NR¹³)_{y}NR¹¹R¹², 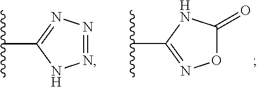 [SMILES: *c1nnn[nH]1 ; *c1noc(=O)[nH]1]
R¹⁰ is selected from C₁₋₃-alkyl, cyclopropyl or oxetan-3-yl,
wherein alkyl, cyclopropyl or oxetan-3-yl is unsubstituted or substituted with 1 to 3 substituents independently selected from F, —CN, Me, CHF₂, CF₃, —OH, oxo, —OMe, —OCHF₂ and —OCF₃, R¹⁰ having one or more hydrogen atoms optionally replaced by deuterium;
R¹¹ and R¹² are independently selected from H or C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹¹ and/or R¹² having one or more hydrogen atoms optionally replaced by deuterium;
R¹³ is selected from H, —CN and C₁₋₃-alkyl,
wherein alkyl is unsubstituted or substituted with 1 to 3 substituents independently selected from F, —CN, Me, CHF₂, CF₃, —OH, oxo, —OMe, —OCHF₂ and —OCF₃, R¹³ having one or more hydrogen atoms optionally replaced by deuterium;
x is 1 and y is 1 or x is 2 and y is 0.

3. A compound of Formula (I) according to claim 1, wherein 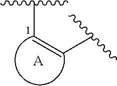
is selected from 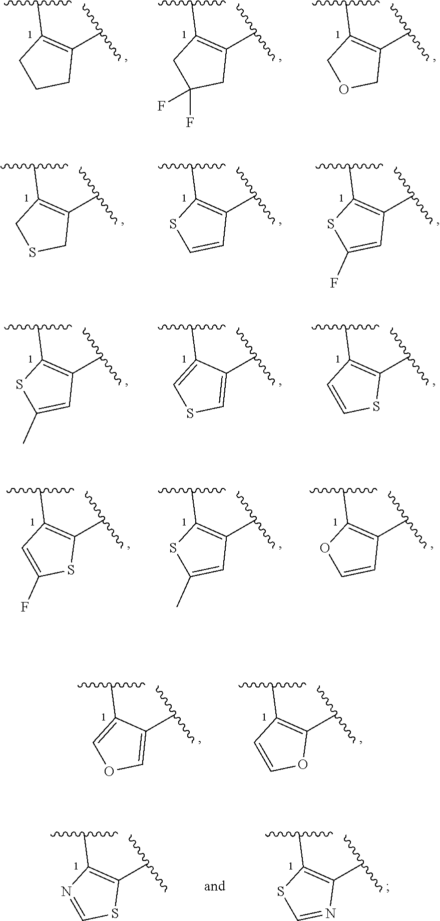
and R² is H.

4. A compound of Formula (I) according to claim 1, wherein one or more hydrogen atom(s) in any substituent is replaced by deuterium.

5. A compound of Formula (I) according to claim 1, wherein
B is phenyl,
wherein phenyl is unsubstituted or substituted with 1 to 4 substituents independently selected from the group consisting of D, F, Cl, —CN, Me, CD₃, CHF₂ and CF₃;
and wherein the residue —NR² on ring B is in a 1,4-orientation with respect to ring C.

6. A compound of Formula (I) according to claim 1, wherein
C is phenyl,
wherein phenyl is unsubstituted or substituted with 1 to 4 substituents independently selected from the group consisting of D, F, Cl, —CN, Me, CD₃, CHF₂, CF₃, —OMe, —OCD₃, —OCHF₂ and —OCF₃;
X is selected from D, F, Cl, —CN, Me, CD₃, CHF₂, CF₃, Et, CD₂CD₃, —OMe, —OCD₃, —OCHF₂, —OCF₃, —OEt and —OCD₂CD₃.

7. A compound of Formula (I) according to claim 1, wherein 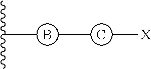
is selected from 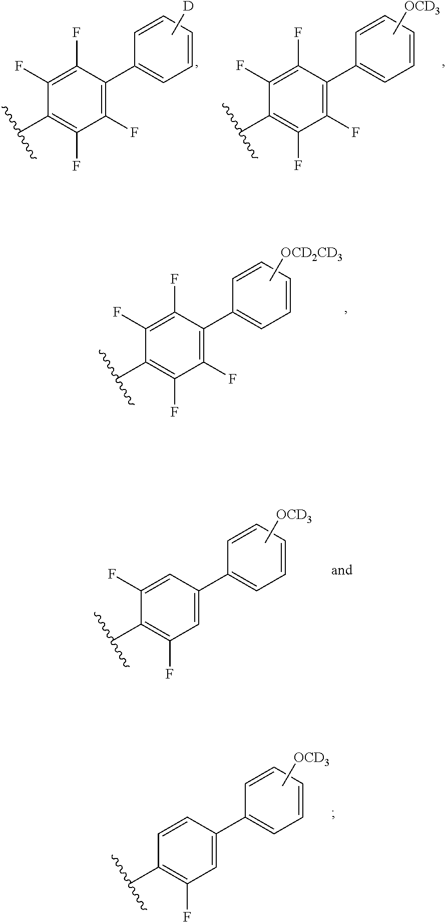
wherein ring C is optionally substituted with 1 to 4 substituents independently selected from D or F.

8. A compound of Formula (I) according to claim 1, wherein
Y is selected from 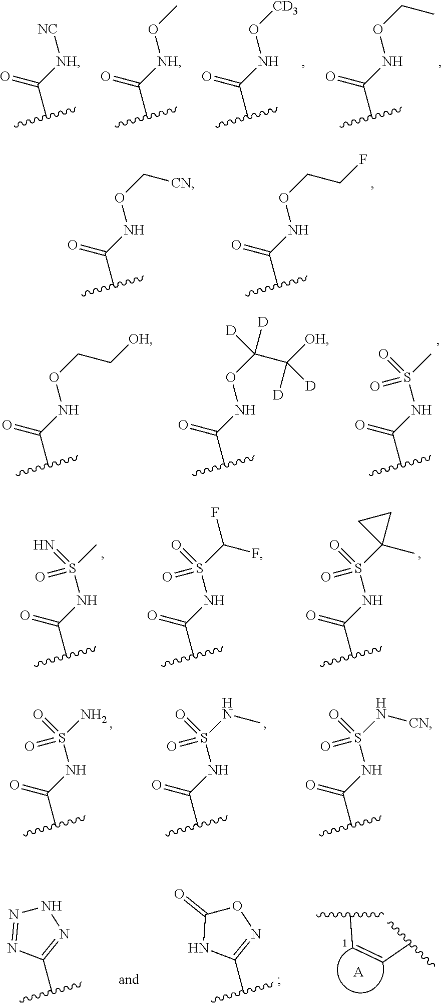
is selected from 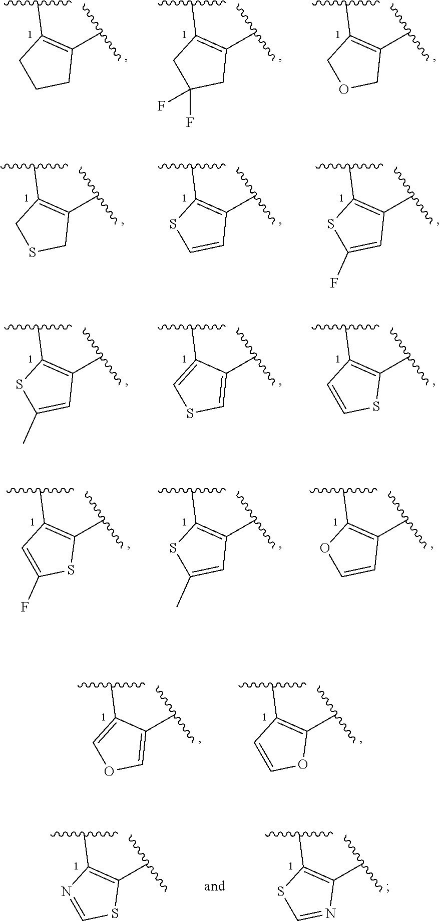
R² is H;
B is selected from 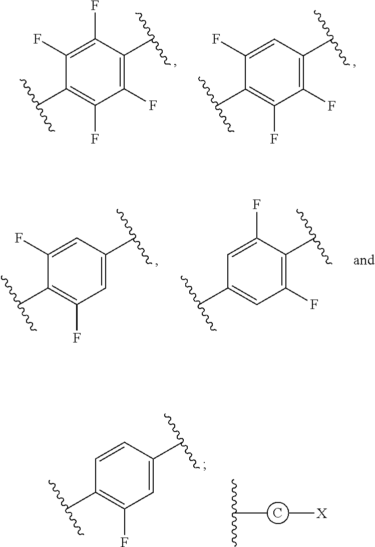
is selected from

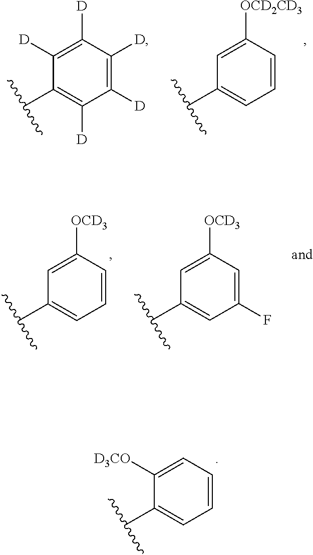

9. A compound of Formula (I) according to claim 1, which is selected from 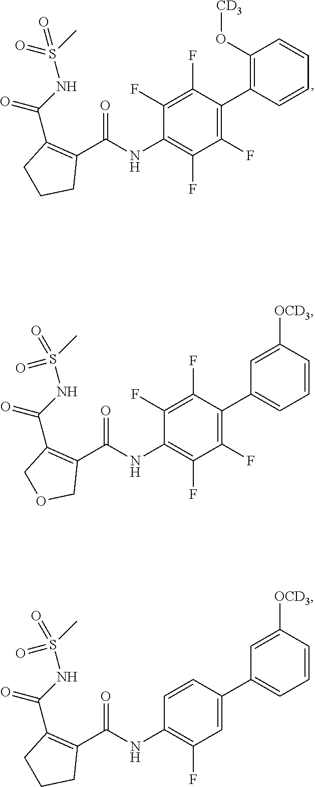 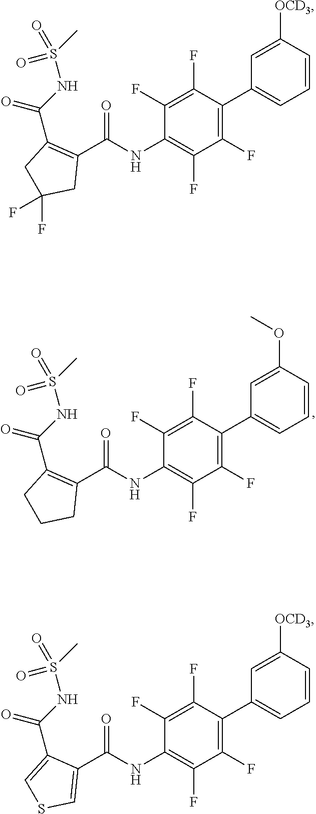 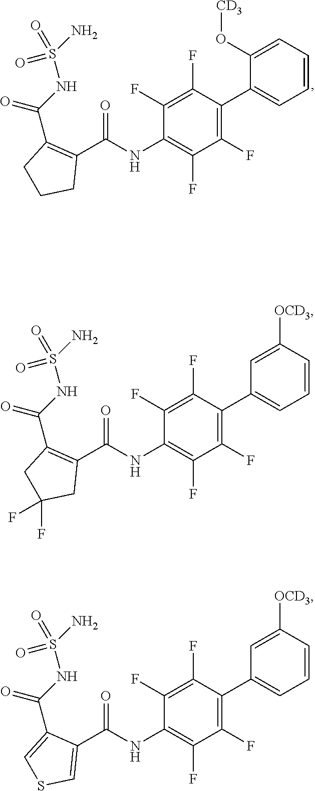 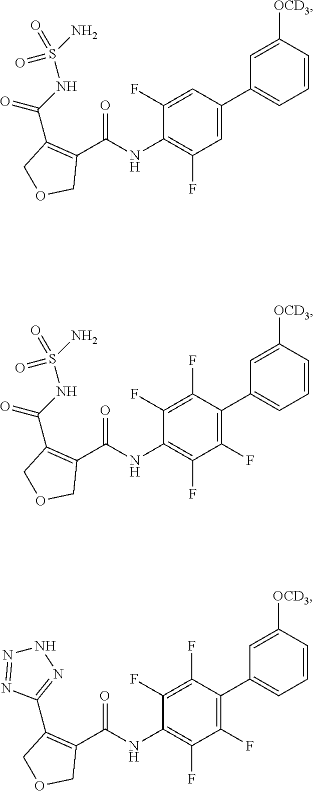 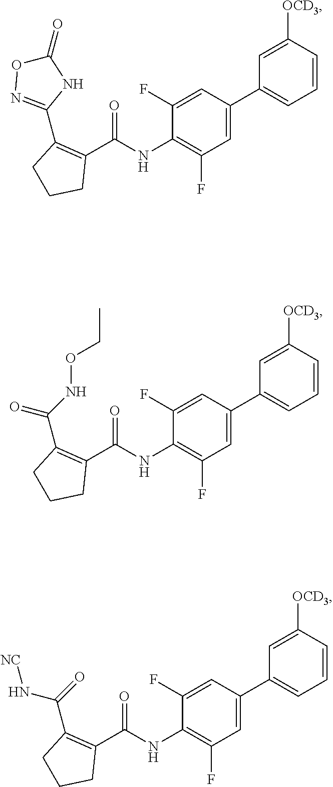 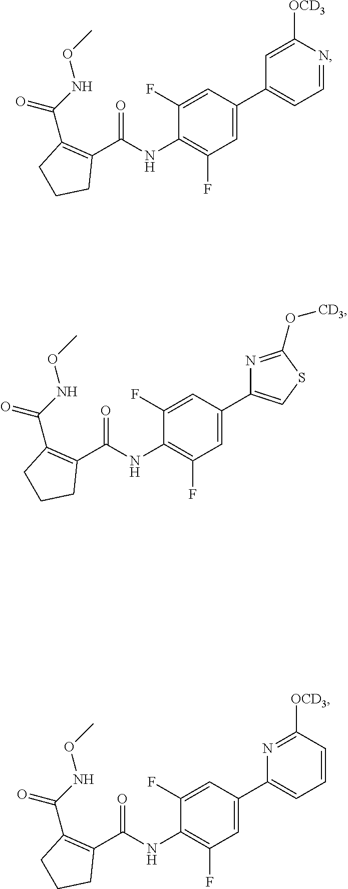 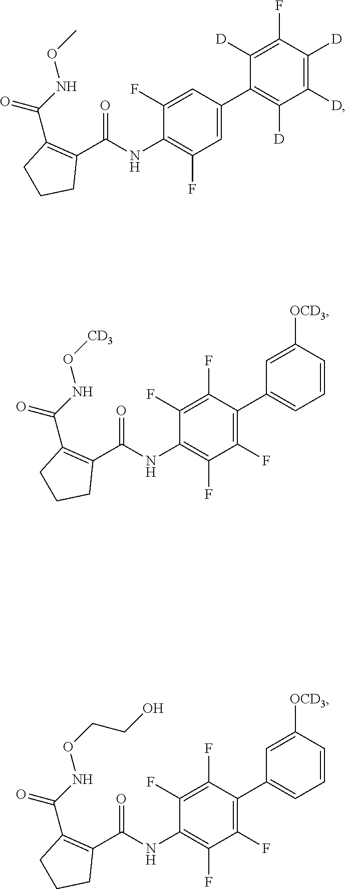 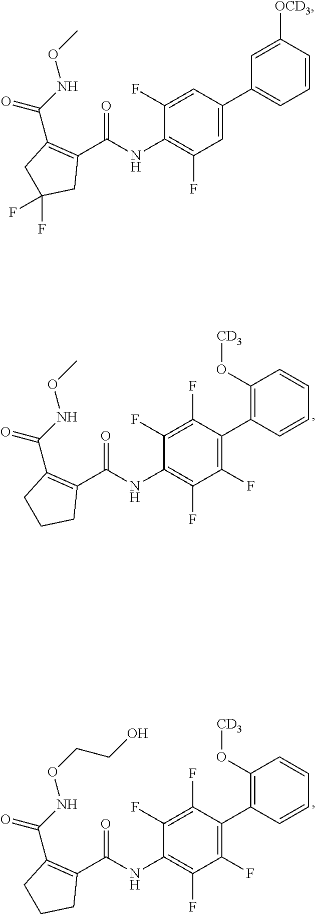 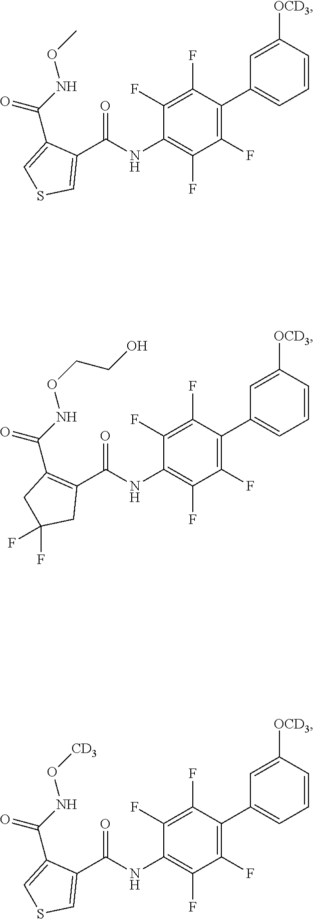 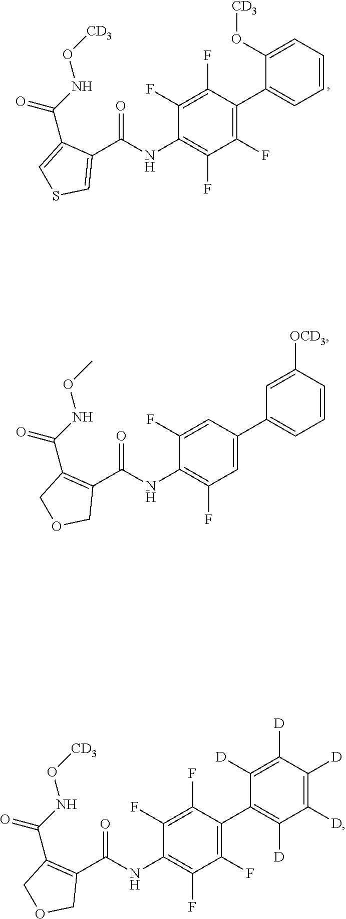 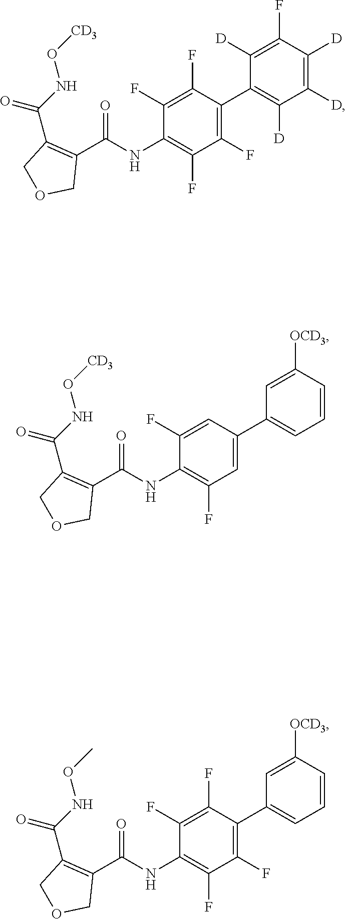 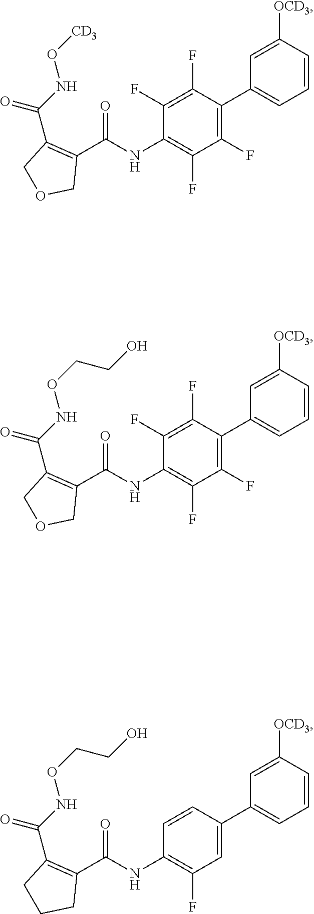 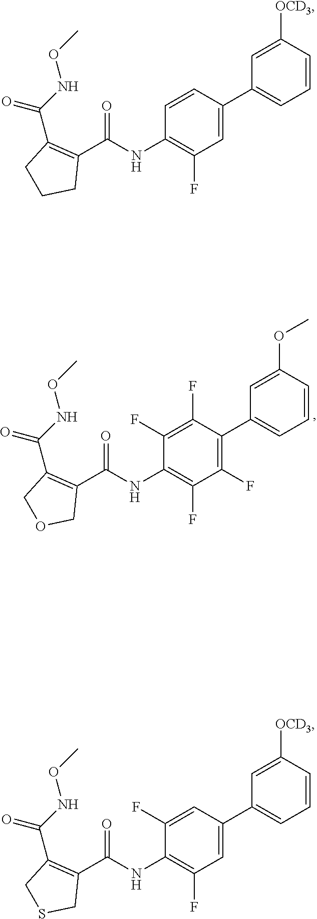 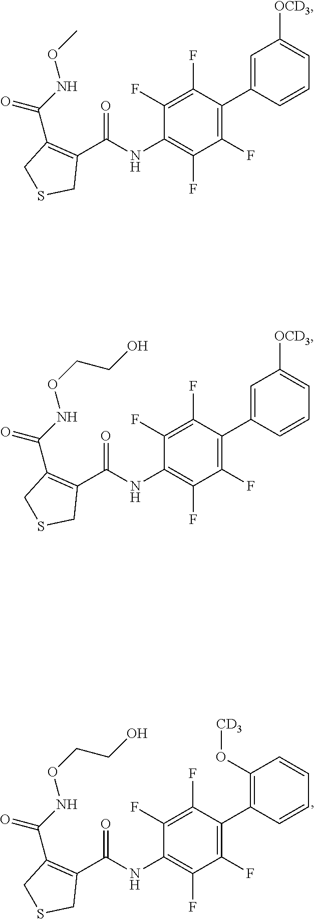 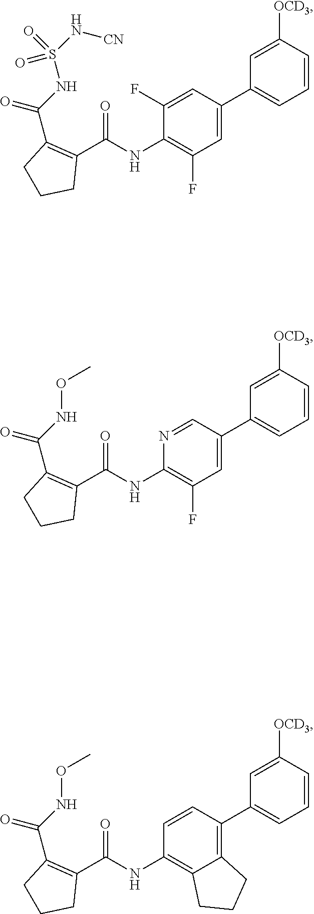 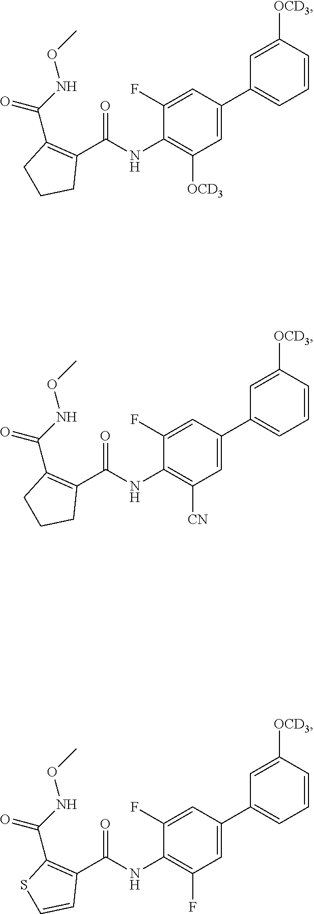 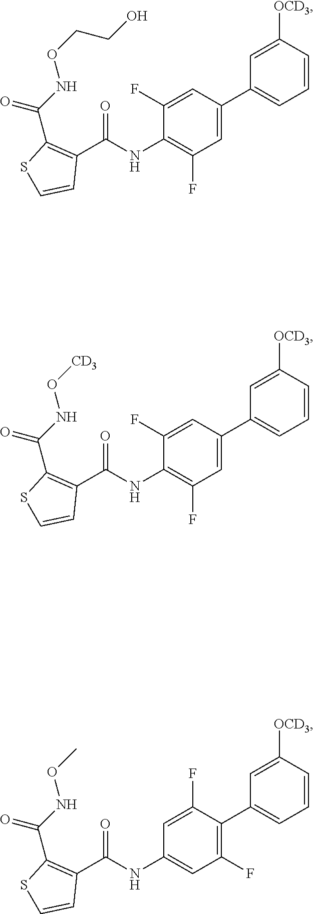 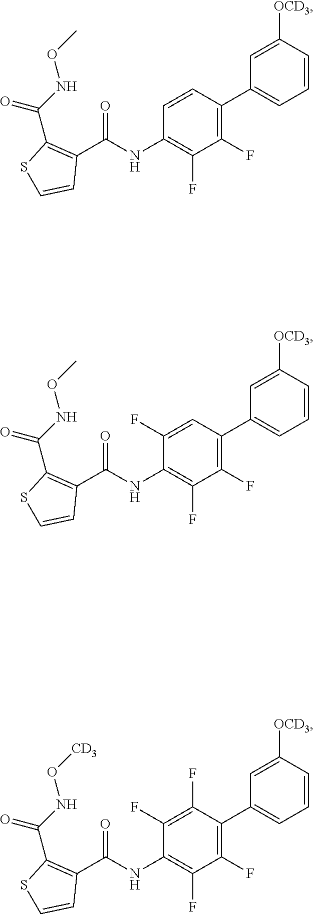 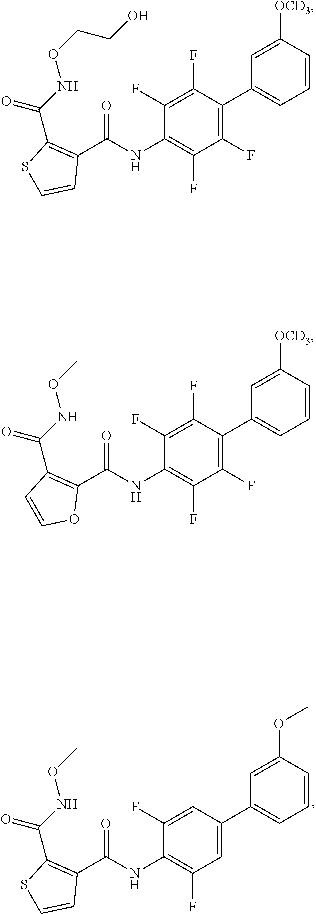 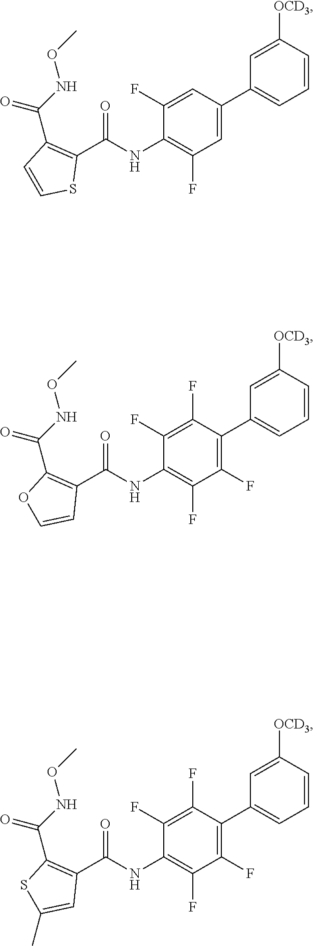 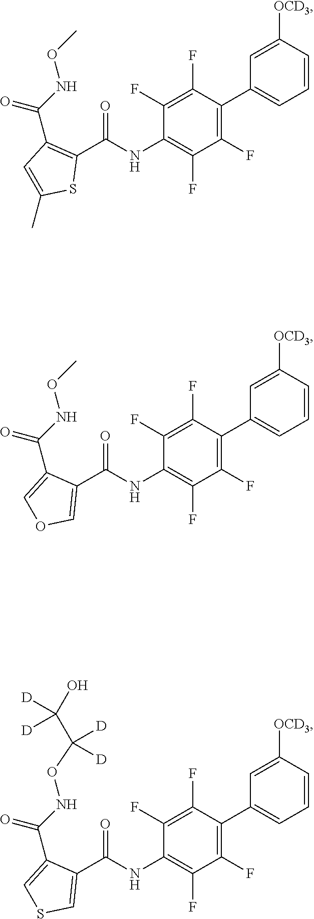 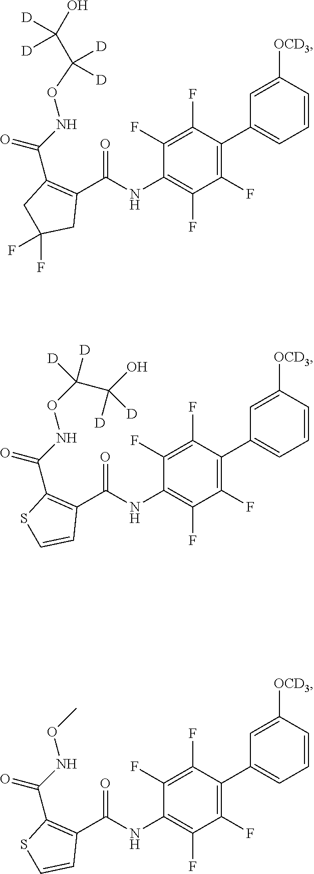 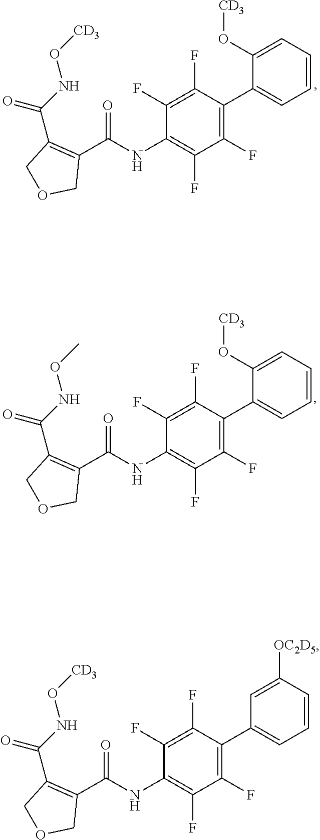 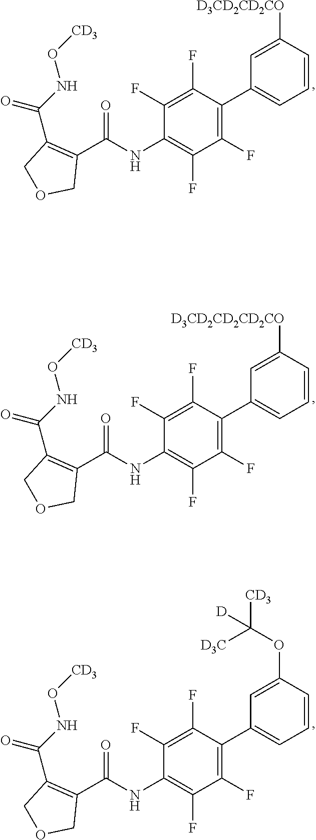 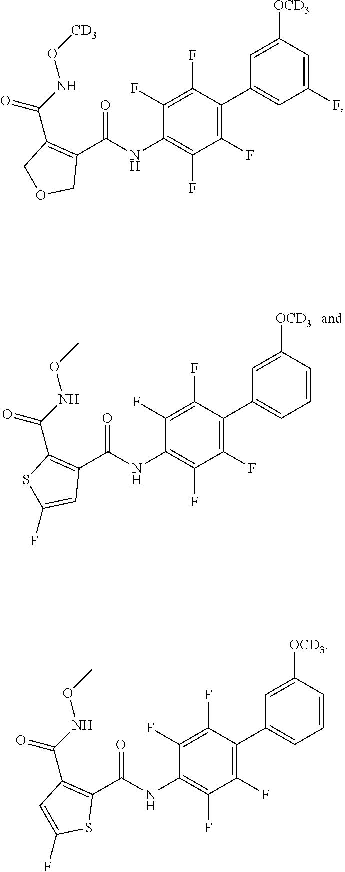
or a solvate or pharmaceutically acceptable salt thereof.

10. A method of treating a condition in a subject in need thereof, comprising administering to the subject a compound according to claim 1.

11. The method of claim 10, wherein the condition is a disease, disorder, therapeutic indication or medical condition amenable to treatment with DHODH inhibitors.

12. The method of claim 10, wherein the condition is selected from rheumatism, an acute immunological disorder, an autoimmune disease, a disease caused by malignant cell proliferation, an inflammatory disease, a disease that are caused by protozoal infestations in humans and animals, a disease caused by a viral infection, Pneumocystis carinii, fibrosis, uveitis, rhinitis, asthma, transplantation, and arthropathy.

13. The method of claim 10, wherein the condition is selected from graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

14. A pharmaceutical composition comprising a compound according to claim 1 and a pharmaceutically acceptable carrier or excipient.

15. A pharmaceutical composition of claim 14, further comprising one or more additional therapeutic agents selected from anti-inflammatory agents, anti-viral agents, immunosuppressive and/or immunomodulatory agents, steroids, non-steroidal anti-inflammatory agents, antihistamines, analgesics and suitable mixtures thereof.
